(19) United States
(12) Patent Application Publication
(10) Pub. No.: US 2004/0224366 A1
(43) Pub. Date: Nov. 11, 2004

(54) Valency platform molecules comprising aminooxy groups

(76) Inventors: David S. Jones, San Diego, CA (US); Huong-Thu Ton-Nu, San Diego, CA (US); Anping Tao, San Diego, CA (US); Tong Xu, San Diego, CA (US); Jeffrey Robert Hammaker, San Diego, CA (US)

Correspondence Address: MORRISON & FOERSTER LLP, 755 PAGE MILL RD, PALO ALTO, CA, 94304-1018, US

(21) Appl. No.: 10/867,874

(22) Filed: Jun. 14, 2004

Related U.S. Application Data
(63) Continuation of application No. 09/590,592, filed on Jun. 8, 2000.
(60) Provisional application No. 60/138,260, filed on Jun. 8, 1999.

(51) Int. Cl. G01N 33/53; C07C 291/00; C07F 7/04

(52) U.S. Cl. 435/7.1; 556/413; 564/297

(57) ABSTRACT

Molecules comprising aminooxy groups are provided, wherein the aminooxy groups provide attachment sites for the covalent attachment of other molecules. In one embodiment, polyoxyethylene molecules comprising aminooxy groups are provided that can be conjugated to wide variety of biologically active molecules including poly(amino acids). In another embodiment, valency platform molecules comprising aminooxy groups are provided. The aminooxy groups can be used to form covalent bonds with biological molecules such as poly(amino acids). The aminooxy groups can, for example, react with poly(amino acids) modified to contain carbonyl groups, such as glyoxyl groups, to form a conjugate of the valency platform molecule and the biologically active molecule via an oxime bond. The valency platform molecules comprising aminooxy groups are advantageously reactive in the formation of conjugates, and they also can be readily synthesized to form a composition with very low polydispersity.

### CROSS REFERENCE TO RELATED APPLICATIONS

This application is a continuation of U.S. application Ser. No. 09/590,592, filed Jun. 8, 2000 which claims the benefit of U.S. Provisional Application No. 60/138,260, filed Jun. 8, 1999, the disclosure of both which are incorporated herein by reference in their entirety.

### TECHNICAL FIELD

This application relates to molecules comprising aminooxy groups that can be covalently attached to other molecules. In particular, this application relates to valency platform molecules comprising aminooxy groups to which one or more molecules, such as biologically active molecules, may be attached to form a conjugate.

### BACKGROUND ART

A “valency platform” is a molecule with one or more (and typically multiple) attachment sites which can be used to covalently attach biologically active molecules of interest to a common scaffold. The attachment of biologically active molecules to a common scaffold provides multivalent conjugates in which multiple copies of the biologically active molecule are covalently linked to the same platform. A “defined” or “chemically defined” valency platform is a platform with defined structure, thus a defined number of attachment points and a defined valency. A defined valency platform conjugate is a conjugate with defined structure and has a defined number of attached biologically active compounds. Examples of biologically active molecules include oligonucleotides, peptides, polypeptides, proteins, antibodies, saccharides, polysaccharides, epitopes, mimotopes, drugs, and the like. For example, the biologically active compounds may interact specifically with proteinaceous receptors.

Certain classes of chemically defined valency platforms, methods for their preparation, conjugates comprising them, and methods for the preparation of such conjugates, have been described in the U.S. Pat. Nos. 5,162,515; 5,391,785; 5,276,013; 5,786,512; 5,726,329; 5,268,454; 5,552,391; 5,606,047; and 5,663,395. Valency platform molecules comprising carbamate linkages are described in U.S. Provisional Patent Application Ser. No. 60/111,641; and U.S. Ser. No. 09/457,607, filed Dec. 8, 1999; now U.S. Pat. No. 6,458,953, issued Oct. 1, 2002.

### DISCLOSURE OF THE INVENTION

Molecules comprising aminooxy groups are provided, as well as conjugates thereof with other molecules such as biologically active molecules, and methods for their synthesis. The aminooxy groups provide attachment sites for the covalent attachment of other molecules.

In one embodiment, polyethylene oxide molecules, or more particularly, polyethylene glycol molecules, comprising aminooxy groups are provided that can be conjugated to a wide variety of biologically active molecules including poly(amino acids). In another embodiment, valency platform molecules comprising aminooxy groups are provided. The aminooxy groups can be used to form covalent bonds with biological molecules, such as poly(amino acids). The aminooxy groups can, for example, react with poly(amino acids) modified to contain carbonyl groups, such as glyoxyl groups, to form a conjugate of the valency platform molecule and the biologically active molecule via an oxime bond. The valency platform molecules comprising aminooxy groups are advantageously reactive in the formation of conjugates, and they also can be readily synthesized to form a composition with very low polydispersity.

Molecules comprising aminooxy groups, preferably 3 or more aminooxy groups, such as valency platform molecules comprising aminooxy groups, can be covalently linked to one or more, or, for example, 3 or more, biologically active molecules, including, for example, oligonucleotides, peptides, polypeptides, proteins, antibodies, saccharides, polysaccharides, epitopes, mimotopes, or drugs.

In one embodiment, a molecule comprising aminooxy groups is provided, wherein the molecule comprises oxyalkylene groups, e.g., oxyethylene groups or polyoxyethylene groups. The molecule may comprise, e.g., at least 3 aminooxy groups, or 4, 5 or more aminooxy groups.

As used herein “oxyethylene, oxypropylene and oxyalkylene” are used interchangably with “ethylene oxide, propylene oxide and alkylene oxide”.

In another embodiment, there is provided a valency platform molecule comprising aminooxy groups. In one preferred embodiment, the valency platform molecule comprises at least 3 aminooxy groups. The valency platform molecule may further comprise oxyalkylene groups, e.g., oxyethylene or polyoxyethylene groups, e.g., —(CH₂CH₂O)ₙ—, wherein n is 200 to 500.

Also provided is a composition comprising a molecule, such as a valency platform molecule, such as those disclosed herein, comprising aminooxy groups and having a polydispersity less than 1.2, e.g., less than 1.1, or less than 1.07.

In one embodiment, there is provided a valency platform molecule having the formula:

R—(ONH₂)ₘ Formula 1

wherein in one embodiment:

m is 1-50 or more, e.g., 3-50; and

R is an organic moiety comprising 1-1000, or 10,000 atoms or more selected from the group consisting of H, C, N, O, P, Si and S atoms.

In another embodiment, there is provided a valency platform molecule having the formula:

R^{c}[G₁(ONH₂)ₙ]_{y}; Formula 2

wherein in one embodiment:

y is 1 to 16;

n is 1 to 32;

wherein in one embodiment the product of y*n (y multiplied by n) is at least 3; and

R^{c} and each G₁ are independently an organic moiety.

In one embodiment, R^{c} and each G₁ are independently an organic moiety comprising atoms selected from the group of H, C, N, O, P, Si and S atoms, and optionally comprise oxyalkylene groups. The molecules may be provided in a composition having a polydispersity less than 1.2.

In another embodiment, a valency platform molecule is provided having a formula selected from the group consisting of:

R^{c}[O—C(═O)—NR¹-G₂-(ONH₂)ₙ]_{y} Formula 3;

R^{c}[C(═O)—NR¹-G₂-(ONH₂)ₙ]_{y} Formula 4;

R^{c}[NR¹—C(═O)-G₂-(ONH₂)ₙ]_{y} Formula 5;

R^{c}[NR¹—C(═O)—O-G₂-(ONH₂)ₙ]_{y} Formula 6;

R^{c}[R¹C═N—O-G₂-(ONH₂)ₙ]_{y} Formula 7; and

R^{c}[S-G₂(ONH₂)ₙ]_{y} Formula 8;

wherein, for example:

y is 1 to 16;

n is 1 to 32;

wherein in one embodiment the product of y*n (y multiplied by n) is at least 3;

R¹ is H, alkyl, heteroalkyl, aryl, heteroaryl or G₂-(ONH₂)ₙ; and

R^{c} and each G₂ are independently organic moieties comprising atoms selected from the group of H, C, N, O, P, Si and S atoms.

In one embodiment, R^{c} and each G₂ independently are selected from the group consisting of:

hydrocarbyl groups consisting only of H and C atoms and having 1 to 200 carbon atoms;

organic groups consisting only of carbon, oxygen, and hydrogen atoms, and having 1 to 200 carbon atoms;

organic groups consisting only of carbon, oxygen, nitrogen, and hydrogen atoms, and having from 1 to 200 carbon atoms;

organic groups consisting only of carbon, oxygen, sulfur, and hydrogen atoms, and having from 1 to 200 carbon atoms;

organic groups consisting only of carbon, oxygen, sulfur, nitrogen and hydrogen atoms and having from 1 to 200 carbon atoms.

In one embodiment of the valency platform molecule, R^{c} is selected from the group consisting of a C1-200 hydrocarbon moiety; a C1-200 alkoxy moiety; and a C1-200 hydrocarbon moiety comprising an aromatic group.

R^{c} optionally may comprise an oxyalkylene moiety, such as an oxyethylene moiety (—CH₂CH₂O—). In one embodiment R^{c} comprises oxyethylene units:

—(CH₂CH₂O)ₙ—;

wherein n is 1-500, e.g., 200-500, 1-200, 1-100 or 1-20.

In one embodiment, each G₂ independently comprises a functional group selected from the group consisting of alkyl, heteroalkyl, aryl, and heteroaryl.

In another embodiment, each G₂ independently comprises a functional group selected from the group consisting of a C1-200 hydrocarbon moiety; a C1-200 alkoxy moiety; and a C1-200 hydrocarbon moiety comprising an aromatic group.

Each G₂ independently can comprise an oxyalkylene moiety, such as an oxyethylene moiety (—CH₂CH₂O—). In one embodiment, each G₂ independently comprises oxyethylene units:

—(CH₂CH₂O)ₙ—;

wherein n is 1-500, e.g., 1-200, 200-500, 1-100 or 1-20.

In one embodiment of the valency platform molecule each G₂ independently comprises a functional group selected from the group consisting of amine; amide; ester; ether; ketone; aldehyde; carbamate; thioether; piperazinyl; piperidinyl; alcohol; polyamine; polyether; hydrazide; hydrazine; carboxylic acid; anhydride; halo; sulfonyl; sulfonate; sulfone; cyanate; isocyanate; isothiocyanate; formate; carbodiimide; thiol; oxime; imine; aminooxy; and maleimide.

In one embodiment, in the valency platform molecules, each G₂—ONH₂ is independently selected from the moieties shown in FIG. 17.

In another embodiment, valency platform molecules are synthesized using a linker comprising an aminooxy or protected aminooxy group on one end. The other end may include an an amine, as illustrated in compounds 11 and 100 in Examples 3 and 17, and in FIGS. 3 and 25; an acid carbonate ester as illustrated by compounds 18 and 28, and Examples 4 and 6, as well as FIGS. 4 and 7; a thiol, as illustrated by compounds 22a and 22b, Examples 5a and 5b, and FIGS. 5 and 6; an aminooxy, as illustrated by compound 37, Example 8 and FIG. 9), or a carboxylic acid or activated derivative as illustrated by compound 105 and 106, Examples 16 and 20, and FIGS. 24 and 28.

In another embodiment, compounds of Formulas 9-13 shown in FIG. 19 are provided. In Formulas 9-13, in one embodiment, R^{c} and G₂ are as defined above, and n is about 1-500, e.g., 200-500, 1-200, 1-100 or 1-50.

In a further embodiment, valency platform molecules are provided having the structure:

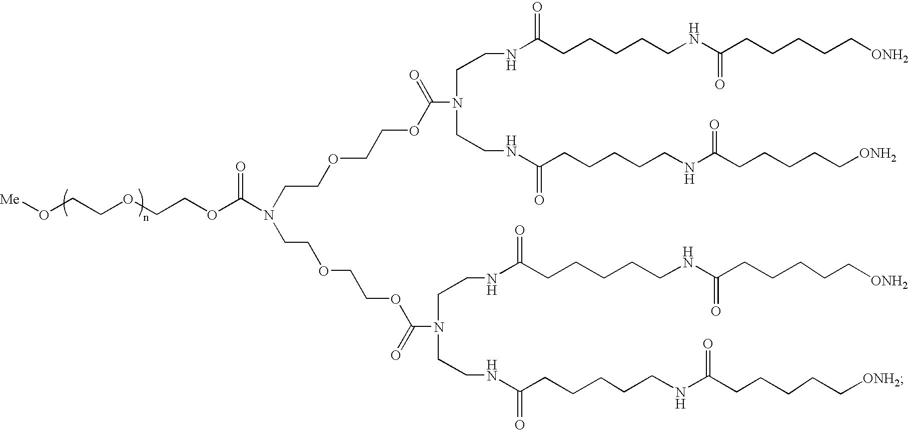

where n is about 503 or e.g., more than about 500, more than about 600, or more than about 700 or 800 or more;

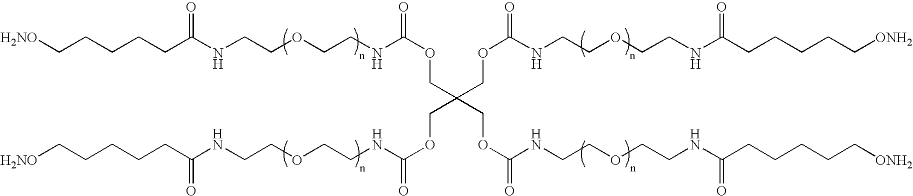

where n is about 112, or e.g., more than about 500, more than about 600, or more than about 700 or 800 or more;

or the structure:

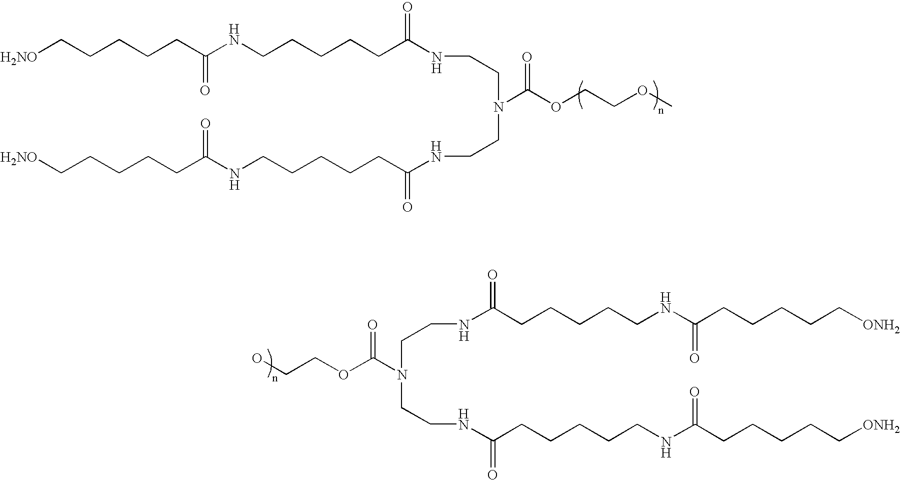

where n is about 481, or e.g., more than about 500, more than about 600, or more than about 700 or 800 or more.

Also provided are conjugates of a molecule comprising aminooxy groups, such as any of the valency platform molecules disclosed herein, and a biologically active molecule. The biologically active molecule may include, for example, poly(saccharides), poly(aminoacids), nucleic acids, lipids and drugs, and combinations thereof. The conjugates include an oxime conjugate or modified form thereof, such as reduction products, such as aminooxy, and alkylated forms.

Also provided is a method of making a conjugate of a molecule comprising aminooxy groups, such as any of the valency platform molecules disclosed herein, and a biologically active molecule, wherein the method comprises reacting aminooxy groups on the molecule comprising aminooxy groups, such as a valency platform molecule, with a reactive functional group, such as the carbonyl, for example, of an aldehyde or ketone group, on the biologically active molecule to form an oxime conjugate. In the embodiment wherein the biologically active molecule is a poly(amino acid), the method may further comprise modifying the poly(amino acid) to include a terminal aldehyde group prior to the conjugation.

Also provided are pharmaceutically acceptable compositions comprising the conjugates disclosed herein, optionally in a pharmaceutically acceptable carrier.

### BRIEF DESCRIPTION OF THE DRAWINGS

FIG. 1 is a scheme showing the synthesis of a transaminated polypeptide.

FIG. 2 is a scheme showing the synthesis of an aminooxyacetyl valency platform molecule.

FIG. 3 is a scheme showing the synthesis of an alkylaminooxy valency platform molecule.

FIG. 4 is a scheme showing another embodiment of a synthesis of an alkylaminooxy valency platform molecule.

FIGS. 5 and 6 are schemes showing the synthesis of an alkylaminooxy valency platform molecule comprising thioether functionalities.

FIG. 7 is a scheme showing another embodiment of the synthesis of an alkylaminooxy valency platform molecule.

FIG. 8 is a scheme showing another embodiment of the synthesis of an alkylaminooxy valency platform molecule.

FIG. 9 is a scheme showing the synthesis of an alkylaminooxy valency platform molecule comprising piperazine moieties and oxime linkages.

FIG. 10 is a scheme showing synthesis of an alkylaminooxy valency platform molecule.

FIG. 11 is a scheme showing the synthesis of a conjugate of an aminooxyacetyl valency platform molecule comprising piperazine moieties and a polypeptide.

FIG. 12 is a scheme showing the synthesis of the conjugate of an alkylaminooxy valency platform molecule and a polypeptide.

FIG. 13 is a graph comparing the rate of conjugate formation for a model alkylaminooxy compound and a model aminooxyacetyl compound.

FIG. 14 is a scheme showing the synthesis of a model alkylaminooxy compound and a model aminooxyacetyl compound and their reaction with a glyoxylated polypeptide.

FIG. 15 is a scheme showing another embodiment of the synthesis of the conjugate of an alkylaminooxy valency platform molecule and a poly(amino acid).

FIG. 16 is a scheme showing an alternate method of preparing a polypeptide using a thiol containing aminooxy linker and a haloacetyl platform.

FIG. 17 shows exemplary G₂—ONH₂ groups on a valency platform molecule.

FIG. 18 shows some exemplary Formulas for valency platform molecules comprising aminooxy groups.

FIG. 19 shows another embodiment of Formulas for valency platform molecules comprising aminooxy groups.

FIG. 20 shows embodiments of valency platform molecules comprising aminooxy groups.

FIG. 21 shows embodiments of further valency platform molecules comprising aminooxy groups.

FIG. 22 shows additional embodiments of valency platform molecules comprising aminooxy groups.

FIG. 23 shows a scheme for the synthesis of compound 85.

FIG. 24 shows a scheme for the synthesis of compound 86.

FIG. 25 shows a scheme for the synthesis of compound 91.

FIG. 26 shows a scheme for the synthesis of compound 92.

FIG. 27 shows a scheme for the synthesis of compound 113.

FIG. 28 shows a scheme for the synthesis of multivalent platform molecules comprising polyethylene oxide groups of varying molecular weight.

FIG. 29 shows a scheme for the synthesis of multivalent platform molecules comprising polyethylene oxide groups and branching groups.

FIG. 30 shows a scheme for the synthesis of a multivalent platform molecule comprising a polyethylene oxide group and a branching group.

FIG. 31 shows a scheme for the synthesis of multivalent platform molecules comprising polyethylene glycol groups.

FIG. 32 shows a scheme for the synthesis of a multivalent molecule comprising a polyethylene glycol group.

FIG. 33 shows the structure of some exemplary conjugates of valency platform molecules and biologically active molecules.

FIG. 34 shows the synthesis of an an octameric platform comprising polyethylene oxide, wherein n is, for example, 112.

FIG. 35 shows the synthesis of a valency platform molecule comprising two polyethylene oxide groups, wherein n is, for example, 500 or more.

### MODES FOR CARRYING OUT THE INVENTION

Molecules comprising aminooxy groups are provided. The aminooxy groups may be provided on molecules such as polymers, for example at the terminal position, to provide attachment sites for the covalent attachment of other molecules, such as biologically active molecules. For example, a wide variety of polymers, such as poly(alkyleneoxide) polymers, including poly(ethyleneoxide) polymers, in particular polyethylene glycols, can be modified to contain aminooxy groups. The aminooxy groups are advantageous because they can be used to react rapidly and in good yields with other molecules containing reactive groups, preferably aldehyde or ketone groups, to form a covalent conjugate with the other molecule. Aminooxy groups provide improved results in reacting with an aldehyde or ketone to form a stable conjugate in the form of a C═N bond, in comparison with other nitrogen containing functional groups, such as amines, hydrazides, carbazides and semicarbazides. The aminooxy groups permit both reduced reaction time and increased yield of product.

Other molecules that can be modified to include aminooxy groups include branched, linear, block, and star polymers and copolymers, for example those comprising polyoxyalkylene moieties, such as polyoxyethylene molecules, and in particular polyethylene glycols. The polyethylene glycols preferably have a molecular weight less than about 10,000 daltons. In one embodiment, polymers with low polydispersity may be used. For example, polyoxypropylene and polyoxyethylene polymers and copolymers, including polyethylene glycols may be modified to include aminooxy groups, wherein the polymers have a low polydispersity, for example, less than 1.5, or less than 1.2 or optionally less than 1.1 or 1.07. Preferably, the polymers comprise at least 3 aminooxy groups, or at least 4, 5, 6, 7, 8, or more.

Nonpolymeric molecules also can be modified to include aminooxy groups as disclosed herein. For example, chemically defined non-polymeric valency platform molecules, such as those described in U.S. Pat. No. 5,552,391 can be modified to include aminooxy groups.

Also provided are compositions comprising such molecules and conjugates, for example in a pharmaceutically acceptable form, for example, in a pharmaceutically acceptable carrier. Carriers for different routes of administration, including oral, intravenous, and aerosol administration are described in the art, for example, in “Remington: The Science and Practice of Pharmacy,” Mack Publishing Company, Pennsylvania, 1995, the disclosure of which is incorporated herein by reference. Carriers can include, for example, water, saccharides, polysaccharides, buffers, excipients, and biodegradable polymers such as polyesters, polyanhydrides, polyamino acids and liposomes.

Pharmaceutically acceptable compositions are compositions in a form suitable for administration to an individual, for example, systemic or localized administration to individuals in unit dosage forms, sterile parenteral solutions or suspensions, sterile non-parenteral solutions or oral solutions or suspensions, oil in water or water in oil emulsions and the like.

Valency Platforms

In one aspect, valency platform molecules comprising aminooxy groups, conjugates thereof with molecules such as biologically active molecules, and methods for the preparation of such platforms and conjugates are provided.

A variety of valency platform molecules are known in the art. Preferred are chemically defined valency platform molecules. Methods for making valency platform molecules are described, for example, in U.S. Pat. Nos. 5,162,515; 5,391,785; 5,276,013; 5,786,512; 5,726,329; 5,268,454; 5,552,391; 5,606,047; 5,663,395 and 5,874,409, as well as in U.S. Serial No. 60/111,641 and PCT Application No. PCT/US97/10075; published as PCT Publication No. WO 97/46251, Dec. 11, 1997. In general, these platforms contain core groups or branched core groups which terminate in hydroxyl groups, carboxyl groups, amino groups, aldehydes, ketones, or alkyl halides. These groups can be further modified to give the desired reactive groups, and to obtain a valency platform molecule comprising preferably at least three aminooxy groups.

Valency platforms are prepared from core groups which contain the desired valence. A chain can provide a valence of one or two, depending on how the chain is terminated. Chains which are branched can provide a valence of three or more depending on the number of branches or side chains. For example, triethylene glycol, has a valence of two, ethanol has a valence of one, pentaerythritol has a valence of four. These are chains which terminate in hydroxyl groups which can be further modified to provide desired reactive groups. Chains can also terminate in other groups such as amines, thiols, alkyl halides, carboxyl groups, aldehydes, ketones, or other groups which can be further modified.

These chains can serve as core groups. The valence of a core, group can be increased by derivatizing the terminal functionality with branching moieties. For instance, triethylene glycol, with a valence of two, can be converted to a platform with a valence of four by converting triethylene glycol to a bis-chloroformate derivative. Reaction of the bis-chloroformate with an appropriately substituted diethylenetriamine derivative provides a tetravalent platform, as illustrated in Example 6. Similarly, reaction of triethyleneglycol bis-chloroformate with iminodiacetic acid can provide a tetravalent platform terminated in carboxyl groups, as shown in Example 7.

Methods known in the art for making valency platform molecules, include, for example, a propagation method, or segmented approach. Such methods can be modified, using the appropriate reagents, to provide aminooxy groups on the resulting molecule. For example, reactive groups, such as halide groups, hydroxy groups, amino groups, aldehydes, ketones, or carboxyl groups, may be reacted to attach molecules, such as linkers, that comprise aminooxy groups that are optionally protected. Exemplary methods are demonstrated in the Examples herein.

The advantages of the use of valency platform molecules include the ease of synthesis, the ability to adjust the length and water solubility of the “arms” of the valency platform by using, for example, different alkyleneoxy or dialcoholamine groups, and the ability to further attenuate the properties of the valency platform by choice of the core group.

In one aspect, valency platform molecules are provided that are substantially monodisperse. The aminooxy valency platform molecules advantageously have a narrow molecular weight distribution. A measure of the breadth of distribution of molecular weight of a sample of an aminooxy valency platform molecule is the polydispersity of the sample. Polydispersity is used as a measure of the molecular weight homogeneity or nonhomogeneity of a polymer sample. Polydispersity is calculated by dividing the weight average molecular weight (Mw) by the number average molecular weight (Mn). The value of Mw/Mn is unity for a perfectly monodisperse polymer. Polydispersity (Mw/Mn) is measured by methods available in the art, such as gel permeation chromatography. The polydispersity (Mw/Mn) of a sample of an aminooxy valency platform molecule is preferably less than 2, more preferably, less than 1.5, or less than 1.2, less than 1.07, less than 1.02, or, e.g., about 1.05 to 1.5 or about 1.05 to 1.2. Typical polymers generally have a polydispersity of 2-5, or in some cases, 20 or more. Advantages of the low polydispersity property of the valency platform molecules include improved biocompatibility and bioavailability since the molecules are substantially homogeneous in size, and variations in biological activity due to wide variations in molecular weight are minimized. The low polydispersity molecules thus are pharmaceutically optimally formulated and easy to analyze. Further there is controlled valency of the population of molecules in the sample.

In some embodiments, the valency platform molecule may be described as “dendritic,” owing to the presence of successive branch points. Dendritic valency platform molecules possess multiple termini, typically 4 or more termini, e.g., 8 termini, or 16 termini.

Note that the Formulas disclosed herein are intended to encompass both “symmetric” and “non-symmetric” valency platforms. In one embodiment, the valency platform is symmetric. In another embodiment, the valency platform is non-symmetric.

General Formulas

In one embodiment, provided are valency platform molecules comprising terminal aminooxy groups, for example, 1 to 100, e.g, 1-50, 2-16, 4-16, or e.g., 2, 3, 4, 8, 16, 32 or more aminooxy groups. In one embodiment, a valency platform molecule is provided that has at least 3 or 4 aminooxy groups, and optionally further comprises oxyalkylene groups, such as oxyethylene groups or polymers thereof. In one embodiment, a valency platform molecule is provided, having the formula:

R—(ONH₂)ₘ Formula 1

wherein:

m is 1 to 100, for example, 1-50, 1-16, 2-16, 4-16, or, e.g., 2, 4, 8, 16, 32 or more, and in one embodiment is at least 3, e.g., 3-50; and

R is an organic moiety, for example, comprising atoms, e.g., 1 to 10,000 atoms, 1 to 1000 atoms, or e.g., 1-100 atoms, including, for example, H, C, N, O, P, Si and S atoms, as well as halogen atoms. For example, R may include between 1 to 1000 or, e.g., 1-100, C, H, N, and O atoms.

In another embodiment, the valency platform molecule has the formula:

R^{c}[G₁(ONH₂)ₙ]_{y}; Formula 2

wherein:

y is, for example, 1 to 100, e.g, 1-50, 1-32, 1-16, 2-16, 4-16, or e.g., 1, 2, 3, 4, 8, 16, 32 or more;

n is, for example, 1 to 100, e.g, 1-50, 1-32, 1-16, 2-16, 4-16, or e.g., 2, 3, 4, 8, 16, 32 or more;

wherein, in one embodiment, the product of y*n (y multiplied by n) is at least 3; and

R^{c} and each G₁ are independently organic moieties, for example, comprising atoms selected from the group of H, C, N, O, P, Si and S atoms, for example, less than 1000 atoms, 1,000 to 10,000 or more.

In one embodiment R^{c} is as defined below, and G₁ is as G₂ is defined below. In one embodiment, the molecule of Formula 2 comprises oxyalkylene groups.

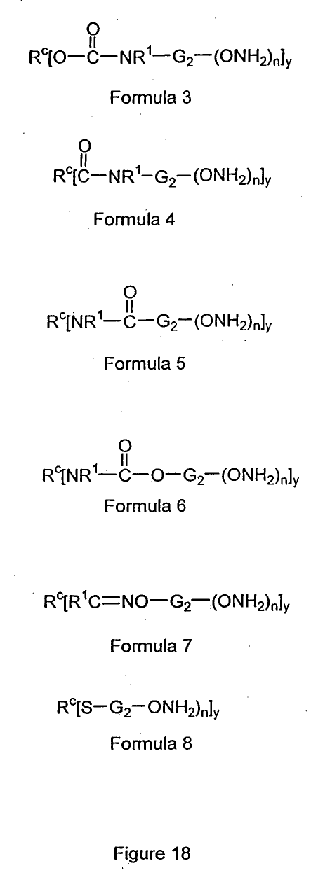

In another embodiment, a valency platform molecule is provided, having one of the following formulas also shown in FIG. 18:

R^{c}[O—C(═O)—NR¹-G₂-(ONH₂)ₙ]_{y} Formula 3;

R^{c}[C(═O)—NR¹-G₂-(ONH₂)ₙ]_{y} Formula 4;

R^{c}[NR¹—C(═O)-G₂-(ONH₂)ₙ]_{y} Formula 5;

R^{c}[NR¹—C(═O)—O-G₂-(ONH₂)ₙ]_{y} Formula 6;

R^{c}[R¹C═N—O-G₂-(ONH₂)ₙ]_{y} Formula 7; or

R^{c}[S-G₂(ONH₂)ₙ]_{y} Formula 8;

wherein, in one embodiment:

y is 1 to 100, e.g, 1-50,1-32,1-16, 2-16, 4-16, or e.g., 1, 2, 3, 4, 6, 8, 16, 32, 64 or more;

n is 1 to 100, e.g, 1-50, 1-32, 1-16, 2-16, 4-16, or e.g., 2, 3, 4, 6, 8, 16, 32, 64 or more;

wherein in one embodiment the product of y*n (y multiplied by n) is at least 3;

R¹ if present is, for example, H, alkyl, heteroalkyl, aryl, heteroaryl, or optionally is -G₂(ONH₂)ₙ as defined herein; and

R^{c} and each G₂ are independently organic moieties, for example, comprising atoms selected from the group of H, C, N, O, P, Si and S atoms, or optionally halogen atoms, for example, 1 to 10,000, 1 to 1000 atoms, or 1 to 100 atoms.

R¹ thus can be, in one embodiment, any alkyl moiety including carbon and hydrogen groups, such as methyl, ethyl or propyl, or other hydrocarbon including straight chain, branched or cyclic structures, which may be saturated or unsaturated, or may be a heteroalkyl group further comprising, for example O, S or N atoms, or may be an aryl or heteroaryl group.

In one embodiment, R^{C} and each G₂ independently comprise, e.g., a straight chain, branched or cyclic structure, and are independently selected from the group consisting of:

hydrocarbyl groups, consisting of only H and C atoms and having 1 to 5,000,1-500, 1 to 200, 1 to 100, or, e.g., 1 to 20 carbon atoms;

organic groups consisting only of carbon, oxygen, and hydrogen atoms, and having 1-5,000, 1 to 500, 1 to 200, 1 to 100, or, e.g., 1 to 20 carbon atoms;

organic groups consisting only of carbon, oxygen, nitrogen, and hydrogen atoms, and having from 1-5,000, 1 to 500, 1 to 200, 1 to 100, or, e.g., 1 to 20 carbon atoms;

organic groups consisting only of carbon, oxygen, sulfur, and hydrogen atoms, and having from 1 to 5,000, 1 to 500, 1 to 200, 1 to 100, or, e.g., 1 to 20 carbon atoms; or

organic groups consisting only of carbon, oxygen, sulfur, nitrogen and hydrogen atoms and having from 1-5000, 1 to 500, 1 to 200, 1 to 100, or, e.g., 1 to 20 carbon atoms.

In the Formulas, R^{C} denotes a “core group,” that is, an organic group which forms the core of the valency platform, and to which one or more organic groups is attached. In one embodiment, the valency of the core group corresponds to y. If y is 1, then R^{C} is monovalent; if y is 2, then R^{C} is divalent; if y is 3, then R^{C} is trivalent; if y is 4, then R^{c} is tetravalent, and so on.

R^{c} can be, e.g., alkyl, heteroalkyl, aryl, heteroaryl, and can be, e.g., straight chain, branched or cyclic.

In one embodiment, R^{C} is a hydrocarbyl group (i.e., consisting only of carbon and hydrogen) having from 1-2000, or 1 to 200 carbon atoms, e.g., 1 to 100 carbon atoms, or 1 to 50 carbon atoms. R^{C} may be, for example, linear or branched, for may comprise a cyclic structure. In one embodiment, R^{C} is cyclic. R^{C} may be saturated or fully or partially unsaturated. R^{C} may comprise or be an aromatic structure. In one embodiment, R^{C} is an aromatic group, such as a benzyl group having a valency, for example, of between 1 and 6. R^{C} may be, for example —CH₂—; —CH₂CH₂—; —CH₂CH₂CH₂—; or C(CH₂—)₄. R^{c} further may be, for example, —(CH₂)ₙ—, wherein n is 1 to 20.

In one embodiment, R^{C} is an organic group consisting only of carbon, oxygen, and hydrogen atoms, and having, for example, from 1 to 5,000, 1 to 500, 1-200, 1 to 50, or 1-20 carbon atoms, or e.g., 1 to 10 carbon atoms, or 1 to 6 carbon atoms. R^{c} may be or comprise an alkoxy group. In one embodiment, R^{C} is, comprises or is derived from a polyoxyalkylene group, such as a polyoxyethylene group or polyoxypropylene group. R^{C} may be or comprise a divalent polyoxyalkylene group, such as a divalent polyoxyethylene or polyoxypropylene group. In one embodiment, R^{C} is or comprises a divalent polyoxypropylene group, for example, including about 1-5,000, 1 to 500, 1-200, 1-100 or 1-50 oxypropylene units, or, e.g., 1-20, 1-10, or 1, 2, 3, 4, or 5 oxypropylene units. In another embodiment, R^{C} is or comprises a divalent oxyethylene group, for example including about 1 to 5,000, 1 to 500, 1-200, 1-100 or 1-50 oxyethylene units, or e.g, 1-20, 1-10, or 1, 2, 3, 4, or 5 oxyethylene units.

In one embodiment, R^{C} is:

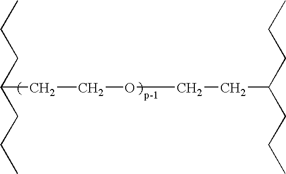

wherein p is a positive integer from 2 to about 500, e.g., 2-200, e.g. 2 to about 50, 2 to about 20, 2 to about 10, or 2 to about 6. In one embodiment, p is 2, 3, 4, 5 or 6.

In one embodiment, R^{C} is an organic group consisting only of carbon, oxygen, nitrogen, and hydrogen atoms, and having from 1 to 5,000, 1 to 500, e.g. 1-200 or 1 to 20 carbon atoms, e.g., 1 to 10 carbon atoms, or 1 to 6 carbon atoms. Examples of such core groups include, but are not limited to those which consist only of carbon, oxygen, nitrogen, and hydrogen atoms.

In one embodiment, RC is an organic group consisting only of carbon, oxygen, sulfur, and hydrogen atoms, and having from 1 to 5,000, 1 to 500, or 1 to 200 carbon atoms, e.g. 1 to 100 carbon atoms, or i to 10 carbon atoms.

R^{c} may be, for example, a C1-200 hydrocarbon moiety; a C1-200 alkoxy moiety; or a C1-200 hydrocarbon moiety comprising an aromatic group.

R^{c} may be or comprise an alcohol containing core compounds having two hydroxyl groups, such as ethylene glycol, diethylene glycol (also referred to as DEG), triethylene glycol (also referred to as TEG), tetraethylene glycol, pentaethylene glycol, hexaethylene glycol, polyethylene glycol (also referred to as PEG), where n is typically from 1 to about 200, and 1,4-dihydroxymethylbenzene. Examples of alcohol containing core compounds having three hydroxyl groups include phluoroglucinol (also known as 1,3,5-trihydroxybenzene), 1,3,5-trihydroxymethylbenzene, and 1,3,5-trihydroxycyclohexane. Examples of alcohol containing core compounds having four hydroxyl groups include pentaerythritol.

In the Formulas, G₂ can denote an organic “linker group.” G₂ in one embodiment is or comprises an organic group, such as alkyl, heteralkyl, aryl, or heteroaryl, and may be, or may contain, e.g., a straight chain, branched or cyclic structure. G₂ may, for example, comprise hydrocarbyl, ethyleneoxy, polyethyleneoxy, propyleneoxy or polypropyleneoxy groups, or combinations thereof. G₂ optionally may comprise other heteroatoms including S and N.

G₂ also may comprise functional groups such as amine, amide, ester, ether, ketone, aldehyde, carbamate and thioether. G₂ also may comprise functional groups such as primary secondary and tertiary, saturated or unsaturated alkyl amine groups, such as piperazinyl or piperidinyl groups. G₂ also may comprise functional groups including polyalcohol, polyamine; polyether; hydrazide; hydrazine; carboxylic acid; anhydride; halo; sulfonyl; sulfonate; sulfone; imidate; cyanate; isocyanate; isothiocyanate; formate; thiol; alcohol; oxime; imine; aminooxy; and maleimide.

In one embodiment, G₂ is a hydrocarbyl group (i.e., consisting only of carbon and hydrogen) comprising 1 to 5,000, 1 to about 500 or 1 to about 200 carbon atoms, e.g., 1 to 100 carbon atoms, or 1 to 10 carbon atoms. In one embodiment, G₂ is or comprises an alkyl group, e.g., —(CH₂)_{q}— wherein q is 1 to 20. In one embodiment, G₂ is or comprises a linear, branched, or cyclic structure. G₂ may be fully or partially unsaturated or saturated. In one embodiment, G₂ comprises an aromatic structure. In one embodiment, G₂ is aromatic. In one embodiment, G₂ is divalent. In one embodiment, G₂ is or comprises —(CH₂)_{q}— wherein q is from 1 to about 20, e.g., 1 to about 10, or 1 to about 6, or 1 to about 4. In one embodiment, G¹ is —CH₂—. In one embodiment, G₂ is or comprises —CH₂CH₂—. In one embodiment, G₂ is or comprises —CH₂CH₂CH₂—.

In one embodiment, G₂ is an organic group consisting only of carbon, oxygen, and hydrogen atoms, and having from 1 to 5,000, 1 to 500, 1 to 200, 1 to 50, e.g., 1-20 carbon atoms, or e.g., from 1 to 10 carbon atoms, or from 1 to 6 carbon atoms. In one embodiment, G₂ is derived from a polyoxyalkylene group. In one embodiment, G₂ is or comprises a divalent polyoxyalkylene group. In one embodiment, G₂ is or comprises a divalent polyoxyethylene group. In one embodiment, G₂ is a divalent polyoxypropylene group. In one embodiment, G₂ is or comprises:

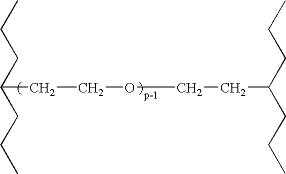

wherein p is from 2 to about 200 or 500, e.g., from 2 to about 50, or from 2 to about 20, or from 2 to about 10, or from 2 to about 6. In one embodiment, p is 2, 3, 4, 5 or 6.

In one embodiment, G₂ is an organic group consisting only of carbon, oxygen, nitrogen, and hydrogen atoms, and having from 1 to 5,000, 1 to 500, e.g., 1 to 200 carbon atoms, e.g., from 1 to 100 carbon atoms, or from 1 to 10 carbon atoms.

G₂ may be, for example, a C₁₋₂₀₀ hydrocarbon moiety; a C₁₋₂₀₀ alkoxy moiety; or a C₁₋₂₀₀ hydrocarbon moiety comprising an aromatic group.

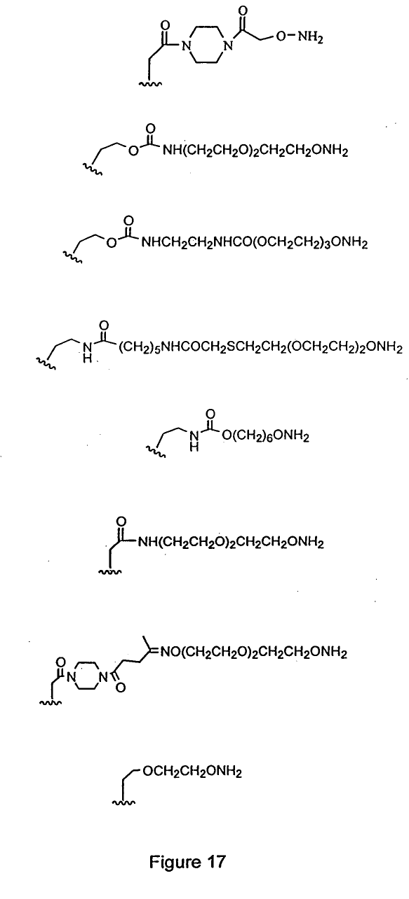

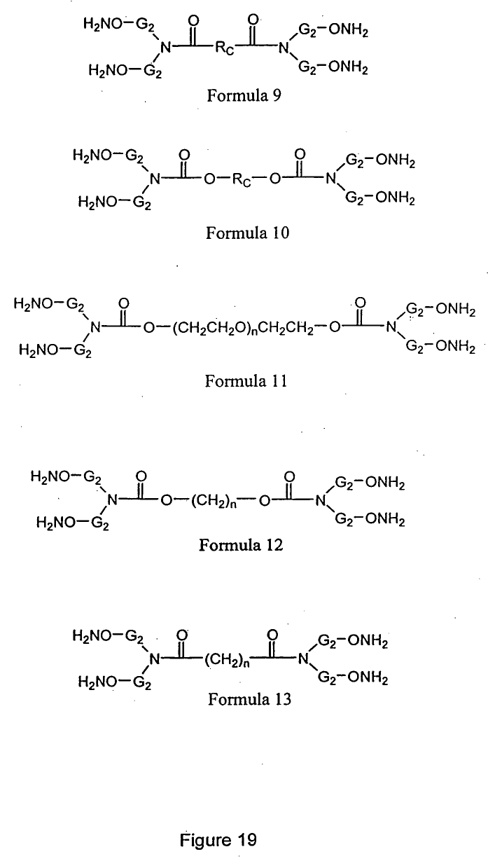

In one embodiment the valency platform molecules have any one of the Formulas 9-13 shown in FIG. 19. In Formulas 9-13, in one embodiment, R^{c} and G₂ are as defined above, and n is about 1-500, e.g., 1-200, 1-100, or 1-50, e.g., 1-20, 1-10, or e.g., 1, 2, 3, 4 or 5. In one embodiment, G₂—ONH₂ has any of the structures shown in FIG. 17.

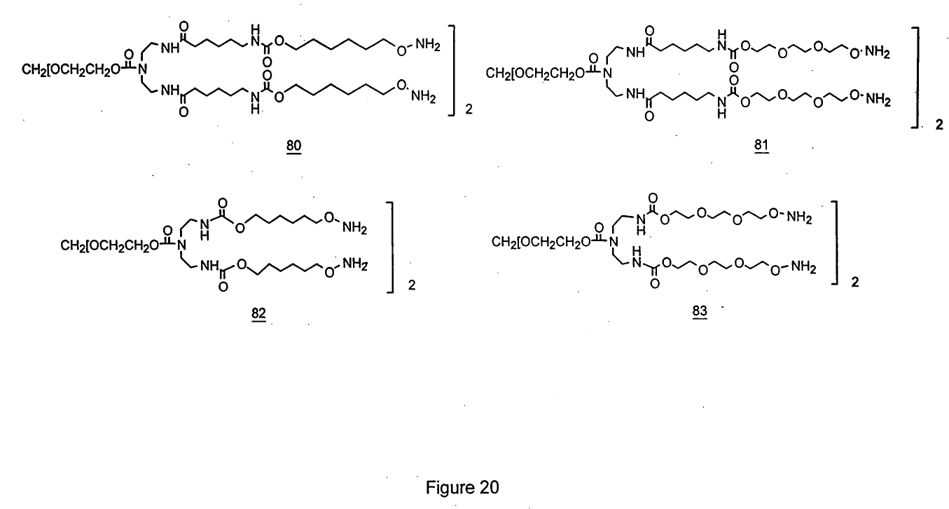

In a further embodiment the valency platform molecules have any of the structures shown in FIGS. 20, 21 and 22.

In one preferred embodiment of each of the compounds and formulas disclosed herein, the valency platform molecule comprises aminooxy groups that are aminooxyalkyl groups, e.g., —CH₂CH₂ONH₂.

Preparation of Molecules Comprising Aminooxy Groups

A variety of molecules may be modified to comprise reactive aminooxy groups as disclosed herein. For example, a wide variety of polymers, such as poly(alkyleneoxide) polymers, including poly(ethyleneoxide) polymers, and in particular, polyethylene glycols, having a molecular weight, for example, less than 10,000 Daltons, can be modified to contain aminooxy groups.

Other molecules that can be modified to include aminooxy groups include branched, linear, block, and star polymers and copolymers, for example those comprising poly(alkyleneoxide) moieties, such as poly(ethylene oxide) molecules. In a preferred embodiment, polyethylene glycol molecules are provided that include at least three aminooxy groups, and optionally have a molecular weight less than about 10,000.

In one aspect, valency platform molecules may be modified to comprise aminooxy groups. Methods for making valency platform molecules are described, for example, in U.S. Pat. Nos. 5,162,515; 5,391,785; 5,276,013; 5,786,512; 5,726,329; 5,268,454; 5,552,391; 5,606,047; 5,663,395 and 5,874,409, as well as in U.S. Ser. No. 60/111,641 and PCT Application No. PCT/US97/10075; published as PCT Publication No. WO 97/46251, Dec. 11, 1997.

Methods known in the art for making valency platform molecules, include, for example, a propagation method, or segmental approach. Such methods can be modified, using the appropriate reagents, to provide aminooxy groups on the resulting molecule. For example, reactive groups, such as halide groups or hydroxy groups may be reacted to attach molecules, such as linkers, that comprise aminooxy groups that are optionally protected. Exemplary methods are demonstrated in the Examples herein.

The valency platforms can be prepared from a segmental approach in which segments are independently synthesized and subsequently attached to a core group. An alternative to the segmental approach is the core propagation process which is an iterative process that may be used to generate a dendritic structure.

Examples of core compounds include alcohol containing core compounds methanol, ethanol, propanol, isopropanol, and methoxypolyethylene glycol, mono-hydroxylamines, ethylene glycol, diethylene glycol, triethylene glycol, tetraethylene glycol, pentaethylene glycol, hexaethylene glycol, 1,4-bis-(hydroxymethyl)benzene and polyethylene glycol HO(CH₂CH₂O)ₙH, wherein, for example, n is about 1-500 or 1-200, e.g., 1-10, or 1 to 5, or primary or secondary amines having two hydroxyl groups.

Aminooxy platforms can be prepared for example to provide a valence of four. Valency Platform molecules of Formula 2 may be prepared as demonstrated in the Examples, e.g., in Example 9. The molecules may be prepared from a tetravalent valency platform molecule with terminal groups which can be converted to aminooxy groups. In general, good leaving groups such as halide or sulfonate, which can be displaced with the oxygen of a protected hydroxylamine derivative, can be used. Also hydroxyl groups can be converted to aminooxy groups using oxaziridine type reagents or Mitsunobu chemistry. In this example a tetra-alkyl halide platform is prepared, and the halide is displaced with the oxygen atom of N-(tert-butyloxycarbonyl)hydroxylamine. Removal of the Boc (N-(tert-butyloxycarbonyl)) protecting groups provides an aminooxy platform.

Other examples involve preparing a suitably protected alkoxyamine bifunctional linker which is attached to the terminal group of a platform. Valency platform molecules of Formula 3 may be prepared by methods described in the Examples, for example, as described in Example 3, from a valency platform molecule which terminates in hydroxyl groups. The hydroxyl groups are converted to an activated carbonate. A bivalent linker is prepared which has a free amino group and a protected aminooxy group. The linker is joined to the platform by reaction of the free, amino group with the carbonate ester to form a carbamate linkage, and the protecting group is removed from the aminooxy group to liberate the aminooxy platform.

Valency platform molecules of Formula 4 may be made, for example, via methods described in detail in the Examples, e.g. in Example 7, from a valency platform molecule that terminates in carboxyl groups. A bivalent linker is prepared which has a free amino group and a protected aminooxy group. The carboxyl groups are activated, and the linker is joined to the platform by reaction of the free amino group with the activated carboxyl group to form an amide linkage. The protecting group is removed from the aminooxy group to liberate the aminooxy platform.

Valency platform molecules of Formula 5 may be made, for example, via methods described in detail in the Examples, e.g., as described in Example 2, from a valency platform molecule that terminates in amino groups. A bivalent linker is prepared which has an activated carboxyl group and a protected aminooxy group. The amino groups on the platform are reacted with the activated carboxyl group on the linker to form an amide linkage. The protecting group is removed from the aminooxy group to liberate the aminooxy platform.

Valency platform molecules of Formula 6 may be made, for example, via methods described in detail in the Examples, e.g. as described in Examples 4 and 6, from a valency platform molecule that terminates in amino groups. A bivalent linker is prepared which has an activated carbonate group and a protected aminooxy group. The amino groups on the platform are reacted with the activated carbonate group on the linker to form carbamate linkage. The protecting group is removed from the aminooxy group to liberate the aminooxy platform.

Valency platform molecules of Formula 7 may be made, for example, via methods described in detail in the Examples, e.g. as described in Example 8, from a valency platform molecule that terminates in aldehyde or ketone groups. A bivalent linker is prepared which has two free aminooxy groups. The aldehyde or ketone groups on the platform (ketones in example 8) are reacted with an excess of the bivalent bis-aminooxy linker to provide the aminooxy platform.

Valency platform molecules of Formula 8 may be made, for example, via methods described in detail in the Examples, e.g. as described in Examples 5a and 5b from a valency platform molecule that terminates in alkyl halide groups. In the examples provided, reactive haloacetyl groups are used. A bivalent linker is prepared which has a free thiol and a protected aminooxy group. The halides (or other suitable leaving groups) on the platform are reacted with the free thiol on the linker to form a thioether linkage. The protecting group is removed from the aminooxy group to liberate the aminooxy platform.

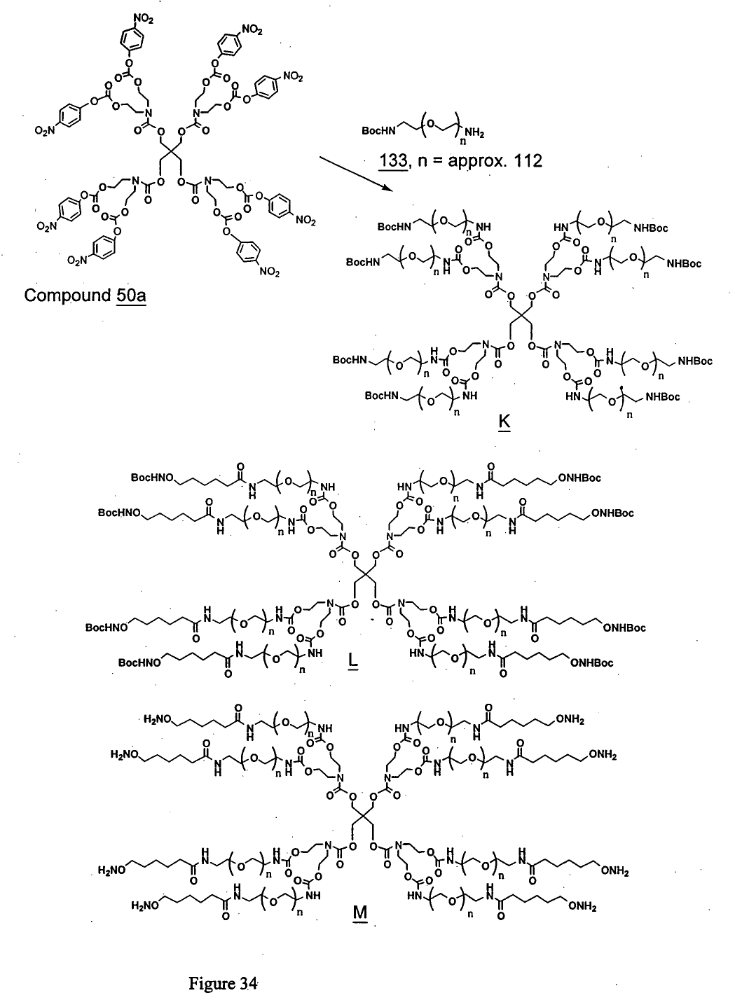

As shown in FIG. 34, in one embodiment a bPEG 8-mer platform, M is synthesized by a process wherein a tetrameric PNP carbonate ester (compound 50a) is reacted with compound 133 resulting in the formation of compound K. The Boc-protecting groups are removed from compound K, and the resulting octa-amine is treated with compound 106 resulting in the formation of compound L. Removal of the Boc-protecting groups from compound M results in the formation of compound M.

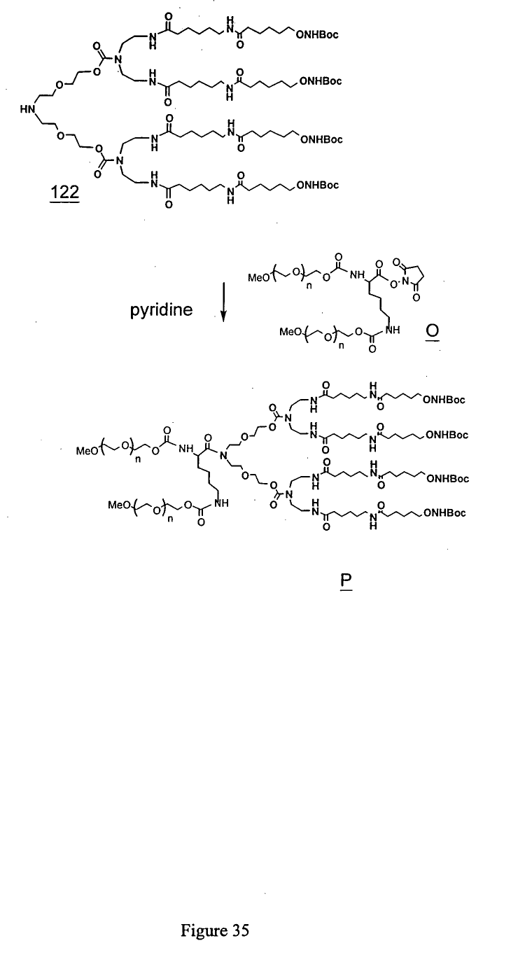

In another embodiment, a tetravalent aminooxy platform with two PEG chains attached is synthesized as shown in FIG. 35 from intermediate 122 which has two PEG chains attached. Thus compound 122 is reacted with NHS ester O (Shearwater Polymers) to form platform P. “PEG” or “polyethylene glycol” or “polyethylene oxide” are used interchangably herein to refer to polymers of ethylene oxide.

Conjugates, Methods of Preparation, and Uses Thereof

Aminooxy groups on molecules such as polyoxyethylene polymers and a variety of valency platform molecules provide reactive groups to which one or more molecules, such as biologically active molecules, may be covalently tethered to form a conjugate.

The term “biologically active molecule” is used herein to refer to molecules which have biological activity, preferably in vivo. In one embodiment, the biologically active molecule is one which interacts specifically with receptor proteins. The biologically active molecule may be, e.g., a polypeptide or a nucleic acid. Depending on the valency of the platform, the platform molecule conjugate may include 1, 2, 3, 4, 5, 6, 7, 8, 9, 10, 11, 12 or more biologically active molecules, or e.g., 16, 18, 32, 36 or more.

Conjugates may be used in a method for treating an antibody mediated disease or other condition in an individual in need of such treatment comprising administering to the individual an effective amount of the conjugates. Conjugates also may be used in a method of inducing specific B cell anergy to an immunogen in an individual comprising administering to the individual an effective amount of the conjugates. The conjugates also may be used in a method of treating an individual for an antibody-mediated pathology in which undesired antibodies are produced in response to an immunogen comprising administering to the individual an effective amount of the conjugates.

In one embodiment, it is preferred that the total molecular weight of the conjugate is no greater than about 200,000 Daltons, for example, in order for the conjugate to be effective as a functional toleragen.

In one embodiment, the biologically active molecule is a domain 1 polypeptide of β2GPI, as described, e.g., in U.S. Ser. No. 60/103,088; in U.S. Ser. No. 09/328,199, filed Jun. 8, 1999; and in PCT Application No. PCT/US99/13194, filed Dec. 16, 1999; now PCT Publication No. WO 99/64595, published Dec. 16, 1999, the disclosures of which are incorporated herein. The domain 1 conjugates can be used in methods for detection of a β₂GPI-dependent antiphospholipid antibody (or an antibody that specifically binds to a domain 1 β₂GPI polypeptide(s)) in a sample by contacting antibody in the sample with the conjugate under conditions that permit the formation of a stable antigen-antibody complex; and detecting stable complex formed if any. The conjugates also can be used in methods of inducing tolerance in an individual which comprise administering an effective amount of a conjugate to an individual, particularly a conjugate comprising a domain 1 β₂GPI polypeptide(s) that lacks a T cell epitope, wherein an effective amount is an amount sufficient to induce tolerance.

In another embodiment, there is provided a conjugate of a valency platform molecule and at least one αGal epitope or analog thereof that specifically binds to an anti-αGal antibody. In another aspect, a method of reducing circulating levels of anti-αGal antibodies in an individual is provided comprising administering an effective amount of the conjugate to the individual, wherein an effective amount is an amount sufficient to reduce the circulating levels of anti-αGal antibodies, or to neutralize circulating levels of anti-αGal antibodies. In another aspect, a method of inducing immunological tolerance (generally to a xenotransplantation antigen, more specifically to αGal), is provided, the method comprising administering an effective amount of the conjugate comprising the αGal epitope or analog thereof. The conjugates also can be used to detect the presence and/or amount of anti-αGal antibody in a biological sample. Methods of performing a xenotransplantation in an individual also are provided, comprising administering a conjugate to the individual; and introducing xenotissue to the individual. In another aspect, methods of suppressing rejection of a transplanted tissue are provided comprising comprising administering the conjugate to the individual in an amount sufficient to suppress rejection. These methods are described generally in PCT Application No. PCT/US99/29338; published as PCT Publication No. WO 00/34296, Jun. 15, 2000.

The conjugates also may be used for immunotolerance treatment of lupus optionally based on assessment of initial affinity of antibody from the individual (i.e., antibody associated with lupus, namely, anti double stranded DNA antibodies) and used as a basis for selecting the individual for treatment, or in methods of identifying individuals suitable (or unsuitable) for treatment based on assessing antibody affinity. Methods of treating systemic lupus erythematosus (SLE) in an individual comprise administering to the individual a conjugate comprising (a) a non-immunogenic valency platform molecule and (b) two or more polynucleotides which specifically bind to an antibody from the individual which specifically binds to double stranded DNA. These methods are described generally in PCT Application No. PCT/US99/29336; published as PCT Publication No. WO 00/33887, Jun. 15, 2000.

Thus, the valency platform may be covalently linked to form a conjugate with one or more biologically active molecules including oligonucleotides, peptides, polypeptides, proteins, antibodies, saccharides, polysaccharides, epitopes, mimotopes, enzymes, hormones and drugs, lipids, fatty acids, or mixtures thereof to form a conjugate.

The terms “protein”, “polypeptide”, and “peptide” are used interchangeably herein to refer to polymers of amino acids of any length. The polymer may be linear or branched, it may comprise modified amino acids, and it may be interrupted by non-amino acids. It also may be modified naturally or by intervention; for example, disulfide bond formation, glycosylation, myristylation, acetylation, alkylation, phosphorylation or dephosphorylation. Also included within the definition are polypeptides containing one or more analogs of an amino acid (including, for example, unnatural amino acids) as well as other modifications known in the art.

One advantage of the conjugates of valency platforms and other molecules comprising aminooxy groups is the ability to introduce enhanced affinity of the tethered biologically active molecules for their binding partners, for example when the binding partners are associated in a cluster. The covalent attachment of plural biological molecules to the valency platform molecule provides an enhanced local concentration of the biomolecules as they are associated together for example on the platform molecule. Another advantage of the valency platforms is the ability to facilitate binding of multiple ligands, as is useful in B cell tolerance. For example, the conjugates can be used as toleragens to present multivalent epitopes to induce clustering on the surface of a B cell. Another advantage of the valency platforms is the ability to include functionality on the “core” that can be independently modified to enable the preparation of conjugates which can be tailored for specific purposes.

In general a molecule comprising an aminooxy group is reacted with a second molecule comprising a carbonyl group, such as an aldehyde or ketone, to form an oxime conjugate. The second molecule may be modified to contain the reactive aldehyde or ketone. The oxime bond can be further modified. For example, it may be converted to an aminooxy bond via reduction or reaction with nucleophiles by known methods to form an aminooxy conjugate.

In one embodiment, a method of preparing chemically defined multivalent conjugates of native polypeptides or proteins with multivalent preferably non-immunogenic valency platform molecules comprising aminooxy groups is provided, wherein, if needed, the polypeptide is selectively modified to generate an aldehyde or ketone moiety at a specific position on the polypeptide. The polypeptide then is reacted with the multivalent valency platform molecule which contains aminooxy groups to form one or more oxime linkages between the platform and the polypeptide.

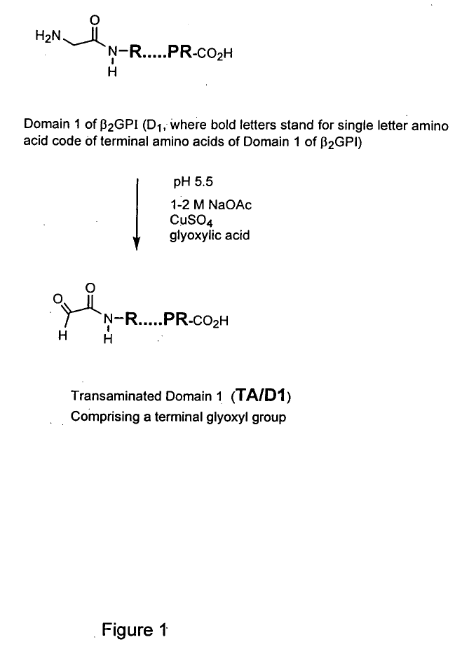

Amines, for example at the N-terminus, of virtually any polypeptide or other molecule can be converted to an aldehyde or a ketone by a reaction which is known in the art as a transamination reaction. Essentially, the transamination reaction converts the carbon-nitrogen single bond to a carbon oxygen double bond. For example, a glycine at the N-terminus can react to form a glyoxyl group, an aldehyde, as shown in FIG. 1. Most other amino acids react to form a ketone by virtue of the amino acid side chain.

Another way to generate an glyoxyl group at the N-terminus is to oxidize an N-terminal serine or threonine with sodium periodate. This oxidation cleaves the carbon-carbon bond between the hydroxyl and amino groups of the N-terminal serine or threonine providing a glyoxyl group. Thus in one embodiment, polypeptides can be site specifically modified by forming a ketone or aldehyde at the N-terminus. Synthetic polypeptides and other drugs or biologically active molecules can be modified similarly to include aldehydes or ketones which can be used to form oxime linkages.

Multivalent platforms containing aminooxy reactive groups permit covalent attachment of the selectively modified polypeptides to the platforms. The valency platform molecule may comprise, e.g., aminooxyacetyl groups or aminooxyalkyl groups.

As used herein, an “aminooxyacetyl group” refers to an aminooxy group with an alpha carbonyl, such as —COCH₂—ONH₂, while an “aminooxyalkyl group” refers to an aminooxy group on a first carbon, wherein the first carbon is preferably not directly attached to an electron withdrawing group, such as a second carbon which is part of a carbonyl group. One preferred aminooxyalkyl group is —CH₂—CH₂—ONH₂. Other embodiments of aminooxyalkyl groups include —CH(OH)CH₂ONH₂, and —CH₂CH(CH₃)ONH₂.

Aminooxyacetyl (AOA) groups can be attached to multivalent platforms containing amine groups by acylation with a N-protected aminooxyacetyl group followed by protecting group removal. Reaction of glyoxyl polypeptides with aminooxyacetyl groups proceeds slowly to form oxime linkages between the polypeptide and the aminooxy functionalized platform. The long reaction times necessary for the reaction can permit competing side reactions to occur. N-terminal α-keto-amides, which are formed with the transamination of N-terminal amino acids other than glycine, react even more slowly or not at all to make multivalent conjugates.

Aminooxyalkyl groups (AO alkyl groups) are preferred and react more readily with ketones and aldehydes to form oximes than aminooxyacetyl groups. An aminooxy group on an alkyl chain (for example, a triethylene glycol chain) is, for example, more than ten times more reactive in forming oximes than an analogous aminooxyacetyl group. The aminooxyacetyl group is generally less reactive than other aminooxy groups (aminooxyalkyl groups) which are not adjacent to a carbonyl. It is believed that the carbonyl of the aminooxyacetyl group lowers reactivity due to electron withdrawing effects.

In one embodiment, terminal aminooxyalkyl groups that can react with glyoxyl-polypeptides on platforms are provided that are designed with enhanced reactivity toward oxime formation. In one embodiment, the aminooxy groups are provided on triethylene glycol or hexyl chains; however any other chain is possible including those comprising carbon, oxygen, nitrogen or sulfur atoms. In one preferred embodiment, the aminooxy groups in the platform molecule are aminooxyalkyl groups, such as —CH₂CH₂ONH₂.

Examples of attachment of biomolecules with aldehyde or ketone functionality to aminooxy platforms via oxime bond formation are provided in the Examples. Examples 10 and 11 describe how transaminated polypeptides, or polypeptides otherwise modified with aldehyde or ketone groups, are reacted with aminooxy platforms. In these cases transaminated Domain 1 is attached to tetravalent platforms by treating the platforms with the glyoxyl-polypeptide in acidic aqueous solution. A preferred acidic condition is 100 mM pH 4.6 sodium acetate. In the case of making a tetravalent Domain 1 conjugate, an excess of four equivalents, for example six equivalents, of transaminated Domain 1 is used. Aminooxyalkyl reactive groups are more reactive than aminooxyacetyl groups, allowing the reaction to take place more readily with the opportunity for fewer byproducts. Example 10 describes conjugate formation with an aminooxyacetyl platform. Example 11 describes conjugate formation with an aminooxyalkyl platform.

Two alternative methods of preparing tetravalent Domain 1 conjugates are shown in Examples 13 and 14. Both of these examples involve attaching a linker to transaminated Domain 1 via an oxime bond, then using the linker to attach to a platform with suitable reactive groups. The advantage of attaching the linker to transaminated Domain 1 first is that excess linker can be added to drive the oxime forming reaction to completion.

Example 13 describes how a bis-aminooxy linker is attached to Domain 1 first, then the polypeptide with aminooxy linker attached is reacted with a ketone derivatized platform to provide the desired tetravalent conjugate.

Example 14 demonstrates how a heterobifunctional linker can be used to attach a thiol linker to Domain 1 via an oxime bond. Domain 1 with the thiol linker attached is then reacted with a reactive alkyl halide platform to provide a tetravalent conjugate.

It is apparent that the conjugates formed in Examples 13 and 14 are the same conjugates which would be formed if the linkers were attached first to the platform, followed by conjugation with transaminated Domain 1.

The disclosures of all publications, patents, patent applications and published patent applications referred to herein by an identifying citation are hereby incorporated herein by reference in their entirety.

The invention will be further understood by the following nonlimiting examples.

### EXAMPLES

In the following examples, the following abbreviations are used: DCC, 1,3-dicyclohexylcarbodiimide; DIC, 1,3-diisopropylcarbodiimide; DBU, 1,8-diazabicyclo[5.4.0]undec-7-ene; NHS, N-hydroxysuccinimide; HOBt, 1-hydroxybenzotriazole; DMF, dimethylformamide;

### Example 1

### Transamination of Domain 1

Synthesis of transaminated domain 1 (ta/d1): Water and sodium acetate buffer were sparged with helium before use. The domain 1 polypeptide of β2GPI was used, which is described in U.S. Ser. No. 60/103,088, filed Oct. 5, 1998; in U.S. Ser. No. 09/328,199, filed Jun. 8, 1999; and in PCT Application No. PCT/US99/13194; published as PCT Publication No. WO 99/64595, Dec. 16, 1999, the disclosures of which are incorporated herein. The Domain 1 polypeptide, as illustrated in FIG. 1, has an N-terminal glycine. Domain 1 (10.55 mg, 1.49 μmol) was dissolved in 0.5 mL of H₂O in a polypropylene tube, and 4.0 mL of 2 M pH 5.5 NaOAc buffer was added. A solution of 3.73 mg (14.9 μmol) of CUSO₄ in 0.5 mL of H₂O was added to the mixture, followed by a solution of 2.75 mg (29.9 μmol) of glyoxylic acid in 0.5 mL of 2 M pH 5.5 NaOAc buffer. The mixture was kept under nitrogen atmosphere and agitated gently for 18 h at which time the reaction appeared complete by analytical HPLC using a 4.6 mm×250 mm, 300 Å, 5 μm, diphenyl column (Vydac, Hesperia, Calif.) with detection at 280 nm (1 mL/min; gradient 25%-45% B, 0-20 min, A=0.1% TFA/H₂O, B=0.1% TFA/CH₃CN). Approximate retention times are as follows: D1, 13.2 min; TA/D1, 13.7 min; oxidized TA/D1, 13.4 min). The mixture was diluted to a volume of 20 mL with 0.1% TFA/H₂O, filtered, and purified by HPLC (22.4 mm×250 mm, 300 Å, 10 μm, diphenyl column (Vydac) (12 mL/min; gradient 25%-40% B, 0-40 min, A=0.1% TFA/H₂O, B=0.1% TFA/CH₃CN). Fractions containing pure TA/D1, as evidenced by analytical HPLC, were pooled and lyophilized to provide 5.0 mg (48%) of TA/D1. The reaction scheme is shown in FIG. 1.

### Example 2

### Synthesis of an Aminooxyacetyl/PITG Platform

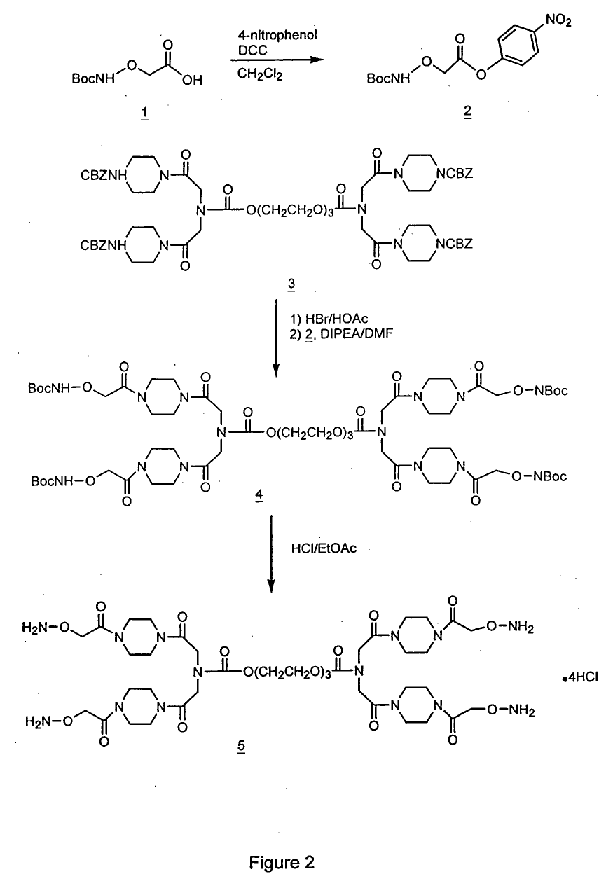

The synthetic scheme is shown in FIG. 2.

4-Nitrophenyl-N-(tert-butyloxycarbonyl)aminooxyacetate, 2: To a stirred solution of 1.5 g (7.85 mmol) of N-(tert-butyloxycarbonyl)aminooxyacetic acid (Aldrich Chemical Co., St. Louis, Mo.), compound 1, in 35 mL of anhydrous THF at 0° C. was added 1.09 g (7.85 mmol) of 4-nitrophenol followed by 1.62 g (7.85 mmol) of DCC. The mixture was stirred under a nitrogen atmosphere for 0.5 h at 0° C. and at room temperature for 18 h. The mixture was filtered to remove dicyclohexylurea, and the filtrate was concentrated and purified by silica gel chromatography (95/5 CHCl₃/isopropyl alcohol) to give 2.30 g (94%) of compound 2 as a white solid: ¹H NMR (CDCl₃) δ 1.51 (s, 9H), 4.73 (s, 2H), 7.36 (d, 2H), 7.73 (s, 1H), 8.32 (d, 2H).

Synthesis of Boc-protected aminooxyacetyl/PITG Platform, 4: Compound 3 (300 mg, 0.235 mmol (prepared as described in PCT Application No. PCT/US97/10075; published as PCT Publication No. WO 97/46251, Dec. 11, 1997) was treated with 1.5 mL of a 30% solution of HBr in acetic acid for 30 min. The HBr salt of the resulting tetra-amine was precipitated by addition of diethyl ether. The mixture was centrifuged, and the supernatant was removed and discarded. The remaining solid was washed with ether, dried under vacuum, and dissolved in 9 mL of DMF. To the resulting mixture was added 294 μL (1.69 mmol) of diisopropylethylamine followed by a solution of 410 mg (1.31 mmol) of compound 2 in 3 mL of DMF. The mixture was stirred under nitrogen atmosphere for 4 h and partitioned between 15/1 CHCl₃/MeOH and brine. The aqueous layer was washed twice with 15/1 CHCl₃/MeOH, and the combined organic layers were dried (Na₂SO₄) and concentrated to give 680 mg of an oil. Purification by silica gel chromatography (step gradient 95/5 to 75/25 CHCl₃/MeOH) gave 215 mg (65%) of compound 4 as a white solid: ¹H NMR (CDCl₃) δ 1.49 (s, 36H), 3.40-3.73 (m, 40H), 4.24(m, 12H), 4.59 (overlapping singlets, 8H), 8.21 (s, 2H), 8.32 (s, 2H).

Aminooxyacetyl/PITG Platform, Compound 5: HCl gas was bubbled through a stirred solution of 67 mg (0.047 mmol) of compound 4 in 10/1/1 EtOAc/CHCl₃/MeOH for 15 min, and the mixture was stirred for an additional 15 min. The mixture was concentrated under vacuum and kept under vacuum for 16 h to provide 43 mg (78%) of compound 5 as a white solid: ¹H NMR (DMSO) δ 3.33-3.67 (m, 40H), 4.08 (m, 4H), 4.18 (s, 8H), 4.90 (s, 8H); mass spectrum (ES) m/z calculated for C₄₀H₆₉N₁₄O₁₈ (M+H): 1033. Found: 1033.

### Example 3

### Synthesis of AOTEG/DEA/DEG Platform

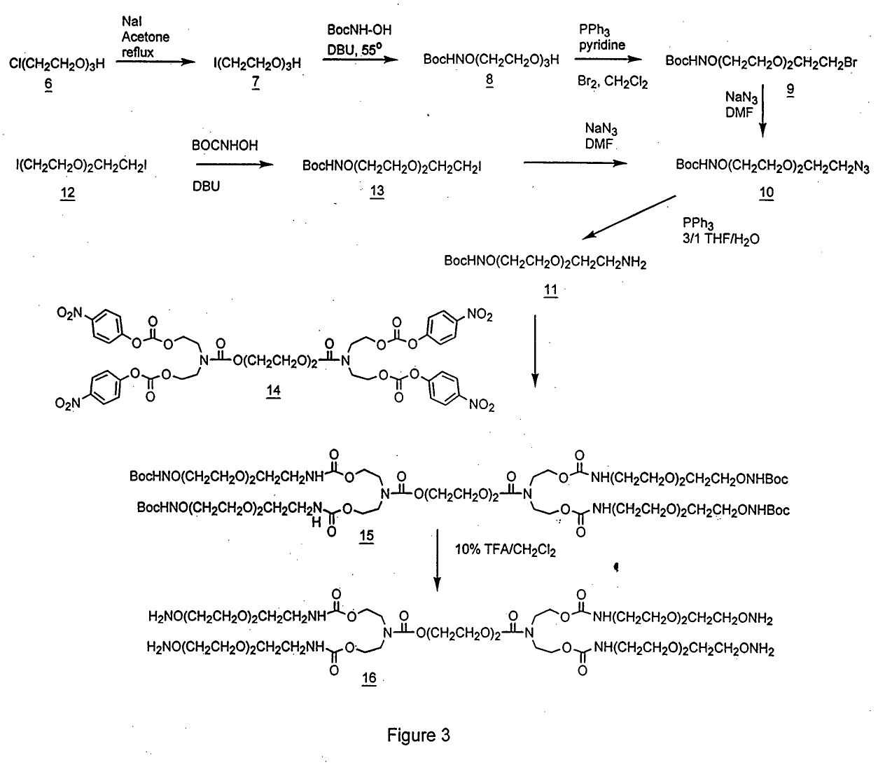

The synthetic scheme is shown in FIG. 3.

2-[2-(2-iodoethoxy)ethoxy]ethanol, 7: 2-[2-(2-Chloroethoxy)ethoxy]ethanol (Aldrich Chemical Co.) (12.66 g, 75.1 mmol) and sodium iodide (33.77 g, 225.3 mmol) were dissolved in 250 mL of acetone. A reflux condensor was attached to the flask, and the mixture was heated at reflux for 18 h. When cool, the mixture was concentrated, and the residue was shaken with 400 mL of CH₂Cl₂ and a mixture of 300 mL of water and 100 mL of saturated aqueous sodium bisulfite solution. The aqueous layer was washed twice with 400 mL portions of CH₂Cl₂, and the combined CH₂Cl₂ layers were dried (MgSO₄), filtered, and concentrated to provide 18.3 g (94%) of 7 as a light yellow oil which was used in the next step without further purification: ¹H NMR (CDCl₃) δ 2.43 (brd s, 1H), 3.28 (t, 2H), 3.61 (m, 2H), 3.68 (s, 4H), 3.78 (m, 4H); mass spectrum (ES) m/z calculated for C₆H₁₃O₃₁Na (M+Na): 283.0. Found: 283.0.

2-[2-(2-N-(tert-butyloxycarbonyl)aminooxyethoxy)ethoxy]ethanol, 8: To 5.85 g (1:50 mmol) of 2-[2-(2-iodoethoxy)ethoxy]ethanol, compound 7, was added 2.00 g (1.00 mmol) of N-(tert-butyloxycarbonyl)hydroxylamine (Aldrich Chemical Co.) and 3.36 mL (3.42 g, 1.50 mmol) of DBU. The mixture was stirred to give a viscous liquid that became hot to the touch and placed in a 55° C. oil bath for 18 h resulting in the formation of a white precipitate which solidified the mixture. The mixture was dissolved in 20 mL of CH₂Cl₂ and added to 500 mL of stirred EtOAc resulting in the formation of a precipitate which was removed by filtration, and the filtrate was concentrated to give a brown-yellow oil. Purification by flash chromatography (50% acetone/hexane) to give 2.61 g (67%) of 8 as an oil: ¹H NMR (CDCl₃) δ 1.50 (s, 9H), 3.65 (t, 2H), 3.70 (brd s, 4H), 3.76 (m, 4H), 4.06 (t, 2H), 7.83 (brd s, 1H); ¹³C NMR (CDCl₃) δ 28.0, 61.3, 68.9, 70.1, 70.3, 72.5, 72.6, 75.1, 81.2, 157.1.

2-[2-(2-N-(tert-butyloxycarbonyl)aminooxyethoxy)ethoxy]ethylbromide, compound 9: Bromine (approximately 0.283 mmol) was added dropwise to a solution of 50 mg (0.188 mmol) of compound 8, 74 mg (0.283 mmol) of triphenylphosphine, and 31 μL (30 mg, 0.377 mmol) of pyridine in 2 mL of CH₂Cl₂ until an orange color persisted. The mixture was stirred at room temperature for 0.5 h, and 1 mL of a saturated solution of sodium bisulfite was added to quench excess bromine. The mixture was then partitioned between 10 mL of H₂O and 2×15 mL of EtOAc. The combined organic layers were washed with brine, dried (Na₂SO₄), filtered, and concentrated. Purification of the residue by silica gel chromatography (35/65 acetone/hexane) provided 54 mg of compound 9 as an oil: ¹H NMR (CDCl₃) δ 1.49 (s, 9H), 3.48 (t, 2H), 3.68 (s, 4H), 3.73 (m, 2H), 3.84 (t, 2H), 4.03 (t, 2H), 7.50 (s, 1H); ¹³C NMR (CDCl₃) δ 28.3, 30.4, 69.4, 70.6 (two signals), 71.3, 75.5, 81.7, 156.9.

2-[2-(2-N-(tert-butyloxycarbonyl)aminooxyethoxy)ethoxy]ethylazide, 10: Synthesis from compound 9:

A solution of 100 mg (0.305 mmol) of compound 9 in 0.25 mL of anhydrous DMF was added to a solution of 159 mg (2.44 mmol) of sodium azide in 0.5 mL of anhydrous DMF. An additional 0.25 mL of DMF was used to rinse residual 9 into the reaction mixture, and the mixture was heated at 115° C. for 3 h. When cool, the mixture was partitioned between 3 mL of H₂O and 4×3 mL of CH₂Cl₂. The combined organic layers were washed with 10 mL of H₂O, dried (Na₂SO₄), filtered, and concentrated to provide a yellow oil. Purification by silica gel chromatography (35/65 acetone/hexane) gave 67 mg (76%) of 10 as an oil: ¹H NMR (CDCl₃) δ 1.47 (s, 9H), 3.41 (t, 2H), 3.69 (brd s, 4H), 3.73 (m, 4H), 4.03 (t, 2H), 7.50 (s, 1H); ¹³C NMR (CDCl₃) δ 28.1, 50.5, 69.1, 70.1, 70.4 (two signals), 75.2, 81.3, 156.7.

Synthesis of 10 from compound 13: To a solution of 258 mg (0.69 mmol) of compound 13 in 5 mL of DMF under nitrogen atmosphere was added 358 mg (5.50 mmol) of sodium azide. The mixture was stirred for 18 hours at room temperature, 100 mL of water was added, and the mixture was extracted with 3×50 mL of EtOAc. The EtOAc layers were combined and washed with 50 mL of water, dried (Na₂SO₄), filtered, and concentrated to provide 294 mg of a colorless oil. Purification by silica gel chromatography (30/70 acetone/hexanes) provided compound 10 as a colorless oil.

Compound 11: Compound 10 (1.36 g, 4.70 mmol) and triphenylphosphine (1.48 g, 5.64 mmol) were dissolved in 24 mL of THF and 8 mL of H₂O, and the resulting solution was stirred at room temperature for 2 hours. Approximately 160 μL (eight drops) of 1 N NaOH was added, and the mixture was stirred for 18 hours. The mixture was concentrated under vacuum, and the concentrate was purified by silica gel chromatography (80/8/2 CH₃CN/H₂O/con NH₄OH) to give 1.16 g (94%) of 11 as a yellow oil: ¹H NMR (CDCl₃) δ 1.50 (s, 9H), 1.90 (brd, 2H), 2.88 (t, 2H), 3.56 (t, 2H), 3.65 (m, 4H), 3.71 (m, 2H), 4.01 (m, 2H).

1,2-Bis(2-iodoethoxy)ethane, compound 12: A solution of 10.0 g (5.3 mmol) of 1,2-bis(2-chloroethoxy)ethane (Aldrich Chemical Co.) and 16.0 g (107 mmol) of sodium iodide in 110 mL of acetone was heated at relux for 18 h. The mixture was concentrated and the residue was triturated with CHCl₃ to dissolve product while salts remained undissolved. The mixture was filtered, and the filtrate was concentrated to give an orange oil. Purification by silica gel chromatography (step gradient, 10/90 EtOAc/hexanes to 15/85 EtOAc/hexanes) to provide 17.8 g (90%) of an orange oil: ¹H NMR (CDCl₃) δ 3.28 (t, 4H), 3.67 (s, 4H), 3.78 (t, 4H); ¹³C NMR (CDCl₃) δ 3.6, 70.5, 72.2.

Compound 13: DBU (284 μL, 290 mg, 1.90 mol) was added to a mixture of 266 mg (2.0 mmol) of N-(tert-butyloxycarbonyl)hydroxylamine (Aldrich Chemical Co.) and 2.96 g (8.0 mmol) of compound 12, and the mixture was capped and shaken until homogeneous. After 15 minutes the mixture solidified, and it was allowed to stand for 45 minutes. To the mixture was added 5 mL of CH₂Cl₂, and the mixture was shaken again to dissolve solids. The resulting solution was added to 200 mL of EtOAc. An additional 50 mL of EtOAc was added, and the mixture was filtered to remove solids. The filtrate was concentrated to give an oil which was partitioned between 100 mL of EtOAc and 3×50 mL of 1 N HCl solution. The EtOAc layer was washed with 2×50 mL of 1 N NaOH followed by 2×50 mL of 5% sodium bisulfite solution and concentrated to provide 2.6 g of yellow oil. Purification by silica gel chromatography (step gradient, 20/80 to 45/55 EtOAc/hexanes) gave 515 mg (69%) of compound 13 as a yellow oil: ¹H NMR (CDCl₃) δ 1.50 (s, 9H), 3.28 (t, 2H), 3.68 (s, 4H), 3.72 (m, 4H), 4.02 (t, 2H), 7.72 (s, 1H); ¹³C NMR (CDCl₃) δ 2.9, 28.3, 68.9, 69.4, 70.2, 70.6, 72.0, 75.4, 81.6, 156.9.

Diethyleneglycol bis-4-nitrophenylcarbonate, Compound 60: Pyridine (30.5 mL, 377 mmol) was slowly added to a 0° C. solution of 5.0 g (47.11 mmol) of diethylene glycol and 23.74 g (118 mmol) of 4-nitrophenylchloroformate in 500 mL of THF. The cooling bath was removed, and the mixture was stirred for 18 hours at room temperature. The mixture was cooled back to 0° C., acidified with 6 N HCl, and partitioned between 400 mL of 1 N HCl and 2×400 mL of CH₂Cl₂. The combined organic layers were dried (MgSO₄), filtered, and concentrated to give 24.3 g of a white solid. Crystallization from hexanes/EtOAc gave 16.0 g (78%) of compound 60 as a white powder: mp 110° C.; ¹H NMR (CDCl₃) δ 3.89 (t, 4H), 4.50 (t, 4H), 7.40 (d, 4H), 8.26 (d, 4H).

Compound 61: A solution of 2.5 g (5.73 mmol) of compound 60 in 17 mL of pyridine was added to a 0° C. solution of 1.8 g (17.2 mmol) of diethanolamine in 3 mL of pyridine. The cooling bath was removed, and the mixture was stirred for 5 hours at room temperature to yield compound 61, which was not isolated but was used as is in the next step.

Compound 14: The mixture from the previous step was cooled back to 0° C., 40 mL of CH₂Cl₂ was added followed by a solution of 11.55 g (57.3 mmol) of 4-nitrophenylchloroformate in 60 mL of CH₂Cl₂, and the mixture was stirred for 20 hours at room temperature. The mixture was cooled back to 0° C., acidified with 1 N HCl, and partitioned between 300 mL of 1 N HCl and 2×200 mL of CH₂Cl₂. The combined organic layers were dried (MgSO₄), filtered, and concentrated to give 13.6 g of yellow solid. Purification by silica gel chromatography (CH₂Cl₂/MeOH and EtOAc/hexanes) provided 4.91 g (83%) of compound 14 as a sticky amorphous solid: ¹H NMR (CDCl₃) δ 3.72 (m, 12H), 4.31 (t, 4H), 4.48 (m, 8H), 7.40 (m, 8H), 8.29 (m, 8H).

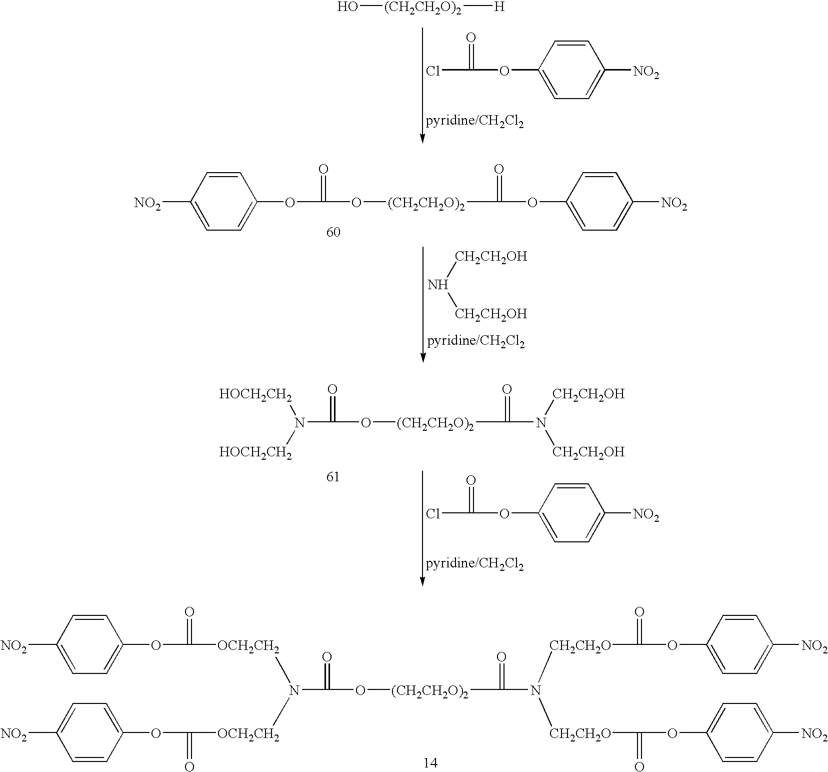

BOC-Protected AOTEG/DEA/DEG Platform, Compound 15:

Triethylamine (157 μL, 114 mg, 1.13 mmol) was added to a stirred solution of 193 mg (0.188 mmol) of compound 14 (prepared as described above and in U.S. Ser. No. 60/111,641, filed Dec. 9, 1998) followed by 298 mg (1.13 mmol) of compound 11. The mixture was allowed to come to room temperature and was stirred overnight. The mixture was cooled to 0° C., acidified with 1 N HCl, and partitioned between 20 mL of 1 N HCl and 4×20 mL of CH₂Cl₂. The combined organic layers were washed with saturated NaHCO₃ solution, dried (MgSO₄), filtered, and concentrated to give 279 mg of yellow oil. Purification by silica gel chromatography (97/3 CH₂Cl₂/MeOH) provided 138 mg (48%) of 15 as an oil: ¹H NMR (CDCl₃) δ 1.49 (s, 36H), 3.35 (m, 8H), 3.46-3.78 (m, 44H), 4.04 (t, 8H), 4.21 (m, 12H), 5.80 (m, 4H), 7.91 (s, 4H); mass spectrum (ES) m/z calculated for C₆₂H₁₁₇N₁₀O₃₃ (M+H): 1528.8. Found: 1528.5.

Compound 16: Compound 15 (60 mg, 39.2 μmol) was dissolved in 10 mL of 1/9 trifluoroacetic acid/CH₂Cl₂, and the mixture was kept at room temperature for 3 h. A gentle stream of nitrogen was used to evaporate the solvent, and the residue was dissolved in a minimal amount of chromatography solvent (5/7.5/87.5 con NH₄OH/H₂O/CH₃CN) which was used to load the mixture onto a silica gel column. Purification by silica gel chromatography (step gradient, 5/7.5/87.5 to May 10, 1985 con NH₄OH/H₂O/CH₃CN) provided 36 mg (82%) of 16 as a colorless oil: ¹H NMR (CDCl₃) δ 3.37 (m, 8H), 3.58 (m, 16H), 3.67 (s, 16H), 3.71 (m, 12H), 3.86 (m, 8H), 4.17-4.29 (m, 12H), 4.93 (brd, 8H), 5.91 (m, 4H); ¹³C NMR (CDCl₃) □ 40.9, 47.7, 48.2, 62.9, 64.7, 69.4, 69.6, 70.2, 70.3, 70.5, 74.8, 156.1, 156.6; mass spectrum (ES) m/z calculated for C₄₂H₈₅N₁₀O₂₅ (M+H): 1129. Found: 1129.

For the purpose of checking purity by analytical HPLC, the tetra-acetone oxime was prepared as follows. Compound 16 (0.38 mg, 0.34 μmol) was dissolved in 240 μL of 0.1 M NaOAc buffer in an HPLC sample vial. To the solution was added 10 μL of a solution of 49 μL of acetone in 2.0 mL of 0.1 M NaOAc buffer. The mixture was allowed to stand for 1 h and an aliquot was analyzed by HPLC (4.6 mm C₁₈ column, 1 mL/min, 210 nm detection, gradient, 10-60% B over 20 min, A=0.1% TFA/H₂O, B=0.1% TFA/CH₃CN, t_{R}=19 min); mass spectrum of collected eluent (ES) m/z calculated for C₅₄H₁₀₁N₁₀O₂₅ (M+H): 1289. Found: 1289.

### Example 4

### Synthesis of AOTEG/PIZ/DEA/DEG Platform

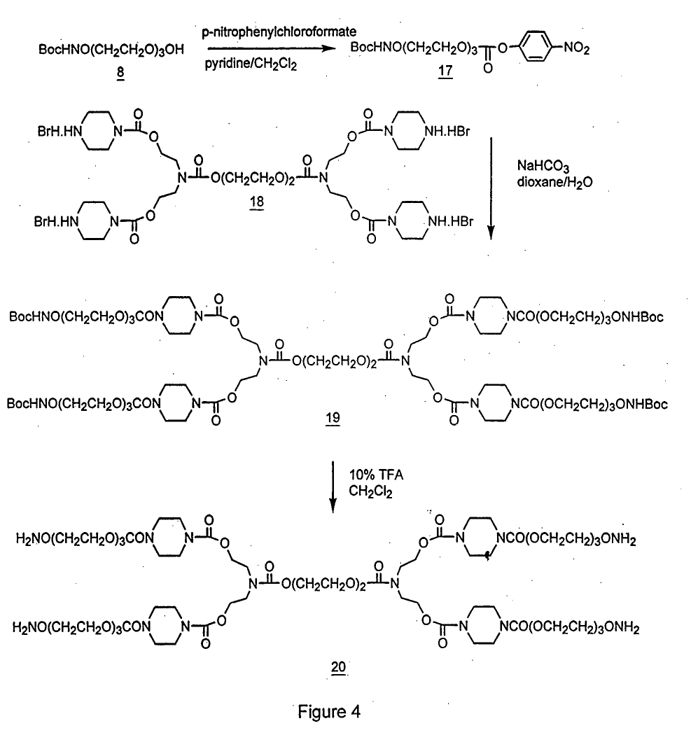

The synthetic scheme is shown in FIG. 4.

Compound 17: Pyridine (610 μL, 596 mg, 7.54 mmol) was added slowly to a stirred solution of 500 mg (1.88 mmol) of compound 8 and 760 mg (3.77 mmol) of p-nitrophenylchloroform ate in 14 mL of CH₂Cl₂, and the mixture was stirred at room temperature for 18 hours. The mixture was cooled to 0° C. and acidified with 1N aqueous HCl. The resulting mixture was partitioned between 100 mL of 1 N aqueous HCl and 3×100 mL of CH₂Cl₂. The combined organic layers were dried (MgSO₄), filtered, and concentrated to give 1.05 g of a sticky solid. Purification by silica gel chromatography (6/4 hexanes/EtOAc) gave 505 mg (62%) of compound 17 as a slightly yellow oil: ¹H NMR (CDCl₃) δ 1.47 (s, 9H), 3.67-3.78 (m, 6H), 3.80 (m, 2H), 4.02 (m, 2H), 4.48 (m, 2H), 7.40 (d, 2H), 7.50 (s, 1H), 8.29 (d, 2H); mass spectrum (ES) m/z calculated for C₁₈H₂₆N₂O₁₀Na (M+Na): 453.1. Found: 453.0.

Boc-protected AOTEG/PIZ/DEA/DEG platform, compound 19: To a solution of compound 18 (prepared as described in U.S. Serial No. 60/111,641, filed Dec. 9, 1998) in a mixture of aqueous sodium bicarbonate and dioxane is added a solution of four equivalents of compound 17 in dioxane. Upon completion of the reaction, the mixture is partitioned between water and CH₂Cl₂. The CH₂Cl₂ layer is concentrated, dried, and purified by silica gel chromatography to provide compound 19.

AOTEG/PIZ/DEA/DEG platform, compound 20: The Boc-protecting groups are removed from compound 19 in a manner essentially similar to that described for the preparation of compound 16 to provide compound 20.

### Example 5A

### Synthesis of AOTEG/SA/AHAB/TEG Platform

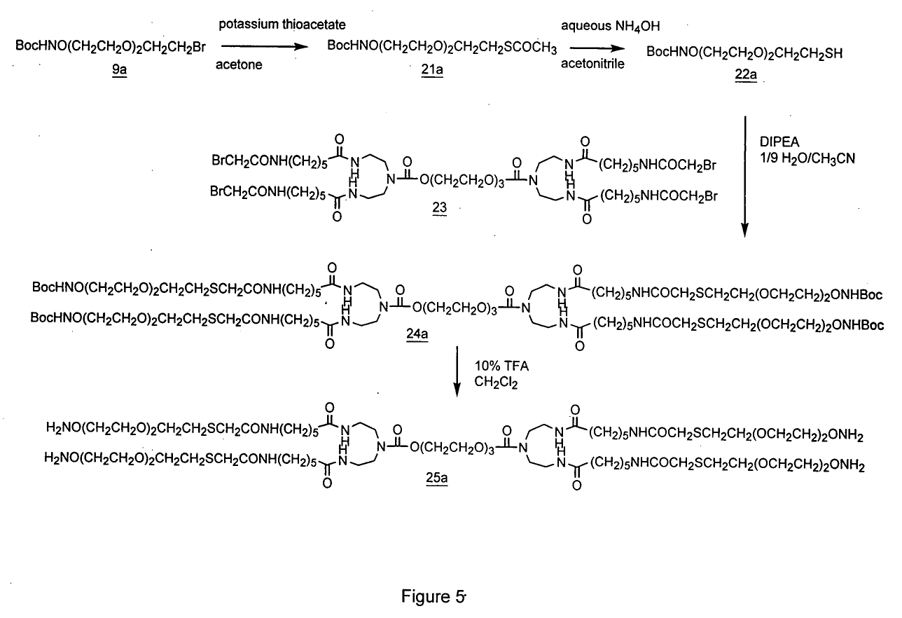

The synthetic scheme is shown in FIG. 5.

S-acetyl-2-[2-(2-N-tert-butyloxycarbonylaminooxyethyoxy)ethoxy]-ethylmercaptan, Compound 21 a: To a solution of 500 mg (1.52 mmol) of compound 9a in 30 mL of acetone was added 191 mg (1.68 mmol) of potassium thioacetate (Aldrich Chemical Co.). The mixture was stirred at room temperature for 18 hours, and the resulting precipitate was removed by filtration. The filtrate was concentrated and partitioned between 300 mL of EtOAc and 2×80 mL of brine. The EtOAc layer was dried (NaSO₄), filtered, and concentrated to give 460 mg (93%) of compound 21a as a light brown oil: ¹H NMR (CDCl₃) δ 1.48 (s, 9H), 2.35 (s, 3H), 3.12 (t, 2H), 3.61 (t, 2H), 3.64 (m, 4H), 3.73 (m, 2H), 4.02 (m, 2H), 5.52 (s, 1H); ¹³C NMR (CDCl₃) δ 28.3, 28.8, 30.6, 69.3, 69.8, 70.2, 70.5, 75.3, 81.5, 156.8, 195.3.

2-[2-(2-N-tert-butyloxycarbonylaminooxyethyoxy)ethoxy]ethylmercaptan, Compound 22a: Compound 21a is treated with a nitrogen sparged solution of 4/1 6N NH₄OH/CH₃CN in a nitrogen atmosphere for 1 hour at room temperature. The mixture is concentrated under vacuum to provide compound 22a which can be used without further purification.

Boc-Protected AOTEG/SA/AHAB/TEG platform, 24a: Compound 23 (prepared as described; Jones et al. J. Med. Chem. 1995, 38, 2138-2144.) is added to a solution of four equivalents of compound 22a in nitrogen sparged 10/90H₂O/CH₃CN. To the resulting solution is added four equivalents of diisopropylethylamine. Upon completion of the reaction, the mixture is partitioned between water and CH₂Cl₂. The CH₂Cl₂ layer is concentrated, dried, and purified by silica gel chromatography to provide compound 24a.

AOTEG/SA/AHAB/TEG platform, 25a: The Boc-protecting groups are removed from compound 24a in a manner essentially similar to that described for the preparation of compound 16 to provide compound 25a.

### Example 5B

### Synthesis of AOHEX/SA/AHAB/TEG Platform

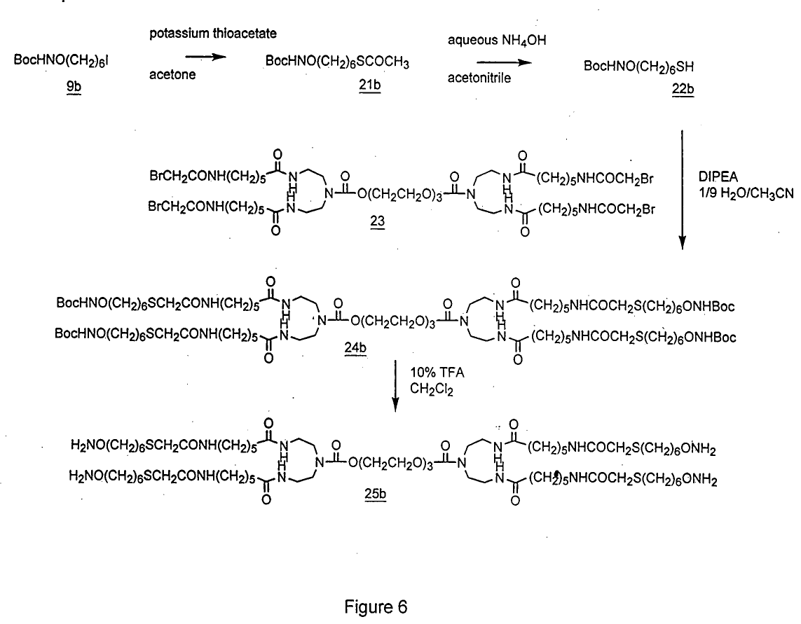

The synthetic scheme is shown in FIG. 6.

1-Iodo-6-(N-tert-butyloxycarbonyl)aminooxyhexane, compound 9b: To a heterogeneous mixture of 140 mg (1.05 mmol) of N-(tert-butyloxycarbonyl)hydroxylamine (Aldrich Chemical Co.) and 658 μL (1.35 mg, 4.0 mmol) of compound 12 was added 149 μL (152 mg, 1.0 mmol) of DBU. The mixture was stirred at room temperature for 30 seconds at which time the reaction mixture solidified. The solid mass was allowed to stand overnight and was dissolved in 50 mL of CH₂Cl₂. The solution was washed with 2×25 mL of 1 N NaOH and 3×25 mL of 1 N HCl. The combined basic aqueous layers were extracted with 25 mL of CH₂Cl₂, and the combined acidic aqueous layers were extracted with 25 mL of CH₂Cl₂. The combined CH₂Cl₂ layers were dried (Na₂SO₄), filtered, and concentrated to give a yellow oil. Purification by silica gel chromatography (step gradient; 1/99/0.1 to 15/85/0.1 EtOAc/hexanes/MeOH) provided 216 mg (68%) of 9b as a yellow oil: ¹H NMR (CDCl₃) δ 1.40 (m, 4H), 1.48 (s, 9H), 1.62 (m, 2H), 1.83 (m, 2H), 3.20 (t, 2H), 3.84 (t, 2H), 7.10 (s, 1H).

S-acetyl-6-(N-tert-butyloxycarbonyl)aminooxyhexan-1-thiol, Compound 21 b: Compound 9b (209 mg, 0.61 mmol) was added to a solution of potassium thioacetate in 15 mL of acetone, and the mixture was stirred at room temperature for 18 hours. The acetone was removed under vacuum, and the residue was partitioned between 50 mL of CH₂Cl₂ and 3×25 mL of 1 N NaOH. The CH₂Cl₂ layer was dried (Na₂SO₄), filtered, and concentrated to give a brown oil. Purification by silica gel chromatography (15/85 EtOAc/hexanes) provided 166 mg (94%) of compound 21b as a colorless oil: ¹H NMR (CDCl₃) δ 1.39 (m, 4H), 1.48 (s, 9H), 1.59 (m, 4H), 2.32 (s, 3H). 2.86 (t, 2H), 3.82 (t, 2H), 7.10 (s, 1H).

6-(N-tert-butyloxycarbonyl)aminooxyhexan-1-thiol, Compound 22b: A purified sample of 22b was prepared as follows. Compound 21b (50 mg, 172 μmol) and 22 μL (17.4 mg, 85.8 μmol) of tri-n-butylphosphine was placed under nitrogen, and 2 mL of a nitrogen sparged 1 M solution of NaOH in MeOH was added to the mixture. The mixture was stirred for 18 hours at room temperature, and 172 μL (180 mg, 3 mmol) of trifluoroacetic acid was added. The mixture was partitioned between 25 mL of EtOAc and 3×25 mL of 1 N HCl. The combined aqueous layers were extracted with 25 mL of EtOAc, dried (Na₂SO₄), filtered, and concentrated to give an oil. Purification by silica gel chromatography (15/85/0.1 EtOAc/hexanes/MeOH) provided 28 mg of 22b as a colorless oil: ¹H NMR (CDCl₃) δ 1.32 (t, 1H), 1.40 (m, 4H), 1.49 (s, 9H), 1.62 (m, 4H), 2.53 (d oft, 2H). 3.84 (t, 2H), 7.09 (s, 1H).

Boc-Protected AOHEX/SA/AHAB/TEG platform, 24b: Compound 21b (13 mg, 45 μmol) and 6 μL (4.5 mg, 22.3 μmol) of tri-n-butylphosphine was placed under nitrogen, and 3 mL of a nitrogen sparged solution of 4/1 6 N NH₄OH/CH₃CN was added to the mixture. The mixture was stirred for 1 hour at room temperature and concentrated under vacuum. The residue was dissolved in 3 mL of a nitrogen sparged solution of 10/90 water/CH₃CN. To the resulting solution, which was kept under nitrogen atmosphere, was added 10 mg (7.44 μmol) of compound 23 followed by 8 μL (5.77 mg, 44.6 μmol) of diisopropylethylamine. The mixture was stirred for 18 hours and concentrated under vacuum. The residue was purified by silica gel chromatography (multiple step gradient, 1/99 to 5/95 to 7.5/92.5 to 10/90 to 15/85 MeOH/CH₂Cl₂) to provide 14 mg (93%) of 24b as a colorless oil: TLC (10/90 MeOH/CH₂Cl₂), R_{f}=0.3; mass spectrum (ES) m/z calculated for C₉₂H₁₇₄N₁₄O₂₆S₄ (M+2H)/2: 1010. Found: 1010.

AOHEX/SA/AHAB/TEG platform, 25b: The Boc-protecting groups are removed from compound 24b in a manner essentially similar to that described for the preparation of compound 16.

### Example 6

### Synthesis of AOHOC/DT/TEG Platform

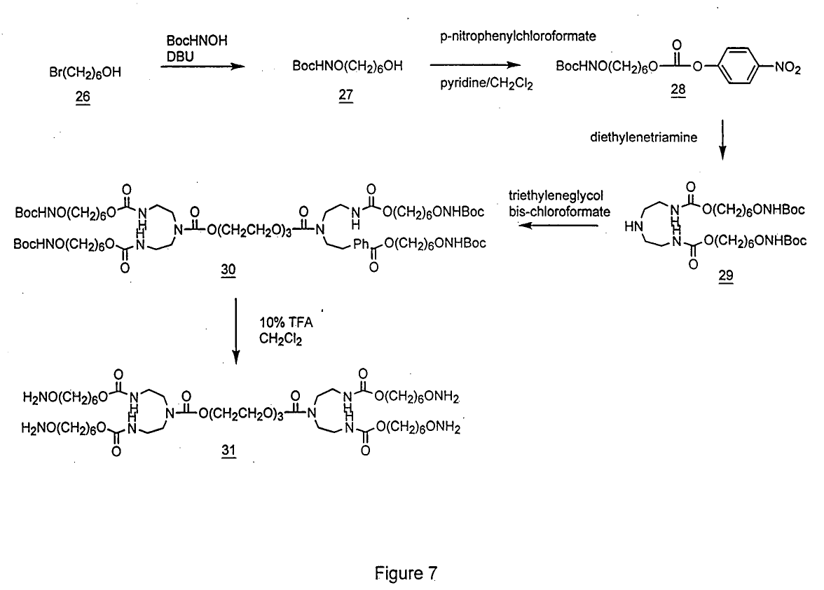

The synthetic scheme is shown in FIG. 7.

6-(tert-butyloxycarbonylaminooxy)hexan-1-ol, 27: To a solution of 179 μL (183 mg, 1.2 mmol) of DBU in 1 mL of CH₂Cl₂ was added 133 mg (1.0 mmol) of N-(tert-butyloxycarbonyl)hydroxylamine (Aldrich Chemical Co.) and 157 μL (217 mg, 1.2 mmol) of 6-bromohexan-1-ol (Aldrich Chemical Co.), and the mixture was stirred for 18 hours at room temperature. The mixture was concentrated to give a yellow oil. Purification by silica gel chromatography (35/5/65 EtOAc/MeOH/hexanes) gave 180 mg (77%) of compound 27 as a colorless oil: ¹H NMR (CDCl₃) δ 1.39 (m, 4H), 1.48 (s, 9H), 1.59 (m, 4H), 3.63 (t, 2H), 3.85 (t, 2H), 7.42 (s, 1H); ³C NMR (CDCl₃) δ 25.6, 25.8, 28.1, 28.4, 62.8, 76.8, 81.7, 157.2.

Compound 28: To a solution of 100 mg (0.428 mmol) of compound 27 in 2 mL of CH₂Cl₂ at 0° C. was added 90 μL (88.1 mg, 1.1 μmmol) of pyridine followed by 113 mg (0.557 mg) of p-nitrophenylchloroformate (Aldrich Chemical Co.). The mixture was stirred at room temperature for 4 hours, cooled to 0° C., acidified with 1 N HCl, and partitioned between 20 mL of 1 N HCl and 3×20 mL of CH₂Cl₂. The combined CH₂Cl₂ layers were washed with a saturated solution of NaHCO₃, dried (MgSO₄), filtered, and concentrated. Purification by silica gel chromatography to provided compound 28.

Compound 29: To a solution of diethylenetriamine in EtOAc is added two equivalents of diisopropylethylamine followed by two equivalents of compound 28. The mixture is stirred until the reaction is complete. The solvents are removed and the product, compound 29, is purified by silica gel chromatography.

Boc-protected AOHOC/DT/TEG Platform, 30: To a solution of triethylene glycol bis-chloroformate (Aldrich Chemical Co.) in pyridine is added two equivalents of compound 29. The mixture is stirred until the reaction is complete and partitioned between 1 N HCl and CH₂Cl₂. The CH₂Cl₂ layer is dried and concentrated, and the product is purified by silica gel chromatography to give compound 30.

AOHOC/DT/TEG Platform, 31: The Boc-protecting groups are removed from compound 30 in a manner essentially similar to that described for the preparation of compound 16.

### Example 7

### Synthesis of AOTEG/IDA/TEG Platform

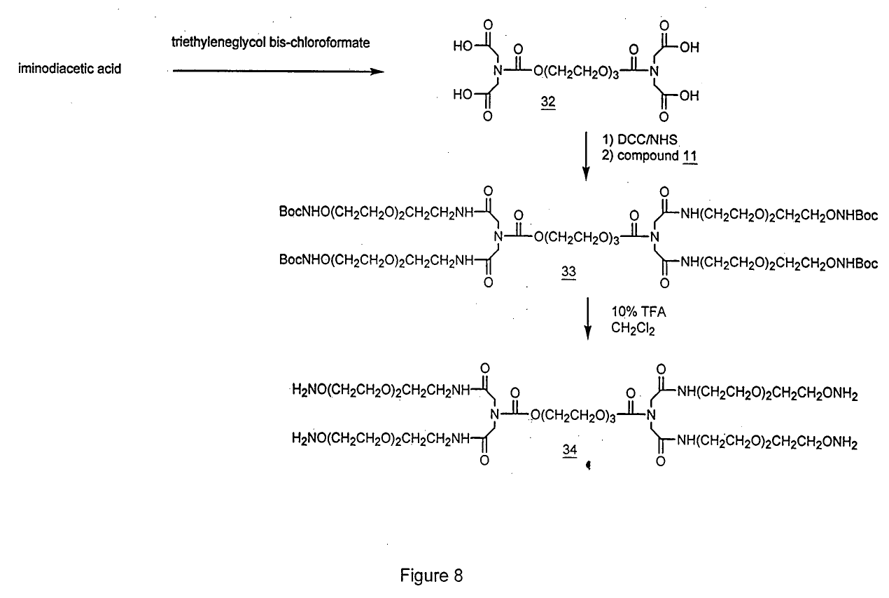

The synthetic scheme is shown in FIG. 8.

Compound 32: To a solution of triethylene glycol bis-chloroformate (Aldrich Chemical Co.) in pyridine is added two equivalents of iminodiacetic acid (Aldrich Chemical Co.). The mixture is stirred until the reaction is complete and partitioned between 1 N HCl and CH₂Cl₂. The CH₂Cl₂ layer is dried and concentrated, and the product is purified by silica gel chromatography to give compound 32.

Compound 33: A solution of compound 32 in THF is treated with 6 equivalents of NHS and 6 equivalents of DCC for 1 hour. To the mixture is added 4 equivalents of compound 11, and the mixture is stirred until the reaction is complete. Acetic acid is added to quench excess DCC, and the resulting solids are removed by filtration. The filtrate is concentrated and purified by silica gel chromatography to provid compound 33.

Compound 34: The Boc-protecting groups are removed from compound 33 in a manner essentially similar to that described for the preparation of compound 16.

### Example 8

### Synthesis of AOTEGO/LEV/PITG Platform

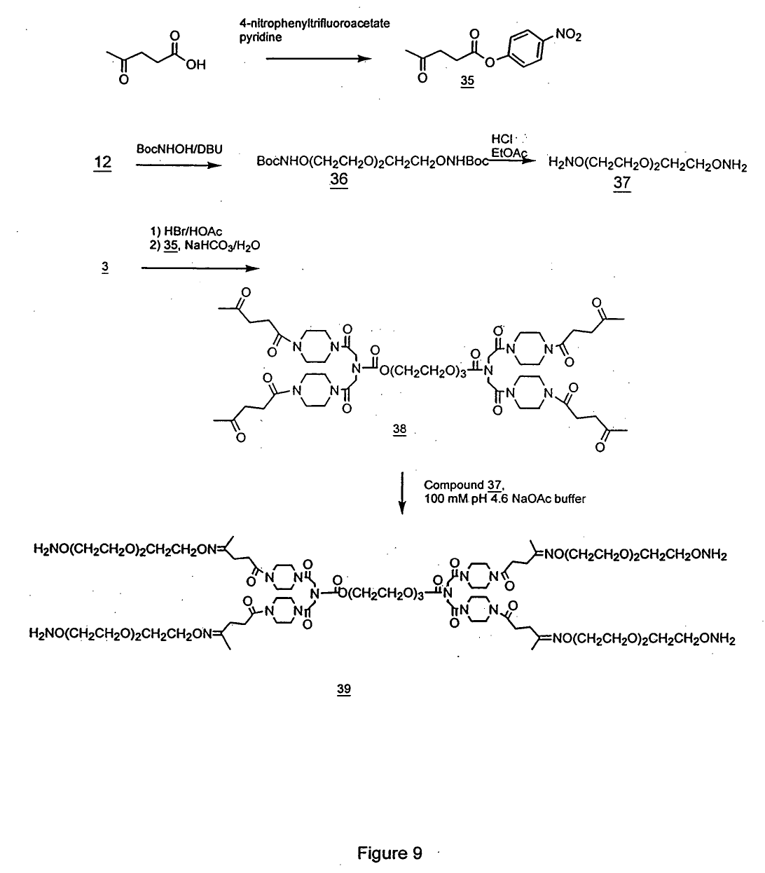

The synthetic scheme is shown in FIG. 9.

p-Nitrophenyl-levulinate, 35: To a solution of 800 mg (6.89 mmol) of levulinic acid (Aldrich Chemical Co.) in 4.25 mL of pyridine was added 1.78 g (7.58 mmol) of 4-nitrophenyltrifluoroacetate (Aldrich Chemical Co.). The resulting solution was stirred for 15 minutes and partitioned between 28 mL of water and 2×28 mL of CH₂Cl₂. The combined CH₂Cl₂ layers were dried (MgSO₄), filtered, and concentrated. Purification of the concentrate by silica gel chromatography (step gradient, 25/75 to 30/70 EtOAc/hexanes) provided 1.06 g (74%) of compound 35: ¹H NMR (CDCl₃) δ 2.28 (s, 3H), 2.87 (m, 4H), 7.29 (d, 2H), 8.28 (d, 2H).

1,2-Bis(2-(tert-butyloxycarbonyl)aminooxyethoxy)ethane, compound 36: To 243 mg (0.66 mmol) of compound 12 was added 219 mg (1.64 mmol) of N-(tert-butyloxycarbonyl)hydroxylamine (Aldrich Chemical Co.) followed by 246 μL (250 mg, 1.64 mmol) of DBU. The mixture was stirred at room temperature until it solidified (approximately 1 hour). After standing for an additional hour, the mixture was dissolved in 2 mL of CH₂Cl₂, and the resulting solution was added to 100 mL of EtOAc to precipitate the hydrogen-iodide salt of DBU. An additional 50 mL of EtOAc was added, and the mixture was filtered. The filtrate was washed with 2×50 mL of 1 N HCl, 2×50 mL of 5% sodium bisulfite solution, and 25 mL of brine. The EtOAc layer was dried (Na₂SO₄), filtered, and concentrated to give an oil. Purification by silica gel chromatography (step gradient, 40/60 to 50/50 to 80/20 EtOAc/hexanes) to give 164 mg (65%) of compound 36 as a colorless oil: ¹H NMR (CDCl₃) δ 1.48 (s, 18H), 3.65 (s, 4H), 3.72 (t, 4H), 4.02 (t, 4H), 7.80 (s, 2H); ¹³C NMR (CDCl₃) δ 28.2, 69.0, 70.3, 75.2, 81.3, 156.8.

1,2-Bis(2-aminooxyethoxy)ethane, compound 37: Compound 36 (559 mg, 1.47 mmol) was dissolved in 15 mL of of EtOAc, and HCl gas was bubbled through the solution for 30 minutes. The mixture was concentrated under vacuum to provide 72 mg (90%) of compound 37 as the HCl salt as a sticky residue: ¹H NMR (D₂O) δ 3.75 (s, 4H), 3.87 (m, 4H), 4.27 (m, 4H); mass spectrum (ES) m/z calculated for C₆H₁₇N₂O₄ (M+H): 181.1. Found: 181.1.

Compound 38: Compound 3 is treated with a 30% solution of HBr in acetic acid to remove the CBZ protecting groups and provide a tetra-amine hydrogen bromide salt. The tetra-amine is dissolved in a solution of sodium bicarbonate in water and dioxane, and to the resulting solution is added four equivalents of compound 35. Upon completion of the reaction, the mixture is partitioned between water and CH₂Cl₂. The CH₂Cl₂ layer is concentrated, dried, and purified by silica gel chromatography to provide compound 38.

AOTEGO/LEV/PITG Platform, compound 39: To a solution of compound 38 in 0.1 M pH 4.6 sodium acetate buffer is added twenty equivalents of compound 37. Upon completion of the reaction, the mixture is partitioned between water and CH₂Cl₂. The CH₂Cl₂ layer is concentrated, dried, and purified by silica gel chromatography to provide compound 39.

### Example 9

### Synthesis of AO/DEGA/DEG Platform

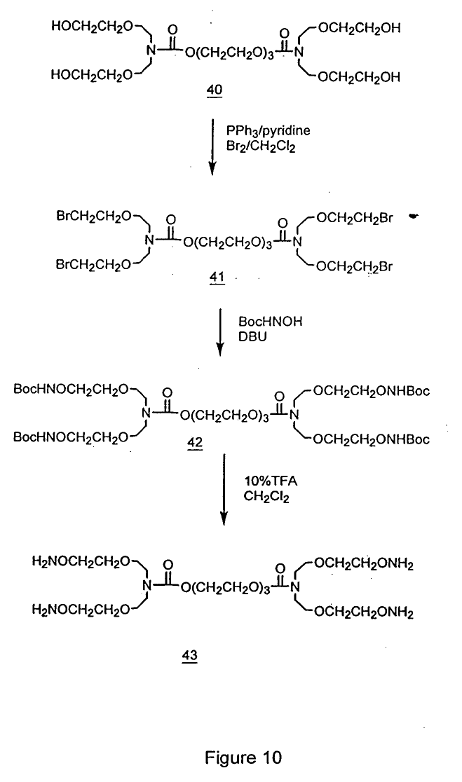

The synthetic scheme is shown in FIG. 10.

Compound 41: Bromine (approximately six equivalents) is added dropwise to a solution of compound 40, six equivalents of triphenylphosphine, and 8 equivalents of pyridine in CH₂Cl₂ until an orange color persists. The mixture is stirred at room temperature for 0.5 h or until reaction is complete, and a saturated solution of sodium bisulfite is added to destroy excess bromine. The mixture is then partitioned between H₂O and EtOAc. The combined organic layers are washed with brine, dried (Na₂SO₄), filtered, concentrated, and purified by silica gel chromatography to provide compound 41.

Compound 42: To compound 41, is added six equivalents of N-(tert-butyloxycarbonyl)hydroxylamine (Aldrich Chemical Co.) and six equivalents of DBU. The mixture is heated as necessary for a sufficient time for the reaction to come to completion. When cool, the mixture is dissolved in CH₂Cl₂ and the resuting solution is added to EtOAc resulting in the formation of a precipitate which is removed by filtration, and the filtrate is concentrated. Purification by flash chromatography provides 42.

Compound 43: The Boc-protecting groups are removed from compound 42 in a manner essentially similar to that described for the preparation of compound 16.

### Example 10

### Synthesis of Tetravalent D1 Conjugate

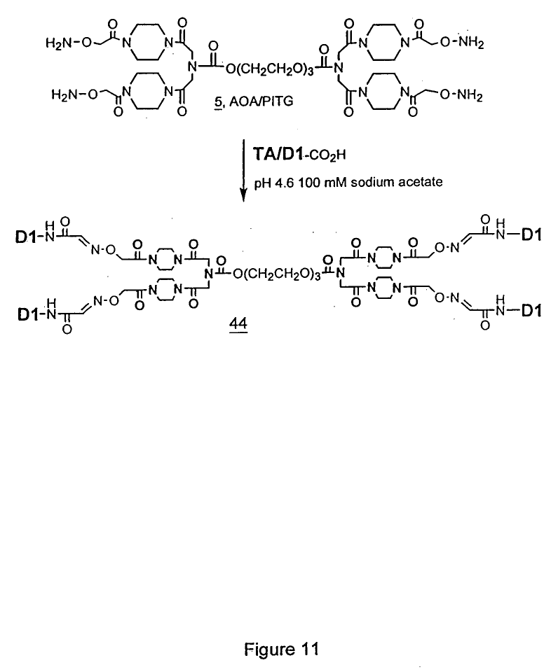

The synthetic scheme is shown in FIG. 11.

Synthesis of Tetravalent D1 Conjugate, Compound 44: TA/D1, prepared as described in Example 1 (0.90 mg, 1.28×10⁻⁷ mol) was dissolved in 250 μL of 0.1 M sodium acetate pH 4.60 buffer in a polypropylene tube. To the mixture was added 16.6 μL (18.9 ug, 1.60×10⁻⁸ mol) of a 0.97 μmol/mL solution of AOA/PITG platform, compound 5, in 0.1 M sodium acetate pH 4.60 buffer. The mixture was agitated gently under nitrogen for 6 days at which time the reaction appeared to be complete by analytical HPLC using a 4.6 mm×250 mm, 300 Å, 5 μm, diphenyl column (Vydac) with detection at 280 nm (1 Mt/min; gradient 25%-45% B, 0-20 min, A=0.1% TFA/H₂O, B=0.1% TFA/CH₃CN). Approximate retention times are as follows: TA/D1, 13.7 min; compound 44, 17.2 min). The mixture was diluted with 95/5 water/acetonitrile to a volume of 1 mL and purified by HPLC (10 mm×250 mm, 300 Å, 5 μm, diphenyl column (Vydac) (3 mL/min; gradient 25%-45% B, 0-40 min, A=0.1% TFA/H₂O, B=0.1% TFA/CH₃CN). Fractions containing pure 44, as evidenced by analytical HPLC, were pooled and lyophilized to provide 0.4 mg (25%) of 44: mass spectrum (ES, average m/z) caculated for C₁₃₂₀H₂₀₃₂N₃₃₈O₃₇₀S₂₀: 29,198. Found: 29,218.

### Example 11

### Synthesis of Tetravalent D1 Conjugate

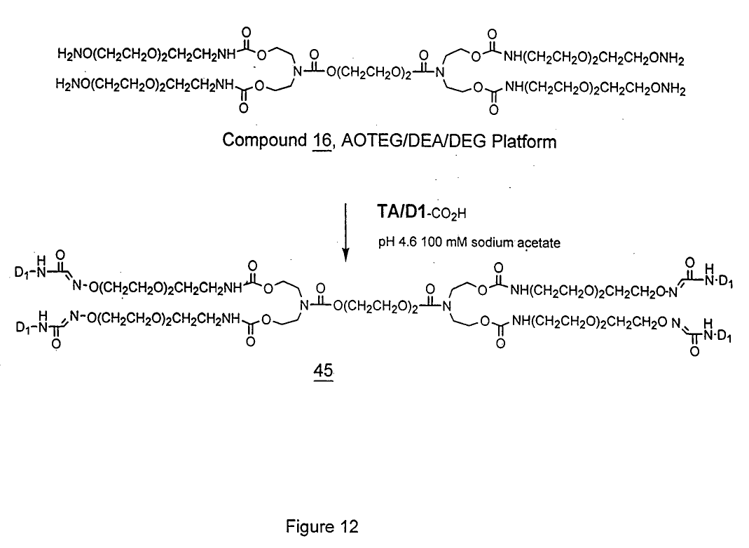

The synthetic scheme is shown in FIG. 12.

Synthesis of Tetravalent D1 Conjugate, Compound 45: TA/D1, prepared as described in Example 1, (5.20 mg, 7.37×10⁻⁷ mol) was dissolved in 2.0 mL of He sparged 0.1 M sodium acetate pH 4.60 buffer in a polypropylene tube. To the mixture was added 15.07 μL (139 ug, 1.23×10⁻⁷ mol) of a 8.147 μmol/mL solution of AOTEG/DEA/DEG platform, compound 16, in 0.1 M sodium acetate pH 4.60 buffer. The mixture was agitated gently under nitrogen for 23 hours at which time the reaction appeared to be complete by analytical HPLC using a 4.6 mm×250 mm, 300 Å, 5 μm, diphenyl column (Vydac) with detection at 280 nm (1 mL/min; gradient 25%-45% B, 0-20 min, A=0.1% TFA/H₂O, B=0.1% TFA/CH₃CN). Approximate retention times are as follows: TA/D1, 13.7 min; 45, 17.2 min). The mixture was diluted with water to a volume of 5 mL and purified by HPLC (10 mm×250 mm, 300 Å, 5 μm, diphenyl column (Vydac) (3 mL/min; gradient 25%-45% B, 0-40 min, A=0.1% TFA/H₂O, B=0.1% TFA/CH₃CN). Fractions containing pure 45, as evidenced by analytical HPLC, were pooled and lyophilized to provide 1.73 mg (48%) of 45: mass spectrum (ES, average m/z) caculated for C₁₃₂₂H₂₀₄₈N₃₃₄O₃₇₇S₂₀: 29,294. Found: 29,294.

### Example 12

### Preparation of Model Aminooxy Compounds and Comparison of Reactivities with Glyoxyl-Peptide

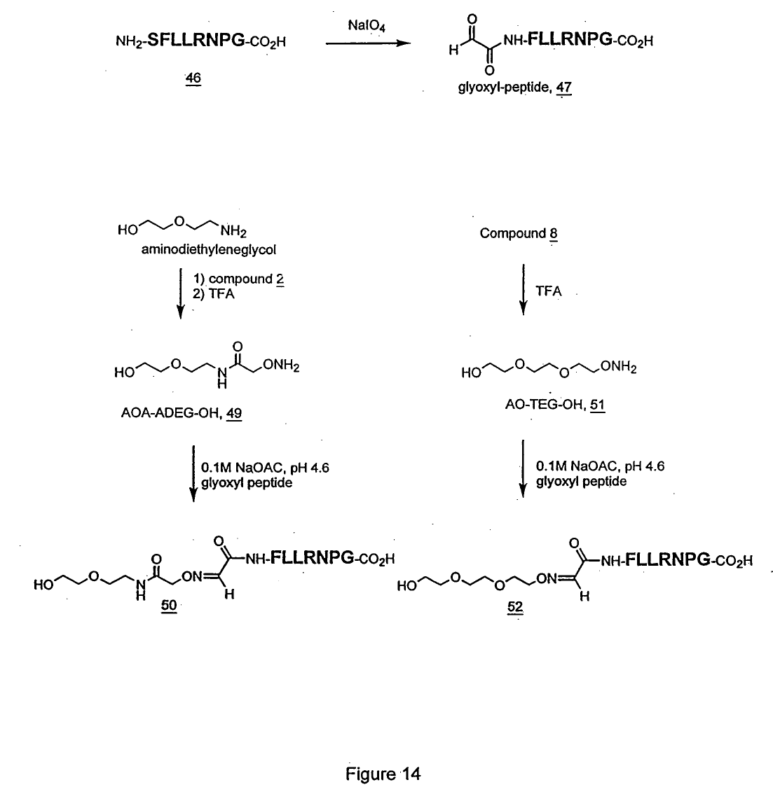

The synthetic scheme is shown in FIG. 14.

Synthesis of glyoxyl-peptide, compound 47: Compound 46 (SEQ. ID No. 1) was prepared by standard solid phase synthesis on Wang resin, using N-Fmoc protected aminoacids. Couplings were done with 3 equivalents of N-Fmoc-protected aminoacid, 3 equivalents of DIC, and 3 equivalents of HOBt in DMF. Deprotections were done with 20% pyridine in DMF. The peptide was cleaved from the resin and purified by reversed phase HPLC (C₁₈, gradient, 10-30% B, 0-40 min, A=0.1% TFA/H₂O, B=0.1% TFA/CH₃CN). The pure fractions, as evidenced by analytical HPLC (4.6×250 mm C₁₈, 1 mL/min, gradient, 10-60% B, 0-20 min, A=0.1% TFA/H₂O, B=0.1% TFA/CH₃CN, Tᵣ=10.3 min) were lyophilized to provide compound 46 as a fluffy white solid: mass spectrum (ES) calculated for (M+H) C₄₁H₆₇N₁₂O₁₁: 903.5. Found: 903.5.

To a solution of 163 mg (0.18 mmol) of compound 46 in 3.67 mL of CH₃CN and 19 mL of 10 mM sodium phosphate pH 7.0 buffer was added a solution of 77.2 mg (0.361 mmol) of sodium periodate in 5.4 mL of water. The mixture was stirred at room temperature for 30 minutes, and 100 μL of acetic acid was added. The mixture was filtered, and the filtrate was purified by HPLC (C₁₈, gradient, 15-30% B, 0-40 min, A=0.1% TFA/H₂O, B=0.1% TFA/CH₃CN). The pure fractions, as evidenced by analytical HPLC (4.6×250 mm C₁₈, 1 mL/min, gradient, 10-35% B, 0-20 min, A=0.1% TFA/H₂O, B=0.1% TFA/CH₃CN, Tᵣ=17.6 min), were lyophilized to provide to give 124 mg (79%) of compound 47 (SEQ ID No. 2) as a white solid after lyophilization: mass spectrum (ES) calculated for (M+H)C₄₀H₆₂N₁₁O₁₁: 872.5. Found: 872.5.

Synthesis of compound 49: To a solution of 250 mg (0.801 mmol) of compound 2 in 5 mL of CH₂Cl₂ was added 158 μL (166 mg, 1.58 mmol) of aminodiethyleneglycol (Aldrich Chemical Co.). To the resulting solution was added 298 μL (221 mg, 1.71 mmol) of diisopropylethylamine, and the mixture was stirred under nitrogen atmosphere at room temperature for 1.5 hours. The mixture was partitioned between 100 mL of CH₂Cl₂ and 20 mL of saturated Na₂CO₃ solution, the CH₂Cl₂ layer was washed successively with two 20 mL portions of saturated Na₂CO₃ solution, two 20 mL portions of 1 N HCl, and 20 mL of brine. The aqueous HCl layer was extracted with five 50 mL portions of CH₂Cl₂; the aqueous Na₂CO₃ layer was extracted with two 50 mL portions of CH₂Cl₂. The combined organic layers were dried (MgSO₄), filtered, and concentrated to give a yellow oil. Purification by silica gel chromatography (70/30 EtOAc/hexanes) gave 164 mg (73%) of the Boc-protected precurser to compound 49 as a sticky colorless oil: ¹H NMR (CDCl₃) δ 1.48 (s, 9H), 3.52 (m, 2H), 3.62 (m, 4H), 3.77 (m, 2H), 4.35 (s, 2H), 7.64 (s, 1H), 8.33 (brd s, 1H).

The Boc protecting group was removed as follows. The Boc protected precurser (164 mg, 0.59 mmol) was dissolved in 5 mL of 50/50 trifluoroacetic acid/CH₂Cl₂ and the mixture was stirred for two hours at room temperature. The mixture was evaporated under a gentle stream of nitrogen, and the residue was redissolved in CH₂Cl₂. The solution was concentrated under vacuum to give 179 mg (104% of theory, remainder assumed to be TFA) of the trifluoroacetate salt of compound 49 as a colorless oil: mass spectrum (ES) calculated for (M+H)C₆H₁₅N₂O₄: 179.2. Found: 179.1.

Synthesis of compound 50: To a solution of 5.0 mg (5.62 μmol) of compound 47 in 7.6 mL of 0.1 M pH 4.6 sodium acetate buffer was added 582 μL of a solution of 3.29 mg of compound 49 (estimated purity 96%, 1.70 mg, 5.82 [mol) in 10 mL of 0.1 M pH 4.6 sodium acetate buffer, and the mixture was stirred for six days. The mixture was purified directly by HPLC (C₁₈; gradient, 25%-45% B, 0-40 min, A=aqueous pH 7.0 triethylammonium phosphate (prepared by mixing 500 mL of 0.1% H₃PO₄ with approximately 500 mL of 0.3% Et₃N to provide a pH of 7.0), B=CH₃CN). Fractions containing product were lyophilized to provide 0.3 mg of compound 50: mass spectrum (ES) calculated for (M+H)C₄₆H₇₄N₁₃O₁₄: 1032.5. Found: 1032.6.

Synthesis of compound 51: Compound 8 (100 mg, 0.38 mmol) was dissolved in 25 mL of 1/9 trifluoroacetic acid/CH₂Cl₂ and the mixture was allowed to stand for 2 hours at room temperature. The mixture was evaporated under a gentle stream of nitrogen, and the residue was redissolved in CH₂Cl₂. The solution was concentrated under vacuum to give 152 mg (145% of theory, remainder assumed to be TFA) of the trifluoroacetate salt of compound 51 as a colorless oil: mass spectrum (ES) calculated for (M+H)C₆H₁₆NO₄: 165.1. Found: 165.1.

Synthesis of compound 52: To a solution of 5.0 mg (5.62 μmol) of compound 47 in 7.6 mL of 0.1 M pH 4.6 sodium acetate buffer was added 845 μL of a solution of 3.29 mg of compound 51 (estimated purity 69%, 1.63 mg, 5.82 μmol) in 10 mL of 0.1 M pH 4.6 sodium acetate buffer, and the mixture was sirred for 21 hours. The mixture was purified directly by HPLC (C₁₈; gradient, 25%-45% B, 0-40 min, A=aqueous pH 7.0 triethylammonium phosphate (prepared by mixing 500 mL of 0.1% H₃PO₄ with approximately 500 mL of 0.3% Et₃N to provide a pH of 7.0), B CH₃CN). Fractions containing product were lyophilized to provide 3 mg of compound 52: mass spectrum (ES) calculated for (M+H)C₄₆H₇₅N₁₂O₁₄: 1019.5. Found: 1019.5.

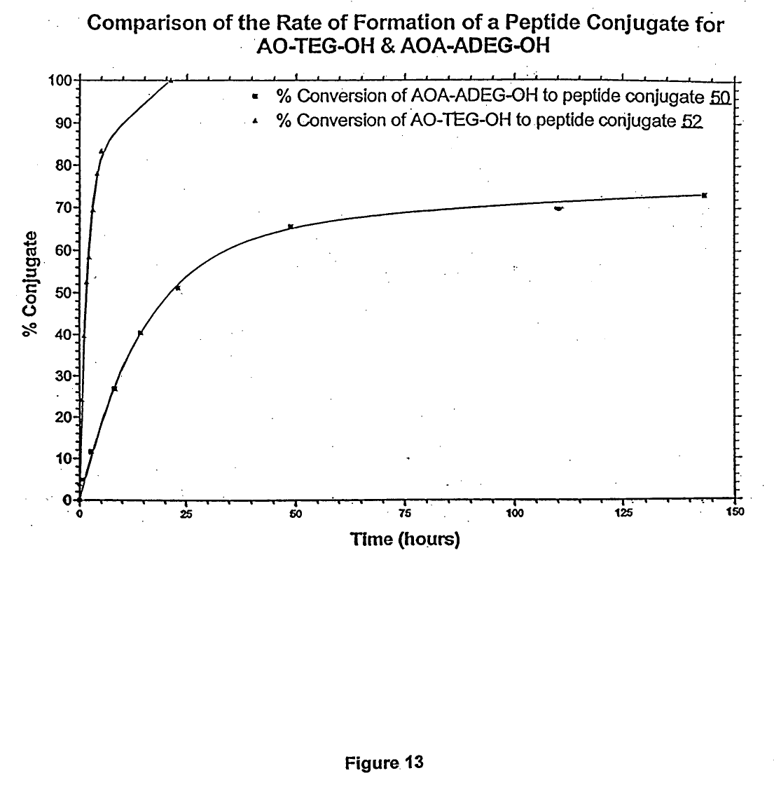

Comparison of rates of conversion of 49 to 50 and 51 to 52: The rates of conversion of 49 (AOA-ADEG-OH, comprising an aminooxyacetyl group) to product 50, and 51 (AO-TEG-OH, comprising an aminooxyalkyl group) to 52, were measured by injecting aliquots of reaction mixture onto an analytical HPLC at various time points, and measuring the amount of product at that time by analytical HPLC (C₁₈, gradient, 10-60% B, 0-40 min, A=0.1% TFA/H₂O, B=0.1% TFA/CH₃CN). As illustrated in FIG. 13, the valency platform molecule comprising aminooxyalkyl groups formed the oxime conjugate with the model peptide more quickly.

### Example 13

### Alternative Method of Preparing a Tetravalent Conjugate Using Compound 37 as a Bifunctional Linker

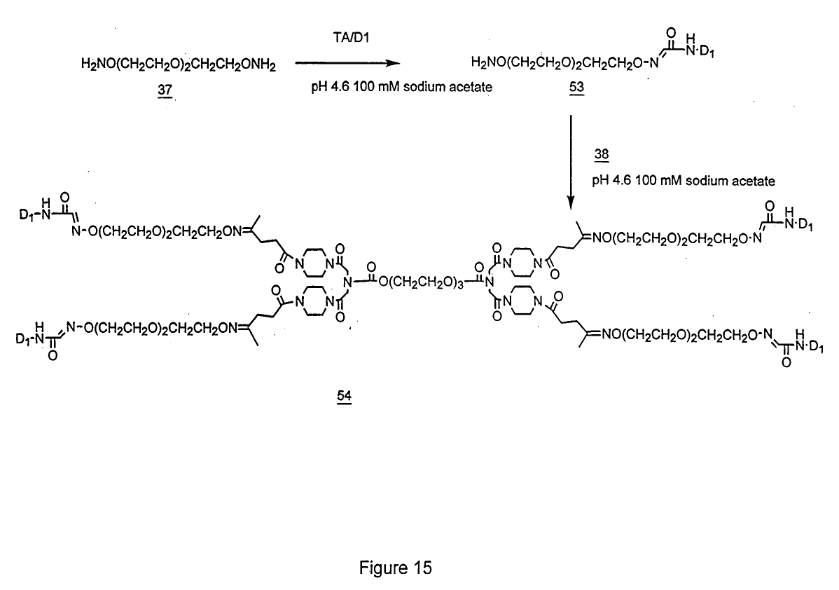

As an alternative to reacting a transaminated domain 1 β₂GPI polypeptide, or any other glyoxylated polypeptide, directly with a tetravalent aminooxy platform, a transaminated polypeptide can be reacted with an excess of compound 37 in pH 4.6 100 mM sodium acetate buffer to provide compound 53 in which an aminooxy linker is attached to the polypeptide (here, a domain 1 polypeptide) via an oxime bond. The synthetic scheme is shown in FIG. 15. Compound 53 is separated from excess linker, and four equivalents of compound 53 is reacted with platform 38 in pH 4.6 100 mM sodium acetate buffer to form a second set of oxime bonds providing a tetravalent conjugate, compound 54.

### Example 14

### Alternative Method of Preparing a Tetravalent Conjugate Using Compound 21a as a Precurser to a Bifunctional Linker

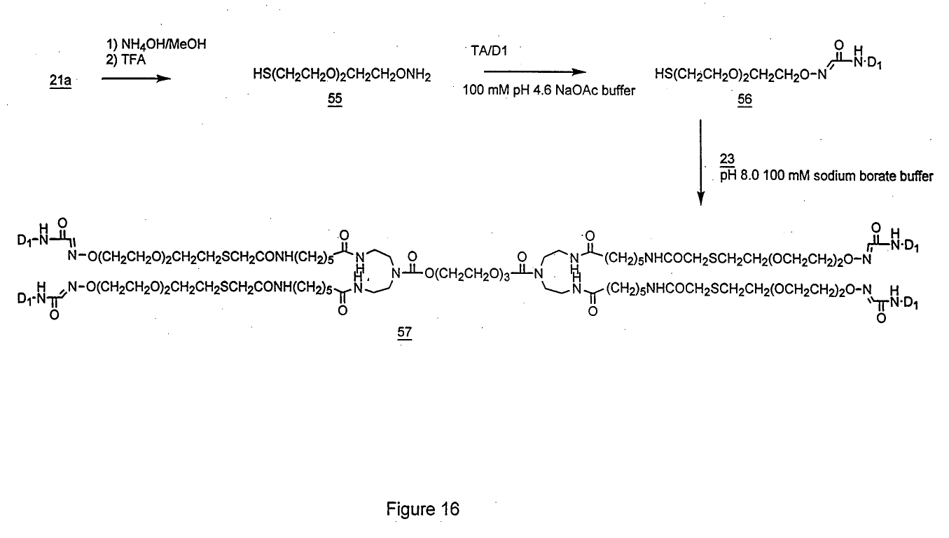

Treatment of compound 21 a with ammonium hydroxide to remove the acetyl sulfur protecting group, then with trifluoroacetic acid to remove the Boc protective group provides linker 55. A glyoxyl-containing polypeptide, in this case TA/D1, is reacted with compound 55 to provide compound 56, Domain 1 with a the sulfhydryl linker attached via an oxime bond. Four equivalents of compound 56 can react with platform 23 to provide a tetravalent domain 1 polypeptide conjugate, compound 57. The synthetic scheme is shown in FIG. 16.

### Example 15

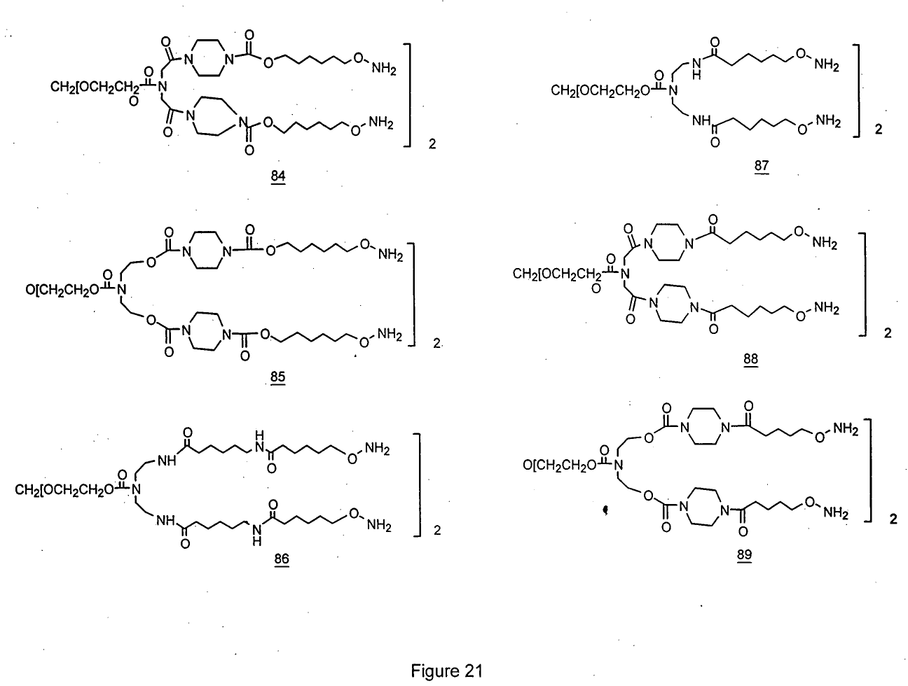

### Synthesis of compound 85, FIG. 21

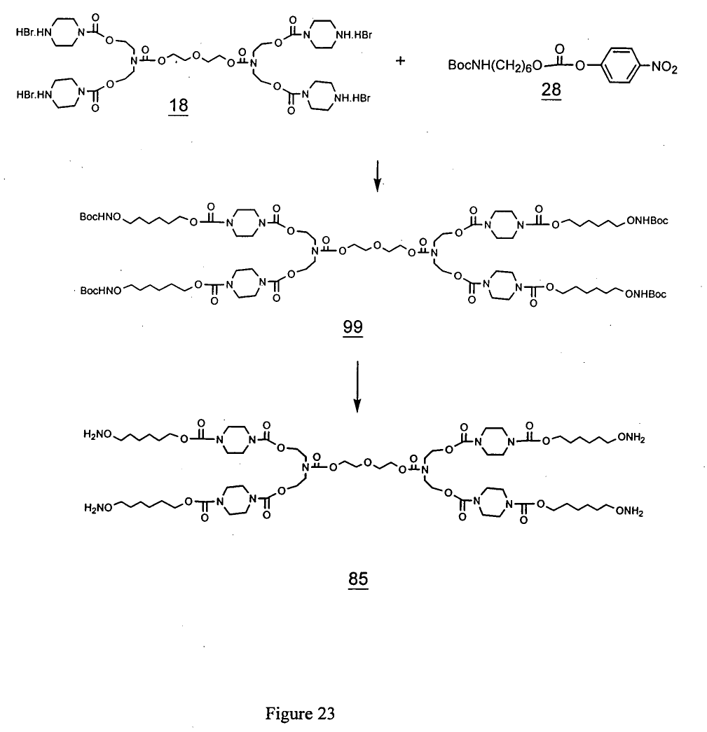

The synthesis of the aminooxy platform, compound 85, was accomplished in a manner essentially the same as the synthesis of compound 20 (shown in FIG. 4); however, compound 28 was used instead of compound 17. Compound 18 was reacted with compound 28, as shown in FIG. 23, to give the Boc-protected platform 99.

The Boc-protecting groups are removed from compound 99 in a manner essentially similar to that described for the preparation of compound 16 to provide 85.

### Example 16

### Synthesis of Compound 86, FIG. 21

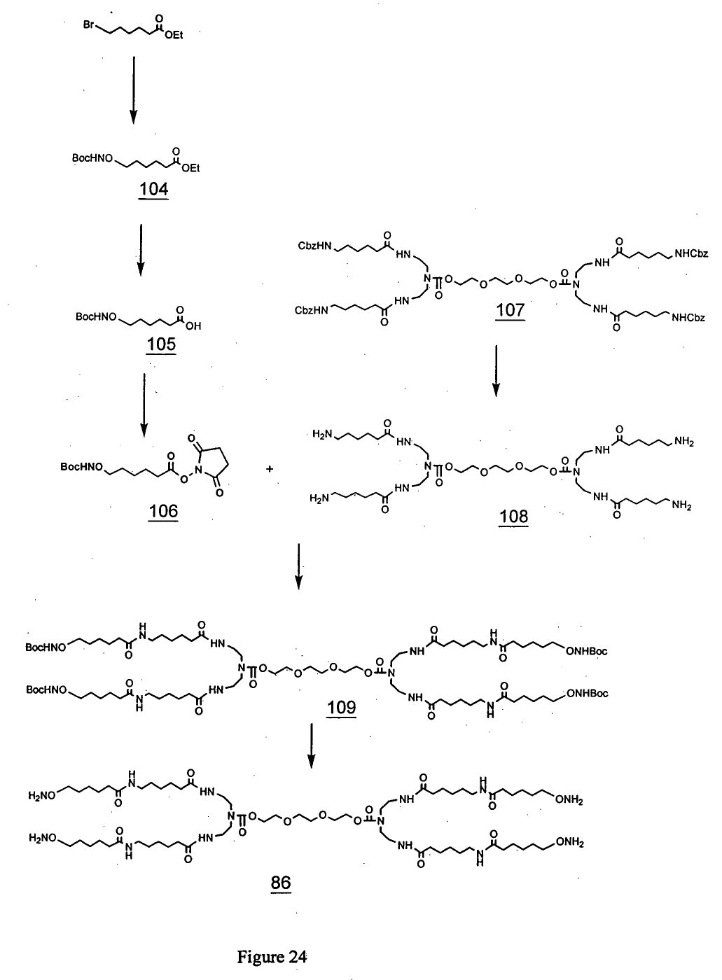

The preparation of compound 86 involved preparing Boc-protected aminooxyhexanoic acid, compound 105, and using it to acylate a tetra-amino platform, compound 108 as shown in Scheme B in FIG. 24.

Ethyl 6-(N-tert-butyloxycarbonyl)aminooxyhexanoate, compound 104:

To a magnetically-stirred mixture of 500 mg (3.76 mmol) of N-(tert-butyloxycarbonyl)hydroxylamine (Aldrich Chemical Co.) and 267 μL (335 mg, 1.50 mmol) of ethyl 6-bromohexanoate was added 1.12 mL (1.14 g, 7.51 mmol) of DBU over a period of approximately one minute. The mixture was allowed to stir for 24 hours, at which time it had partially solidified. The mixture was dissolved in 100 mL of CH₂Cl₂, and the resulting solution was shaken in a separatory funnel with four 25 mL portions of 1 N HCl and 25 mL of brine. The aqueous layers were discarded, and the CH₂Cl₂ layer was dried (MgSO₄), filtered, and concentrated. The resulting yellow oil was purified by silica gel chromatography (3/7 EtOAc/hexane) to provide 285 mg of compound 104: ¹H NMR CDCl₃ (δ) 1.25 (t, 3H), 1.42 (m, 2H), 1.50 (s, 9H), 1.65 (m, 4H), 2.30 (t, 2H), 3.83 (t, 2H), 4.12 (q, 2H), 7.28 (s, 1H); ¹³C NMR CDCl₃ (δ) 14.4, 24.9, 25.6, 27.8, 28.4, 34.3, 60.4, 76.6, 81.7, 157.1, 173.8; HRMS (MALDI-FTMS) calculated for (M+Na) C₁₃H₂₅NaNO₅: 298.1630. Found: 298.1631.

6-(N-tert-butyloxycarbonyl)aminooxyhexanoic acid, compound 105:

To a solution of 1.50 g (5.44 mmol) of compound 104 in 20 mL of EtOH was added 5.44 mL (54.4 mmol) of 10 N NaOH, and the mixture was stirred for 18 hours. The mixture was partitioned between 100 mL of 1 N HCl and four 100 mL portions of CH₂Cl₂. The CH₂Cl₂ layers were combined, dried (MgSO₄), filtered, and concentrated to a yellow oil. Purification by silica gel chromatography (50/50/1 hexane/EtOAc/HOAc) gave 1.22 g (90%) of compound 105 as a colorless oil: ¹H NMR CDCl₃ (δ) 1.45 (m, 2H), 1.48 (s, 9H), 1.66 (m, 4H), 2.37 (t, 2H), 3.85 (t, 2H), 7.21 (s, 1H); ¹³C NMR CDCl₃ (δ) 24.6, 25.5, 27.8, 28.4, 34.0, 76.6, 82.0, 157.5, 179.3.

N-hydroxysuccinimidyl 6-(N-tert-butyloxycarbonyl)aminooxyhexanoate, compound 106: To a solution of 1.07 g (4.32 mmol) of compound 105 and 497 mg (4.32 mmol) of N-hydroxysuccinimide in 20 mL of CH₂Cl₂ was added 818 mg (1.01 mL, 6.48 mmol) of diisopropylcarbodiimide. The reaction was stirred for 18 hours at room temperature, and 1 mL of HOAc was added. The mixture was stirred for another 3 hours and concentrated under vacuum. The residue was dissolved in 75% EtOAc/hexanes, and insoluble material was removed by filtration. The filtrate was concentrated, and the resulting yellow oil was purified by silica gel chromatography (50/50 EtOAc/hexanes) to give 1.31 g (88%) of compound 106 as a colorless oil: ¹H NMR CDCl₃ (δ) 1.50 (s, 9H), 1.52 (m, 2H), 1.69 (m, 2H), 1.80 (m, 2H), 2.63 (t, 2H), 2.84 (s, 4H), 3.88 (t, 2H), 7.25 (s, 1H); ¹³C NMR CDCl₃ (δ) 24.4, 25.1, 25.6, 27.5, 28.2, 30.8, 76.1, 81.5, 157.3, 168.6, 169.4.

Synthesis of Boc-Protected aminooxyhexanoyl/AHAB/TEG platform, 109: Compound 107 was obtained and converted to compound 108 as previously described (U.S. Pat. No. 5,633,395 reaction scheme 4). To a solution of 50 mg (0.058 mmol) of compound 108 in 1 mL of THF was added 38 μL (37 mg, 0.464 mmol) of pyridine followed by a solution of 120 mg (0.348 mmol) of compound 106 in 1 mL of THF. The mixture was stirred for 18 hours, acidified with 1 N HCl, and partitioned between 15 mL of 1 N HCl and three 15 mL portions of CH₂Cl₂. The combined CH₂Cl₂ layers were dried (MgSO₄), filtered, and concentrated. The resulting oil was purified by silica gel chromatography (step gradient; 95/5 CH₂Cl₂/MeOH to 90/10 CH₂Cl₂/MeOH to 80/20 CH₂Cl₂/MeOH) to provide 25 mg (24%) of compound 109 as a gum: ¹H NMR CDCl₃ (δ) 1.32 (M, 18H), 1.47 (s, 9H), 1.65 (m, 18H), 2.20 (t, 16H), 1.80 (m, 2H), 3.21 (m, 8H), 3.40 (brd s, 16H), 3.68 (m, 8H), 3.82 (t, 8H), 6.52 (t, 2H), 6.60 (t, 2H), 7.13 (t, 2H), 7.21 (t, 2H), 7.88 (s, 1H); mass spectrum (ESI) (M+H) calculated for C₈₄H₁₅₇N₁₄O₂₆: 1777. Found 1778.

Aminooxyhexanoyl/AHAB/TEG platform, 86: The Boc-protecting groups are removed from compound 109 in a manner essentially similar to that described for the preparation of compound 16 to provide 86.

### Example 17

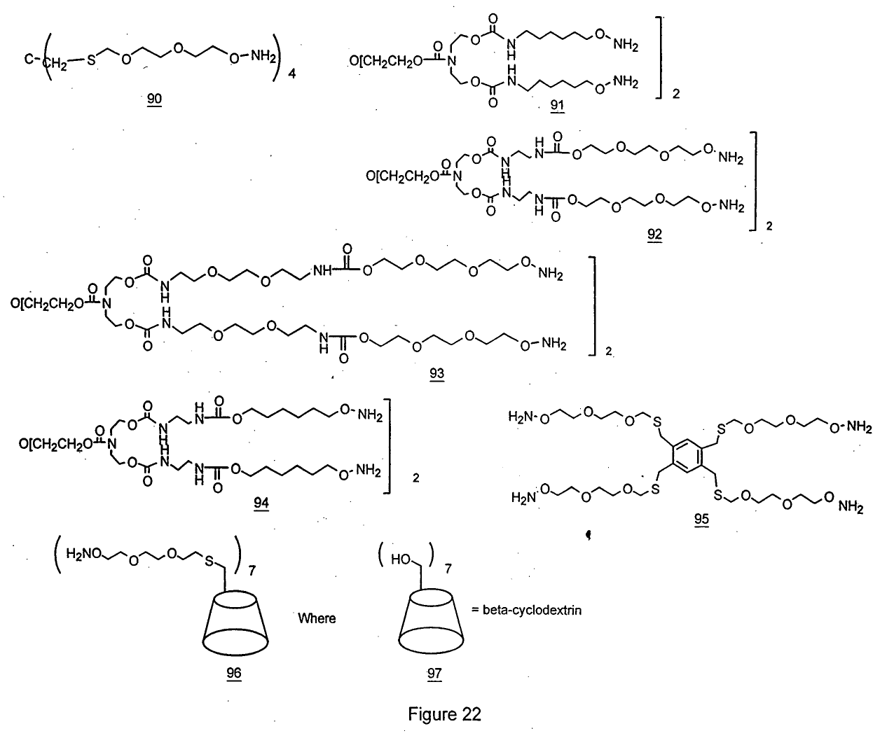

### Synthesis of Compound 91, FIG. 22

Synthesis of 1-azido-6-(N-tert-butyloxycarbonyl)aminooxyhexane, compound 99:

A solution of 300 mg (0.874 mmol) of 1-iodo-6-(N-tert-butyloxycarbonyl)-aminooxyhexane (compound 98 prepared as described by Jones et al., Tetrahedron Letters 2000, 41, 1531-1533.) and 455 mg (7.00 mmol) of sodium azide in 4 mL of DMF was stirred for 72 hours under nitrogen. The mixture was partitioned between 50 mL of EtOAc and three 25 mL portions of H₂O. The EtOAc layer was dried (MgSO₄), filtered, and concentrated. Purification by silica gel chromatography (15/85 EtOAc/hexanes) provided 219 mg (97%) of compound 99 as a colorless oil: ¹H NMR CDCl₃ (δ) 1.41 (m, 4H), 1.49 (s, 9H), 1.63 (m, 4H), 3.28 (t, 2H), 3.83 (t, 2H), 7.22 (s, 1H); ¹³C NMR CDCl₃ (δ) 25.7, 26.7, 28.0, 28.4, 28.9, 51.5, 76.7, 81.7, 157.1.

Synthesis of compound 96: In a reaction vessel equipped with a dry ice condenser, liquid ammonia is added to compound 22a (6.6-8.8 mmol), and the resulting mixture is stirred for 5 min. A per-6-deoxy-6-iodo-cyclodextrin (1 mmol, (Ashton et al., J. Org. Chem. 1996, 61, 903; Gadelle and Defaye, Angew. Chem. Int. Ed. Engl. 1991, 30, 78.) is added. After stirring for 6 h, the ammonia is allowed to evaporate, and the residue is further dried under vacuum and purified by flash column chromatography to provide compound 96.

Synthesis of 1-amino-6-(N-tert-butyloxycarbonyl)aminooxyhexane, compound 100:

A solution of 180 mg (0.697 mmol) of compound 99 and 219 mg (0.836 mmol) of triphenylphosphine in 4 mL of THF and 1 mL of H₂O was stirred for 18 hours at room temperature. There was still starting material present as evidenced by TLC, so another 55 mg (0.209 mmol of triphenylphosphine was added, and the mixture was stirred for 7 hours. The mixture was concentrated and purified by silica gel chromatography (step gradient; Feb. 5, 1993 to Feb. 10, 1988 con NH₄OH/H₂O/CH₃CN) to provide 151 mg of compound 100 as a colorless oil: : ¹H NMR CDCl₃ (δ) 1.35 (m, 4H), 1.49 (s, 9H), 1.61 (m, 4H), 2.69 (t, 2H), 3.82 (t, 2H); ¹³C NMR CDCl₃ (δ) 25.8, 26.7, 28.1, 28.3, 33.2, 41.9, 76.7, 81.3, 157.1.

Synthesis of compound 101: To a solution of 84 mg (81.8 μmol) of compound 14 in 1 mL of CH₂Cl₂ was added a solution of 114 mg (491 μmol) of compound 100 in 0.5 mL of CH₂Cl₂ followed by 86 μL (63 mg, 491 μmol) of diisopropylethylamine. The mixture was stirred for 18 hours at room temperature, quenched with 38 μL (39 mg, 654 μmol) of acetic acid, and concentrated to an oil. Purification by silica gel chromatography (step gradient; 2/98 to 7.5/92.5 MeOH/CH₂Cl₂ provided 115 mg (100%) of 101 as an oil: ¹H NMR CDCl₃ (δ) 1.38 (m, 16H), 1.47 (s, 36H), 1.59 (m, 16H), 3.13 (m, 8H), 3.50 (m, 8H), 3.69 (t, 4H), 3.82 (t, 8H), 4.18 (m, 4H), 4.22 (m, 8H), 5.42 (m, 2H), 5.56 (m, 2H); mass spectrum (ESI) (M+Na) calculated for C₆₂H₁₁₆NaN₁₀O₂₅: 1423. Found 1423.

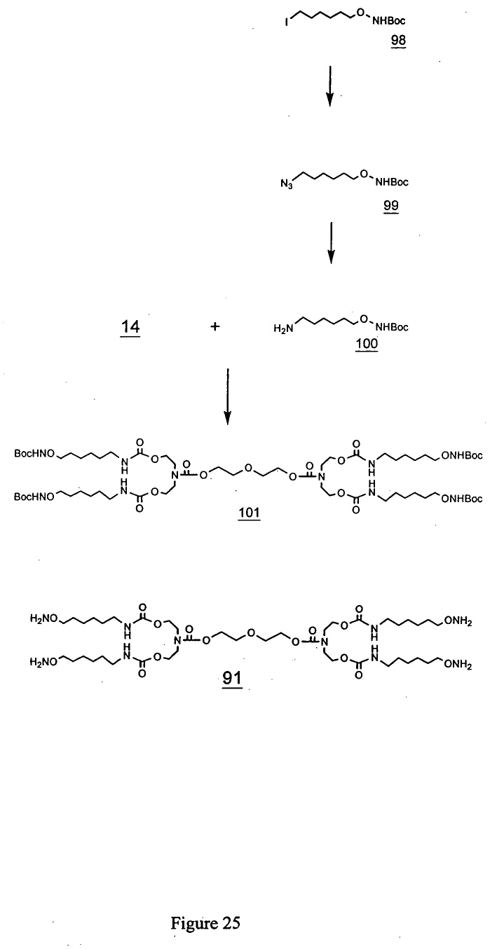

Compound 91: The Boc-protecting groups are removed from compound 101 in a manner essentially similar to that described for the preparation of compound 16 to provide 91. The Reaction Scheme is shown in FIG. 25.

### Example 18

### Synthesis of Compound 92, FIG. 22

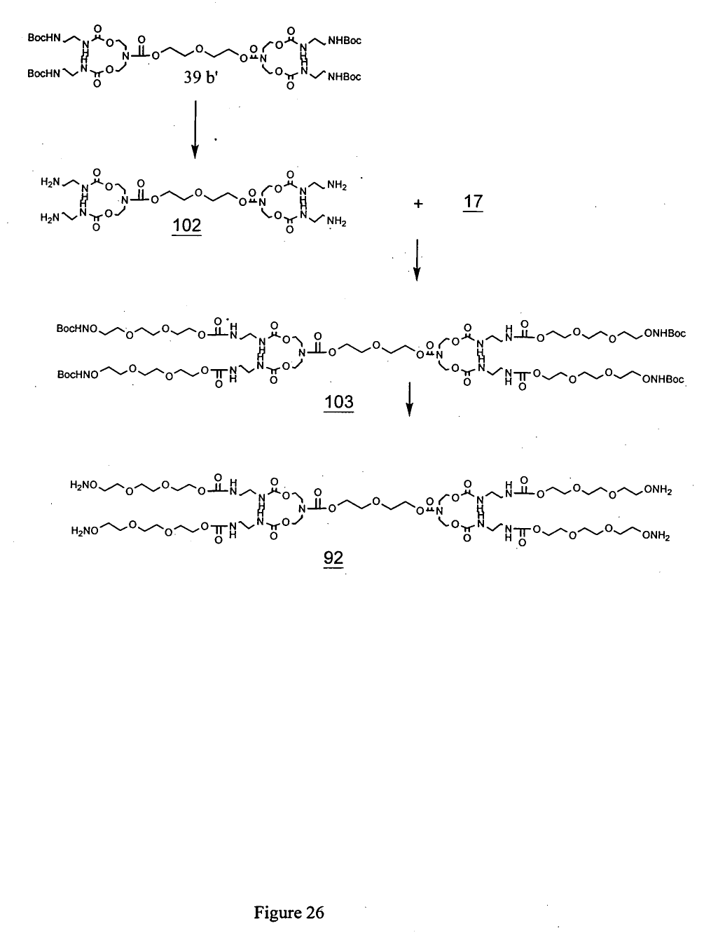

Compound 92 was prepared as described in FIG. 26. The tetra N-Boc-amino platform 39b′ was prepared as described in PCT Application No. PCT/US99/29338; published as PCT Publication No. WO 00/34296, Jun. 15, 2000. Essentially, diethyleneglycol was reacted with para-nitrophenylchloroformate to yield the di para-nitrophenylcarbonate compound, which was then reacted with diethanolamine to form the tetrahydroxy compound, which in turn was reacted with para-nitrophenylchloroformate to yield the tetrapara-nitrophenylcarbonate compound, which in turn was reacted with tert-butyl N-(2-aminoethyl)carbamate to yield 39b′. Compound 39b′ was deprotected with trifluoroacetic acid to provide the tetra-amine, compound 102.

Compound 103: To a solution of 20 mg (0.023 mmol) of compound 102 in 0.5 mL of saturated sodium bicarbonate solution was added a solution of 60 mg (0.140 mmol) of compound 17 in 0.5 mL of dioxane. The mixture was stirred for 5 hours at room temperature, cooled to 0° C., and acidified by dropwise addition of 1 N HCl. The mixture was partitioned between 7 mL of H₂O and four 10 mL portions of CH₂Cl₂. The combined CH₂Cl₂ layers were washed with saturated sodium bicarbonate solution, dried (MgSO₄), filtered, and concentrated. Purification by preparative HPLC (C18, gradient, 30% B to 50% B over 40 min, A=0.1% TFA/H₂O, B=0.1% TFA/CH₃CN) gave 12 mg (27%) of 103 as a viscous oil: ¹H NMR CDCl₃ (δ) 1.48 (s, 36H), 3.26 (m, 16H), 3.51 (m, 8H), 3.68 (m, 44H), 4.02 (m, 8H), 4.21 (m, 12H), 6.12 (brd m, 8H), 8.09 (brd s, 4H); mass spectrum (ESI) (M+Na) calculated for C₇₉H₁₃₆NaN₁₄O₄₁: 1900. Found 1900.

Compound 92: The Boc-protecting groups are removed from compound 103 in a manner essentially similar to that described for the preparation of compound 16 to provide 92. The reaction scheme is shown in FIG. 26.

### Example 19

### Synthesis of Octameric platform 113

To a nitrogen sparged solution of 0.50 g (1.71 mmol) of compound 21b in 8 mL of MeOH at 0° C. was added 537 μL of a 25% solution of NaOMe in MeOH (2.57 mmol), the mixture was stirred at 0° C. for 2 hours, 5.14 mL (5.14 mmol) of nitrogen sparged 1M KHCO₃ solution was added, and the mixture was stirred at 0° C. under nitrogen for 15 minutes. To the mixture was added dropwise a solution of 283 mg (0.14 mmol) of compound 111 (prepared as described in Xeno patent) in 10 mL of 2/1 MeOH/water. The reaction mixture was concentrated to remove MeOH and the concentrate was redissolved in acetonitrile. The reaction mixture was then stirred at room temperature under nitrogen for 3 days, concentrated, and partitioned between 40 mL of EtOAc and 20 mL of water. The EtOAc layer was concentrated, and the product was purified by chromatography on Amberchrom® (70/30 acetonitrile/H₂O to provide 100 mg of compound 112 as a white powder: ¹H NMR (CD₃OD): δ 1.36 (m, 48H), 1.42 (s, 72H), 1.57 (m, 64H), 2.14 (m, 8H), 2.55 (m, 16H), 3.11 (m, 36H), 3.24 (m, 8H), 3.30 (brd s, 16H), 3.71 (t, 16H), 4.2 (m, 4H); ¹³C NMR (CD₃OD): δ 24.31, 25.58, 26.73, 27.77, 28.82, 29.16, 29.73, 29.78, 30.17, 30.24, 30.35, 33.21, 33.69, 36.35, 36.53, 37.17, 38.87, 39.09, 40.43, 40.53, 54.95, 66.07, 70.50, 71.65, 77.44, 82.00, 158.25, 159.20, 172.63, 172.78, 173.97, 176.28; mass spectrum (ESI) (M+2Na)/2 calculated for C₁₆₈H₃₁₂Na₂N₂₆O₄₆S₈: 1866. Found 1866.

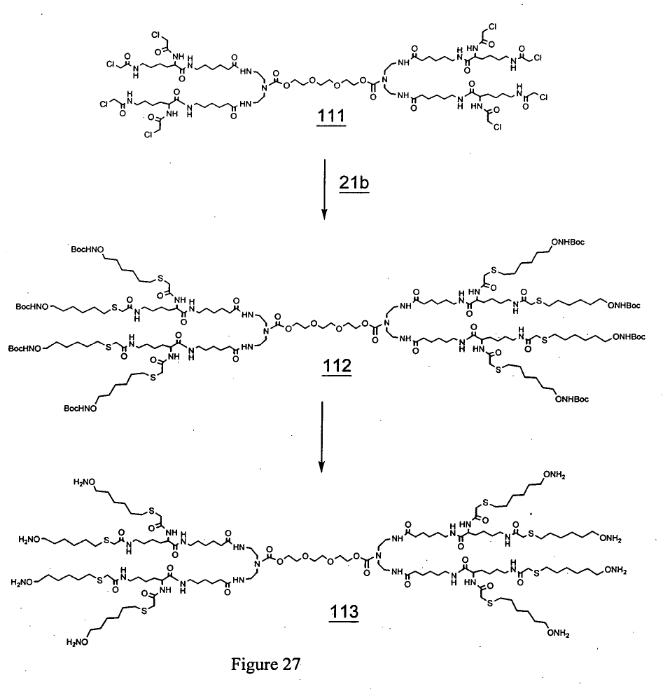

Compound 113: The Boc-protecting groups are removed from compound 112 in a manner essentially similar to that described for the preparation of compound 16 to provide 113. The Reaction Scheme is shown in FIG. 27.

### Example 20

### Synthesis of Compound 125

Compound 115: To a solution of 8.00 g (13.4 mmol) of compound 114 (prepared as described in U.S. Pat. No. 5,552,391) in 80 mL of anhydrous DMF was added 4.00 g (16.1 mmol) of N-(benzyloxycarbonyloxy)succinimide (Aldrich Chemical Co.). The mixture was stirred for 2 hours under nitrogen at room temperature, at which time it was poured into 600 mL of ice water and extracted with four 100 mL portions of CH₂Cl₂. The combined CH₂Cl₂ layers were washed with 100 mL of H₂O, dried (Na₂SO₄), filtered, and concentrated. Concentration from heptane helped to solidify the crude product. Recrystallization from EtOAc gave compound 115 as a white solid: ¹H NMR (CDCl₃) δ 1.26 (m, 4H), 1.43-1.62 (m, 8H), 2.05 (m, 4H), 3.16 (q, 4H), 3.40 (brd s, 8H), 4.98 (s, 2H) overlapped with 5.08 (s, 4H) and 5.11 (s, 2H), 6.31 (s, 1H), 6.44 (s, 1H), 7.26-7.38 (m, 15H).

Synthesis of triamine, compound 116: A solution of 9.0 g (12.3 mmol) of compound 115 in 18 mL of cyclohexane and 36 mL of anhydrous ethanol was deoxygenated by bubbling N₂ gas through it. To the solution was added 1.80 g of 10% Pd/C, and the mixture was heated at reflux for 3 hours. When cool, the mixture was filtered through Celite® using MeOH to rinse. The filtrate was concentrated, and the concentrate was concentrated from CH₂Cl₂ to provide 4.20 g (87%) of compound 116 as an off white solid.

Synthesis of compound 117: To a solution of 5.39 g (21.8 mmol) of compound 105 in 10 mL of anhydrous acetonitrile was added 3.02 g (23.9 mmol) of CDI (carbonyldiimidazole), and the mixture was stirred for 1.5 hours under nitrogen atmosphere. The resulting solution was added to a solution of 4.20 g (10.7 mmol) of compound 116 in 15 mL of anhydrous DMF, and the mixture was stirred for 2 hours and poured into 500 mL of ice water. The resulting mixture was extracted with four 100 mL portions of CH₂Cl₂. The combined CH₂Cl₂ layers were washed with 100 mL of H₂O, dried (Na₂SO₄), filtered, and concentrated. The resulting semisolid residue was crystallized from 10% isopropyl alcohol/EtOAc to provide 4.0 g (44%) of 117 as a white solid: ¹H NMR CDCl₃ (δ) 1.35 (m, 4H), 1.42 (m, 4H), 1.49 (s, 18H), 1.63 (m, 16H), 2.01 (brd s, 1H), 2.20 (t, 4H), 3.23 (m, 4H), 3.34 (m, 4H), 3.85 (t, 4H), 6.34 (t, 2H), 6.70 (t, 2H), 7.98 (brd s, 1H).

Compound 119: To a solution of 3.65 g (14.11 mmol) of 9-fluorenylmethylchloroformate (Fmoc-Cl) in 15 mL of dioxane was added a solution of 3.00 g (15.5 mmol) of compound 118 (Bondunov et al., J. Org. Chem. 1995, Vol. 60, pp. 6097-6102) in 15 mL of dioxane followed by a solution of 1.95 g (14.11 mmol) of potassium carbonate in 30 mL of H₂O. The mixture was stirred for 18 hours at room temperature and concentrated. The resulting oil was partitioned between 50 mL of 1 N NaOH solution and three 150 mL portions of CH₂Cl₂. The combined CH₂Cl₂ layers were dried (MgSO₄), filtered, and concentrated to a yellow oil. Purification by silica gel chromatography (step gradient; 90/10 EtOAc/AcOH to 90/10/1 EtOAc/AcOH/MeOH) to give 3.85 g (66%) of 119 as a viscous oil: ¹H NMR CDCl₃ (δ) 3.26 (m, 4H), 3.39 (m, 2H), 3.49 (m, 2H), 3.59 (m, 2H), 3.65 (m, 4H), 3.69 (m, 2H), 4.25 (t, 1H), 4.60 (d, 2H), 7.35 (t, 2H), 7.41 (t, 2H), 7.59 (d, 2H), 7.78 (d, 2H).

Compound 120: To a solution of 3.77 g (9.08 mmol) of compound 119 and 7.32 g (36.3 mmol) of 4-nitrophenylchloroformate in 50 mL of CH₂Cl₂ at 0° C. was added 5.88 mL (5.75 g, 72.6 mmol) of pyridine. The mixture was stirred at room temperature under nitrogen atmosphere for 72 hours, and the mixture was partitioned between 200 mL of CH₂Cl₂ and four 100 ml portions of 10% aqueous sodium bicarbonate solution. The CH₂Cl₂ layer was washed successively with 100 mL of H₂O, 100 mL of 1 N HCl, then 100 mL of brine. The solution was dried (MgSO₄), filtered, and concentrated to yield an orange oil. Purification by silica gel chromatography (15/50/35/1 EtOAc/CH₂Cl₂/hexane/AcOH) to provide 2.67 g (39%) of compound 120 as a yellow gum: ¹H NMR (CDCl₃) δ 3.32 (m, 4H), 3.52 (m, 2H). 3.60 (m, 4H), 3.74 (m, 2H), 4.23 (t, 1H), 4.38 (m, 2H), 4.41 (m, 2H), 4.57 (d, 2H), 7.37 (m, 8H), 7.59 (d, 2H), 7.78 (d, 2H), 8.26 (overlapping d, 4H); mass spectrum (ESI) (M+H) calculated for C₃₇H₃₆N₃O₁₄: 746. Found 746.

Compound 121: To a solution of 482 mg (0.612 mmol) of compound 117 in 5 mL of CH₂Cl₂ was added 182 mg (0.245 mmol) of compound 120 followed by 171 μL (124 mg, 1.22 mmol) of Et₃N and 26 mg (0.490 mmol) of HOBt. The mixture was stirred at room temperature until the reaction was complete as judged by TLC (1/9 MeOH/CH₂Cl₂). The mixture was partitioned between 300 mL of CH₂Cl₂ and three 50 mL portions of 1 N HCl. The CH₂Cl₂ layer was washed with brine, dried (MgSO₄), filtered, and concentrated to a yellow oil. Purification by silica gel chromatography (multiple step gradient; May 1, 1994 to Oct. 1, 1989 to 15/1/84 to 20/1/79 MeOH/HOAc/CH₂Cl₂) to provide 317 mg (63%) of compound 121 as a sticky white solid: ¹H NMR (CD₃OD) δ 1.34 (m, 16H), 1.43 (m, 8H), 1.48 (s, 36H), 1.64 (m, 24H), 2.20 (m, 16H), 3.19 (m, 12H), 3.25-3.52 (m, 18H), 3.55 (m, 2H), 3.79 (t, 8H), 4.16 (m, 4H), 4.28 (t, 1H), 4.59 (d, 2H), 7.33 (t, 2H), 7.41 (t, 2H), 7.60 (d, 2H), 7.84 (d, 2H); ¹³C NMR (CD₃OD) δ 14.6, 23.8, 26.7, 26.7, 26.9, 27.7, 28.8, 28.9, 30.3, 37.1, 38.8, 39.1, 40.3, 65.8, 66.0, 68.1, 70.2, 70.3, 77.3, 82.0, 121.2, 126.0, 128.4, 129.0, 142.9, 145.6, 157.9, 158.2, 159.2, 176.1, 176.3; mass spectrum (ESI) (M+2Na)/2 calculated for C₁₀₁H₁₇₁Na₂N₁₅O₂₈: 1044. Found 1044.

Compound 122: To a solution of 163 mg (79.8 mmol) of compound 121 in 2.4 mL of DMF was added 600 μL of diethylamine. The mixture was stirred for 3 hours and concentrated. Purification by silica gel chromatography (multi-step gradient; Oct. 1, 1989 to 12.5/6/86.5/to 15/1/84 MeOH/con NH₄OH/CH₂Cl₂) gave 127 mg (81%) of compound 122 as a glassy gum: ¹H NMR (CD₃OD) δ 1.38 (m, 16H), 1.48 (m, 44H), 1.65 (m, 24H), 2.20 (t, 16H), 2.83 (t, 4H), 3.17 (t, 8H), 3.38 (m, 16H), 3.63 (t, 4H), 3.69 (t, 4H), 3.78 (t, 4H), 4.21 (m, 4H); ¹³C NMR (CD₃OD) δ 26.7, 27.0, 27.8, 28.8, 28.9, 30.3, 37.1, 38.8, 39.1, 40.3, 49.9, 66.0, 70.4, 70.9, 77.3, 82.0, 158.2, 159.2, 176.1, 176.3; mass spectrum (ESI) (M+H) calculated for C₈₆H₁₆₂N₁₅O₂₆: 1821. Found 1821.

Compound 124b: To a solution of 20 mg (11.0 μmol) of compound 122 in 5 mL of DMF was added 103 mg (8.8 μmol) of methoxypolyethyleneglycol benzotriazolylcarbonate of molecular weight 11,690 g/mol (mPEG_{12K}-BTC, compound 123b, Shearwater Polymers) followed by 5 μL (3.6 mg, 35.9 μmol) of Et₃N. The mixture was stirred at room temperature for 18 hours and concentrated. The residue was purified by silica gel chromatography (multi-step gradient; 5/95 to 15/85 to 20/80 MeOH/CH₂Cl₂) to provide 109 mg of compound 124b as a waxy off white solid: ¹H NMR (CDCl₃) δ 1.37 (m, 16H), 1.49 (m, 44H), 1.65 (m, 24H), 2.20 (t, 16H), 3.20 (q, 8H), 3.36 (m, 16H), 3.61 (m, 4H), 3.68 (m, approximately 1056H), 3.84 (t, 8H), 3.91 (m, 4H), 4.23 (m, 4H).

Compound 124a: This compound was prepared using essentially the same procedure used for the preparation of compound 124b; however, methoxypolyethyleneglycol benzotriazolylcarbonate of molecular weight 5,215 g/mol (mPEG_{5K}-BTC, compound 123a, Shearwater Polymers) was used: ¹H NMR (4:1 CDCl₃/CD₃OD) δ 1.37 (m, 16H), 1.49 (m, 44H), 1.65 (m, 24H), 2.20 (t, 16H), 3.20 (q, 8H), 3.36 (m, 16H), 3.61 (m, 4H), 3.68 (m, approximately 468H), 3.84 (t, 8H), 3.91 (m, 4H), 4.23 (m, 4H).

Compound 124c: This compound was prepared using essentially the same procedure used for the preparation of compound 124b; however, methoxypolyethyleneglycol benzotriazolylcarbonate of molecular weight 22,334 g/mol (mPEG_{20K}-BTC, compound 123c, Shearwater Polymers) was used: ¹H NMR (5:1 CDCl₃/CD₃OD) δ 1.37 (m, 16H), 1.49 (m, 44H), 1.65 (m, 24H), 2.20 (t, 16H), 3.20 (q, 8H), 3.36 (m, 16H), 3.61 (m, 4H), 3.68 (m, approximately 2024H), 3.84 (t, 8H), 3.91 (m, 4H), 4.23 (m, 4H).

Compound 125b: The Boc-protecting groups are removed from compounds 124a-c in a manner essentially similar to that described for the preparation of compound 16 to provide compounds 125a-c.

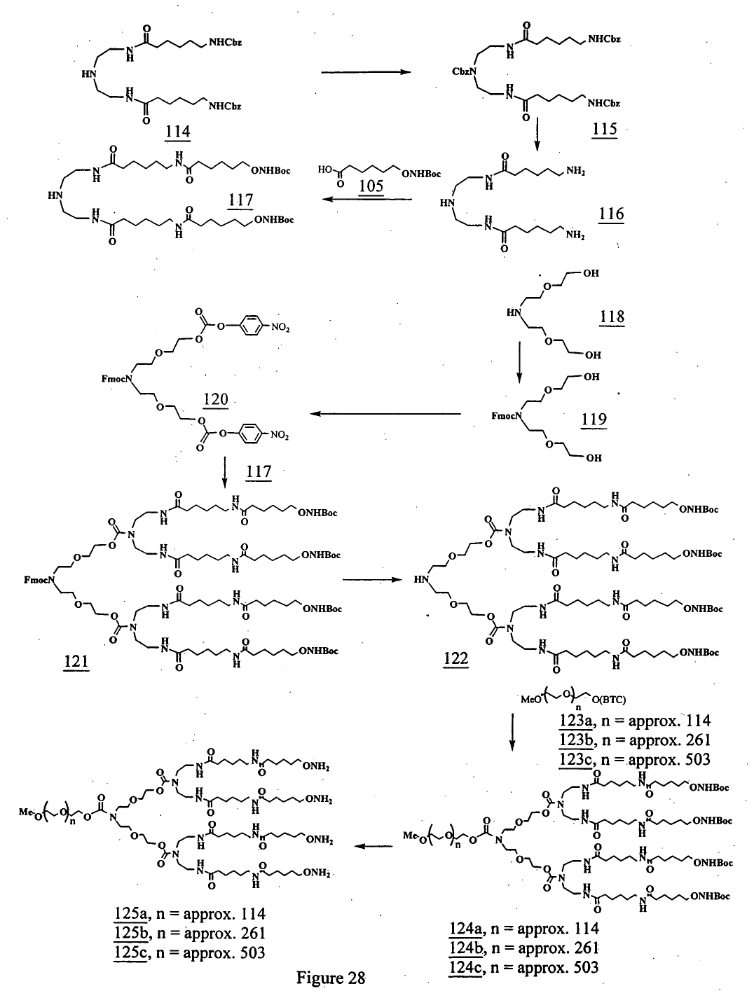

The reaction scheme is shown in FIG. 28.

### Example 21

### Synthesis of Compound 129

Compound 126: To a solution of 14 mg (18.6 μmol) of compound 120 and 29 mg (186.3 μmol) of HOBT in 5 mL of anhydrous DMF was added 56 μL (38 mg, 373 μmol) of Et₃N. The mixture was stirred for 1 hour and a solution of 85 mg (46.6 μmol) of compound 122 in 1 mL of DMF was added. The mixture was stirred at room temperature for 5 hours and partitioned between 150 mL of CH₂Cl₂ and 50 mL of 1 N HCl. The CH₂Cl₂ layer was washed with brine, dried (MgSO₄), filtered, and concentrated. Purification by silica gel chromatography provided 34 mg (44%) of compound 126 as a waxy white solid: ¹H NMR (CD₃OD) δ 1.37 (m, 32H), 1.49 (m overlapping s at 1.48, 88H) 1.62 (m, 48H), 2.20 (t, 32H), 3.18 (t, 16H), 3.36 (m, 32H), 3.50 (m, 12H), 3.64 (m, 24H), 3.79 (t, 16H) 4.17 (m, 12H), 4.29 (t, 1H), 4.60 (d, 2H), 7.37 (t, 2H), 7.43 (t, 2H), 7.65 (d, 2H), 7.84 (d, 2H); mass spectrum (ESI) (M+3Na)/3 calculated for C₁₉₇H₃₄₇Na₃N₃1O₆₀: 1393. Found 1393.

Compound 127: To a solution of 34 mg (8.27 mmol) of compound 126 in 1.6 mL of DMF was added 400 μL of diethylamine. The mixture was stirred at room temperature for 4 hours and concentrated. The concentrate was purified by silica gel chromatography (Jan. 10, 1989 con NH₄OH/MeOH/CH₂Cl₂) to provide 13 mg (40%) of compound 127: ¹H NMR (CD₃OD) δ 1.35 (m, 32H), 1.49 (m overlapping s at 1.48, 88H), 1.63 (m, 48H), 2.19 (t, 32H), 3.08 (brd t, 4H) 3.17 (t, 16H), 3.38 (m, 36H), 3.52 (m, 8H), 3.63 (t, 8H), 3.70 (m, 12H), 3.78 (t, 16H), 4.21 (m, 12H); mass spectrum (ESI) (M+3Na)/3 calculated for C₁₈₂H₃₃₇Na₃N₃]O₅₈: 1319. Found 1319.

Compound 128: To a solution of 13 mg (3.34 mmol) of compound 127 in 5 mL of pyridine was added 60 mg (2.68 μmol) of methoxypolyethyleneglycol benzotriazolylcarbonate of molecular weight 22,334 g/mol (mPEG_{20K}-BTC, Shearwater Polymers) followed by 5 μL (3.6 mg, 35.9 μmol) of Et₃N. The mixture was stirred at room temperature for 18 hours and concentrated. The residue was purified by silica gel chromatography (multi-step gradient; 10/90 to 15/85 to 20/80 MeOH/CH₂Cl₂) to provide 45 mg of compound 128 as a waxy solid: ¹H NMR (CDCl₃) δ 1.30 (m, 32H), 1.50 (m overlapping s at 1.48, 88H), 1.67 (m, 48H), 2.24 (t, 32H), 3.23 (m, 16H), 3.41 (m, 32H), 3.65 (m, approximately 2024H), 3.70 (t, 24H), 3.89 (m, 16H), 4.21 (m, 12H).

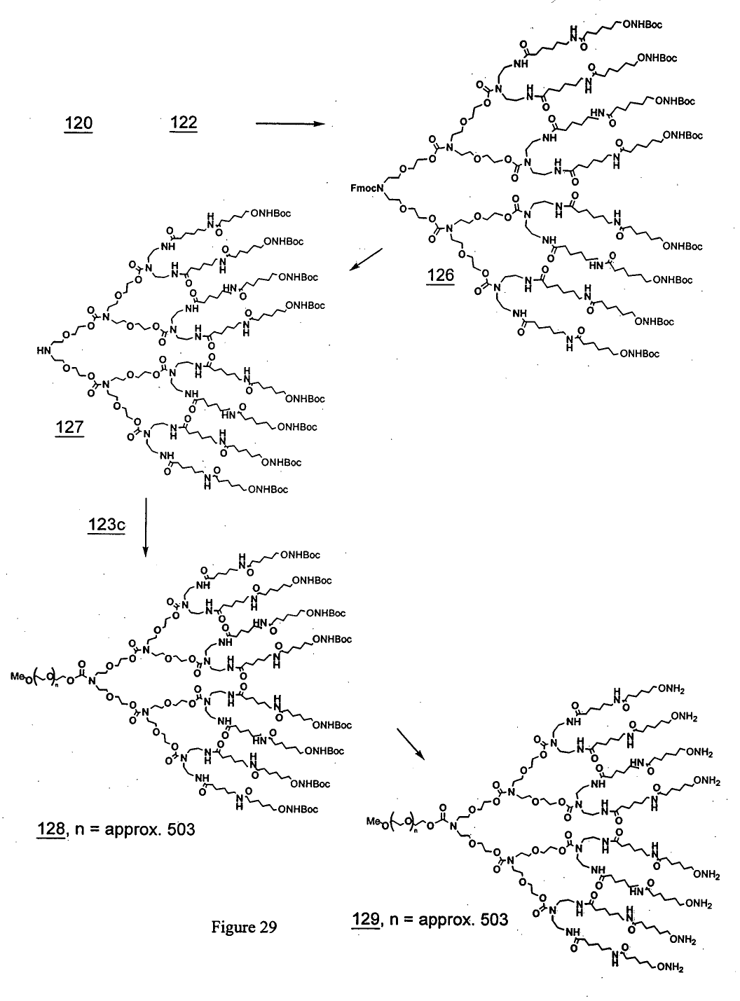

Compound 129: The Boc-protecting groups are removed from compound 128 in a manner essentially similar to that described for the preparation of compound 16 to provide compounds 129, as shown in FIG. 29.

### Example 22

### Synthesis of Compound 132

Compound 131: To a solution of 22 mg (27.3 μmol) of compound 117 in 5 mL of pyridine was added 236 mg (10.9 μmol) of polyethyleneglycol bis-benzotriazolylcarbonate of molecular weight 21,529 g/mol (PEG_{20K}-bis-BTC, compound 130, Shearwater Polymers) followed by 8 μL (5.8 mg, 57.4 μmol) of Et₃N. The mixture was stirred at room temperature for 18 hours and concentrated. The residue was purified by silica gel chromatography (multi-step gradient; 5/95 to 10/90 to 15/85 to 20/80 MeOH/CH₂Cl₂) to provide 242 mg (96%) of compound 131 as a white solid: ¹H NMR (CDCl₃) δ 1.35 (m, 16H), 1.48 (m, 44H), 1.61 (m, 24H), 2.20 (m, 16H), 3.22 (m, 8H), 3.52-3.96 (m, approximately 2000H), 4.23 (m, 4H).

Compound 132: The Boc-protecting groups are removed from compound 131 in a manner essentially similar to that described for the preparation of compound 16 to provide compound 132.

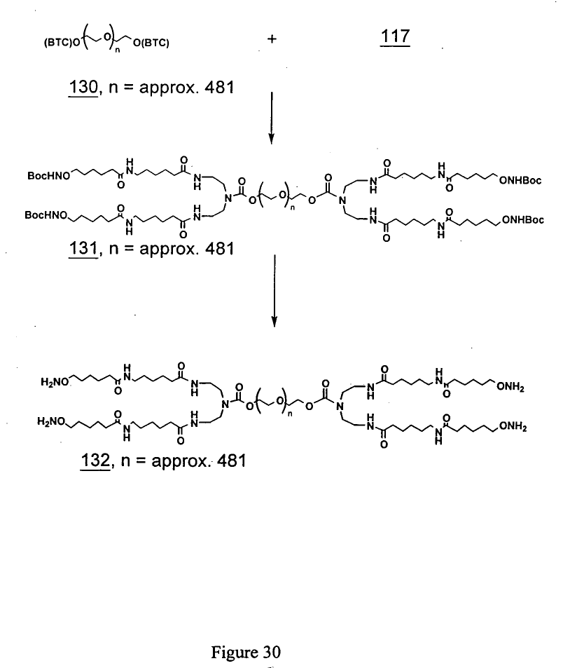

The reaction scheme is shown in FIG. 30.

### Example 23

### Synthesis of Compound 136

Compound 134: To a solution of 3.87 mg (4.85 μmol) of pentaerythritol tetrakis-(4-nitrophenylcarbonate ester) (prepared by reaction of pentaerythritol with para-nitrophenylchloroformate to yield the tetrapara-nitrophenylcarbonate compound) in 5 mL of pyridine was added 124 mg (24.2 μmol) of mono-Boc-protected diaminopolyethylene glycol of molecular weight 5094 g/mol (compound 133, BocNH-PEG_{(5K)}-NH₂), and 5 μL (3.63 mg, 35.9 mmol) of Et₃N. The mixture was stirred for 18 hours and concentrated. The residue was purified by silica gel chromatography (step gradient; 5/95 to 15/85 MeOH/CH₂Cl₂) to provid 77 mg (77%) of compound 134 as a white solid: ¹H NMR (CDCl₃) δ 1.48 (s, 36H), 3.32 (m, 16H), 3.52-3.96 (m, approximately 1818H), 4.10 (m, 8H).

Compound 135: Compound 134 (77 mg, 3.73 μmol) was dissolved in 5 mL of trifluoroacetic acid, and the mixture was allowed to stand for three hours. The TFA was removed under a stream of N₂ and the residue was dissolved in 5 mL of CH₂Cl₂. To the resulting solution was added a solution of 7.72 mg (22.4 μmol) of compound 106 in 5 mL of CH₂Cl₂ followed by 35 μL (25.4 mg, 251 μmol) of Et₃N. (Note: The pH of the mixture should be checked and adjusted accordingly with Et₃N to make sure it is basic.) The mixture was stirred under nitrogen for 18 hours. The mixture was partitioned between 50 mL of CH₂Cl₂ and three 25 mL portions of 1 N HCl. The CH₂Cl₂ layer was washed with brine, dried (MgSO₄), filtered and concentrated. Purification by silica gel chromatography (step gradient; 5/95 to 10/90 MeOH/CH₂Cl₂) provided 42 mg (53%) of compound 135 as waxy solid: ¹H NMR (CDCl₃) δ 1.40 (m, 8H), 1.48 (s, 36H), 1.66 (m, 16H), 2.18 (t, 8H), 3.32 (m, 16H), 3.38-3.89 (m, approximately 1818H), 4.10 (m, 8H), 4.97 (t, 4H), 6.43 (t, 4H), 7.47 (s, 4H).

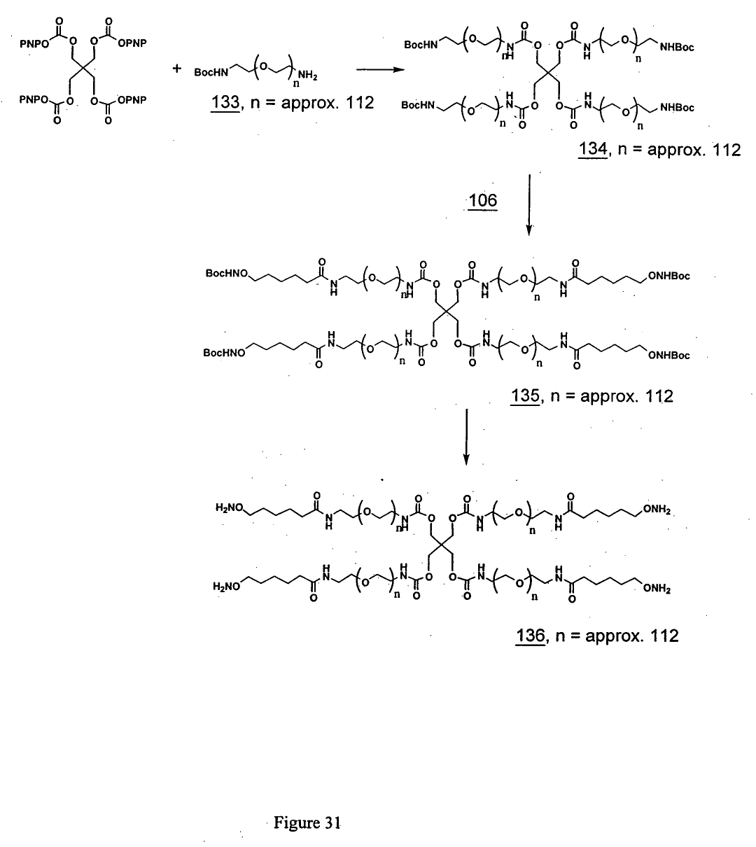

Compound 136: The Boc-protecting groups are removed from compound 135 in a manner essentially similar to that described for the preparation of compound 16 to provide compound 136, as shown in FIG. 31.

### Example 24

### Synthesis of Compound 143

Compound 137: To a 0° C. solution of 200 mg (1.11 mmol) of ethyl 3,5-diaminobenzoate in 5 mL of CH₂Cl₂ under nitrogen atmosphere was added 928 μL (674 mg, 6.66 mmol) of Et₃N. To the mixture was added dropwise a solution of 510 μL (710 mg, 3.33 mmol) of 6-bromohexanoyl chloride in 5 mL of CH₂Cl₂. The mixture was stirred at room temperature for 1.5 hours and partitioned between 50 mL of 1 N HCl and two 50 mL portions of CH₂Cl₂. The CH₂Cl₂ layers were washed with saturated sodium bicarbonate solution, dried (MgSO₄), filtered and concentrated. The product was purified by silica gel chromatography (6/4 hexane/EtOAc) to give 554 mg (93%) of compound 137 as an oil: ¹H NMR (CDCl₃): δ1.39 (t, 3H), 1.52 (m, 4H), 1.75 (m, 4H), 1.90 (m, 4H), 2.40 (t, 4H), 3.42 (t, 4H), 4.36 (q, 2H), 7.60 (s, 2H), 7.88 (s, 2H), 8.17 (s, 1H).

Compound 138: DBU (612 μL, 623 mg, 4.01 mmol) was added to a solution of 547 mg (1.02 mmol) of compound 137 and 272 mg (2.05 mmol) of N-(tert-butyloxycarbonyl)hydroxylamine (Aldrich Chemical Co.). The mixture was stirred for 18 hours at room temperature and partitioned between 50 mL of 1 N HCl and three 50 mL portions of CH₂Cl₂. The combined CH₂Cl₂ layers were dried (MgSO₄), filtered, and concentrated. The product was purified by silica gel chromatography (1/1 hexane/EtOAc) to give 216 mg (33%) of compound 138 as a white solid: mp 55-60° C.; ¹H NMR (CDCl₃): δ 1.38 (t, 3H), 1.48 (s, 18H; buried m, 4H), 1.60 (m, 4H), 1.73 (m, 4H), 2.40 (m, 4H), 3.86 (t, 4H), 4.36 (q, 2H), 7.41 (s, 2H), 7.90 (s, 2H), 8.06 (s, 2H), 8.11 (s, 1H); mass spectrum (ESI) (M+Na) calculated for C₃₁H₅₀NaN₄O₁₀: 661. Found 661.

Compound 139: To a solution of 205 mg (0.32 mmol) of compound 138 in 1/1 acetone/EtOH was added 256 μL (2.56 mmol) of 10 N NaOH, and the mixture was heated to 60° C. for 4 hours. When cool, the mixture was partitioned between 50 mL of 1 N HCl and four 50 mL portions of 4/1 CH₂Cl₂/MeOH. The combined organic layers were dried (MgSO₄), filtered, and concentrated. The product was purified by silica gel chromatography (3/97/1 MeOH/CH₂Cl₂/HOAc) to give 184 mg (94%) of compound 139 as a viscous oil: ¹H NMR (CDCl₃): δ 1.38 (m, 4H), 1.42 (s, 18H), 1.60 (m, 4H), 1.70 (m, 4H), 2.38 (m, 4H), 3.80 (t, 4H), 7.77 (s, 2H), 8.00 (s, 2H), 8.11 (s, 1H), 8.91 (s, 2H); mass spectrum (ESI) (M+Na) calculated for C₂₉H₄₆NaN₄O₁₀: 633. Found 633.

Compound 140: To a 0° C. solution of 164 mg (0.268 mmol) of compound 139 in 2.0 mL of dry THF was added 31 mg (0.268 mmol) of N-hydroxysuccinimide, followed by 83 mg (0.403 mmol) of DCC. The mixture was allowed to come to room temperature and stirred for 18 hours under nitrogen atmosphere, and 200 μL of HOAc was added. The mixture was stirred for another hour, diluted with approximately 5 mL of EtOAc, and allowed to stand for an hour. The resulting precipitate was removed by filtration, and the filtrate was concentrated. Purification by silica gel chromatography (3/97/MeOH/CH₂Cl₂) provided 129 mg (68%) of compound 140 as a white solid: ¹H NMR (CDCl₃): δ 1.40 (m, 4H), 1.43 (s, 18H), 1.65 (m, 4H), 1.80 (m, 4H), 2.34 (m, 4H), 2.93 (s, 4H), 3.85 (t, 4H), 7.68 (s, 2H), 7.87 (s, 2H), 8.36 (s, 1H), 8.61 (s, 2H).

Compound 142: To a solution of 60 mg (0.85 mmol) of compound 140 in 0.5 mL of CH₂Cl₂ was added 14 μL (13.3 mg, 0.168 mmol) of pyridine. The mixture was cooled to 0° C. and a solution of 71 mg (0.021 mmol) of diamino-PEG, compound 141, in 0.5 mL of CH₂Cl₂ was added. The mixture was stirred under nitrogen atmosphere at room temperature for 18 hours, and partitioned between 10 mL of 1 N HCl and three 10 mL portions of CH₂Cl₂. The combined CH₂Cl₂ layers were dried (MgSO₄), filtered, and concentrated. Purification by silica gel chromatography (step gradient 5/95 MeOH/CH₂Cl₂ to 10/90 MeOH/CH₂Cl₂) provided 66 mg (69%) of compound 142 as a viscous oil: ¹H NMR (CDCl₃): δ 1.45 (s, 36H), 1.60-1.80 (m, 24H), 2.39 (t, 8H), 3.39 (m, 8H), 3.50-3.80 (brd s, approx. 318H), 3.87 (t, 8H), 4.22 (t, 4H), 7.50 (brd s, 2H), 7.63 (s, 4H), 7.77 (s, 2H), 8.08 (s, 2H), 8.60 (s, 2H); mass spectrum (MALDI) (M+H) calculated for C₂₀₇H₃₈₉N₁₂O₉₃: 4535. Found distribution centered at approximately 4324.

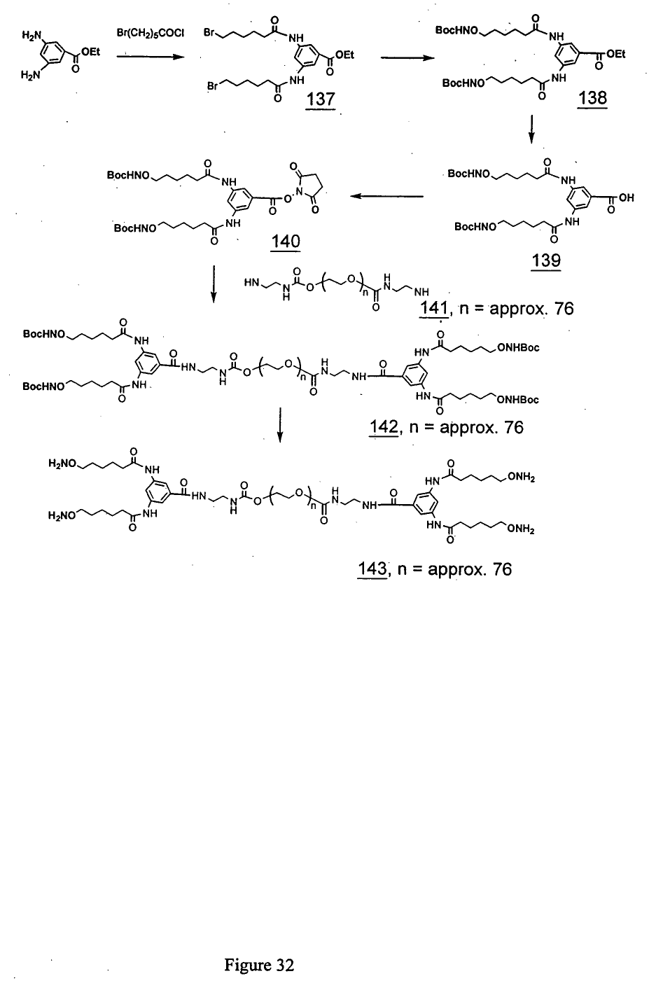

Compound 143: The Boc-protecting groups are removed from compound 142 in a manner essentially similar to that described for the preparation of compound 16 to provide 143, as shown in FIG. 32.

### Example 25

### Method of Preparation of Conjugates

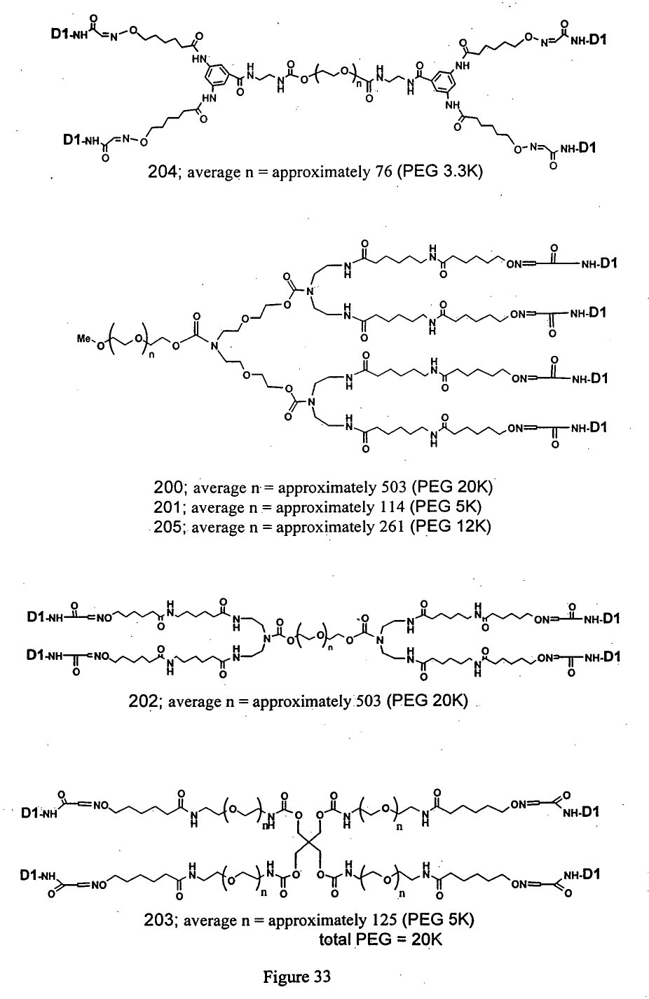

Conjugates 200, 201, 202, 203, 204, and 205 (FIG. 33) were prepared as follows.

Compound 200: To a solution of 68.8 mg (9.74 μmol, 6 equivalents) of TA/D1 in 10 mL of helium sparged 0.1 M, pH 4.6 sodium acetate buffer was added a solution of 36.8 mg (1.62 μmol) of compound 125c in 6.15 mL of 1/1 acetonitrile/0.1 M, pH 8.0 tris acetate buffer. Care was taken to keep the mixture under nitrogen atmosphere while stirring at room temperature for 18 hours. When the reaction was complete, it was directly purified by cation exchange chromatography using a PolyCat A WCX column manufactured by PolyLC Inc. (gradient 10% B to 25% B, A=10 mM sodium phosphate pH 7 in 1/9 acetonitrile/H₂O) to provide 57 mg (40%) of compound 200.

Compound 201: Compound 201 was prepared in a manner essentially similar to compound 200. Thus, to an approximately 1 mM solution of 6 equivalents of TA/D1 in helium sparged 0.1 M, pH 4.6 sodium acetate buffer was added 1 equivalent of compound 125a as a 0.25 to 10 mM solution in 1/1 acetonitrile/0.1 M, pH 8.0 tris acetate buffer. Care was taken to keep the mixture under nitrogen atmosphere while stirring at room temperature for 18 hours. When the reaction was complete, it was directly purified by cation exchange chromatography to provide compound 201.

Compound 202: Compound 202 was prepared in a manner essentially similar to 200. Thus, to an approximately 1 mM solution of 6 equivalents of TA/D1 in helium sparged 0.1 M, pH 4.6 sodium acetate buffer was added 1 equivalent of compound 132 as a 0.25 to 10 mM solution in 1/1 acetonitrile/0.1 M, pH 8.0 tris acetate buffer. Care was taken to keep the mixture under nitrogen atmosphere while stirring at room temperature for 18 hours. When the reaction was complete, it was directly purified by cation exchange chromatography to provide compound 202.

Compound 203: Compound 203 was prepared in a manner essentially similar to 200. Thus, to an approximately 1 mM solution of 6 equivalents of TA/D 1 in helium sparged 0.1 M, pH 4.6 sodium acetate buffer was added 1 equivalent of compound 136 as a 0.25 to 10 mM solution in 1/1 acetonitrile/0.1 M, pH 8.0 tris acetate buffer. Care was taken to keep the mixture under nitrogen atmosphere while stirring at room temperature for 18 hours. When the reaction was complete, it was directly purified by cation exchange chromatography to provide compound 203.

Compound 204: Compound 204 was prepared in a manner essentially similar to 200. Thus, to an approximately 1 mM solution of 6 equivalents of TA/D1 in helium sparged 0.1 M, pH 4.6 sodium acetate buffer was added 1 equivalent of compound 143 as a 0.25 to 10 mM solution in 1/1 acetonitrile/0.1 M, pH 8.0 tris acetate buffer. Care was taken to keep the mixture under nitrogen atmosphere while stirring at room temperature for 18 hours. When the reaction was complete, it was directly purified by cation exchange chromatography to provide compound 204.

Compound 205: Compound 205 was prepared in a manner essentially similar to 200. Thus, to an approximately 1 mM solution of 6 equivalents of TA/D1 in helium sparged 0.1 M, pH 4.6 sodium acetate buffer was added 1 equivalent of compound 125b as a 0.25 to 10 mM solution in 1/1 acetonitrile/0.1 M, pH 8.0 tris acetate buffer. Care was taken to keep the mixture under nitrogen atmosphere while stirring at room temperature for 18 hours. When the reaction was complete, it was directly purified by cation exchange chromatography to provide compound 205.

### Example 26

### Evaluation of Toleragen Efficiency and Serum Half-Life

Domain 1—keyhole limpet hemocyanin conjugate (D1-KLH) was prepared for use in animal immunization. Recombinant Domain 1 with a fifth cysteine was expressed as a glutathione mixed disulfide in insect cells using the baculovirus expression vector system. The structure consists of the first 66 amino-terminal amino acids present in native human β₂-glycoprotein I followed by a C-terminal leu-(his)₅ expression tag. The polyhistidine expression tag at the C-terminus was the basis for a purification procedure by nickel affinity chromatography. Iverson et al. (1998) Proc. Nat'l. Acad. Sci. 95: 15542-15546.

The resulting Domain 1 with a free sulfhydryl (D1-SH) was alkylated by maleimidyl-KLH. Maleimidyl-activated KLH (Pierce Chemical Co.; Rockford, Ill.) was dissolved at 10 mg/mL in water as per the manufacturer's instructions. Immediately, the KLH was added to D1-SH at a ratio of 1.27 mg per mg D1-SH. The tube containing the KLH and D1 was mixed by rotation at 2 h×RT. At the end of the incubation the contents were dialyzed against cold PBS at 4° C. using a >25,000 MW cut-off membrane for the removal of unconjugated D1. An aliquot of the dialyzed sample was removed and tested for the presence of immunoreactive D1 by an ELISA with patient-derived affinity purified antiphospholipid antibodies (aPL).

An immunized rat model was used for measuring toleragen efficacy. Lewis rats (Harlan Sprague Dawley, Indianapolis, Ind.) were immunized i.p. with 101 g of D1-KLH in alum with pertussis adjuvant. Three weeks after priming, groups of four animals were treated i.v. with toleragen or PBS control. Five days after treatment animals were boosted i.p. with 10 μg D1-KLH, and sera samples were collected seven days after boost.

An ELISA was used for detection of anti-domain 1 antibody in rat sera. Nunc Maxisorp Immunoplates (Nalge Nunc International, Rochester, N.Y.) were coated overnight with 50 μl of 5 μg/ml recombinant human P₂-GPI in carbonate buffer (Sigma, St. Louis, Mo.) pH 9.6 at 4° C. Subsequent steps were carried out at room temperature. Plates were washed 3× with phosphate buffered saline (PBS), then blocked 1 h with 250 μl 2% nonfat dry milk (Carnation, Solon, Ohio) in PBS. After washing, wells were incubated 1 h with 50 μl serial dilutions in PBS of each sera sample in triplicate. Non-immunized serum was used as control, and a pool of sera from immunized animals was used to generate a standard curve. After washing, the wells were incubated 1 h with 50 μl alkaline phosphatase-conjugated goat anti-rat IgG (Jackson ImmunoResearch, West Grove, Pa.) diluted 1:2000 in PBS/0.1% BSA. Wells were washed 3× with dIH₂O and were developed 20 minutes with PPMP solution ((10 gm phenolphthalein mono-phosphate (Sigma, St. Louis Mo.), 97.4 ml 2-amino-2-methyl-1-propanol (Sigma), 9.62 ml dIH₂O, 21 ml HCl)). Color development was stopped with 50 μl 0.2 M Na₂HPO₄ and the OD₅₅₀ was read on a Bio-Tek Instruments PowerWave 340 Microplate Spectrophotometer (Winooski, VT). Nominal antibody units were assigned to the standard pool and the concentrations of anti-domain 1 antibody (units/ml) in test sera were derived from the standard curve. Percent suppression of anti-domain 1 antibody by Multivalent platform conjugate, using Conjugates 200, 201, 202 and 203 treatment was calculated by comparison to PBS-treated controls. The Results are shown in Table 1, below.

TABLE 1
Percent Suppression of Anti-Domain 1 Antibody in Immunized Rats

| | nanomoles drug/rat | | |
|---|---|---|---|
| Compound | 0.17 | 1.7 | 17 |
| 200 | 61 | 82 | 89 |
| 201 | 34 | 73 | 86 |
| 202 | 72 | 89 | 96 |
| 203 | 73 | 93 | 94 |

By definition PBS control = 0% suppression

The half life of compounds in in rat plasma was also determined. Compounds were radiolabeled with ¹²⁵I using the iodine monochloride method. Contreras et al., 1983, Methods in Enzymology 92: 277-292. Labeled compound was injected i.v. and plasma samples were collected periodically over 24 h. The amount of drug in plasma was detected using a Packard Instruments Model Cobra gamma counter (Downers Grove, Ill.). Pharmacokinetic parameters were calculated using WinNonLin software (Pharsight Corp., Mountain View, Calif.) and the plasma half-life was determined using the formula t_{1/2}=0.693(MRT). The results are shown below in Table 2.

TABLE 2
Compound half life in rat plasma (hours)

| | |
|---|---|
| 204 | 8 |
| 200 | 20.2 |
| 201 | 9.8 |
| 205 | 14 |
| 202 | 18.4 |
| 203 | 20 |

1-53. (cancelled)

54. A valency platform molecule having a formula selected from the group consisting of:
R^{c}[O—C(═O)—NR¹-G₂—(ONH₂)ₙ]_{y};
R^{c}[C(═O)—NR¹-G₂—(ONH₂)ₙ]_{y};
R^{c}[NR¹—C(═O)-G₂-(ONH₂)ₙ]_{y};
R^{c}[NR¹—C(═O)—O-G₂—(ONH₂)ₙ]_{y};
R^{c}[R¹C═N—O-G₂—(ONH₂)ₙ]_{y};
and,
R^{c}[S-G₂(ONH₂)ₙ]_{y};
wherein:
y is 1 to 16;
n is 1 to 32;
R¹ is H, alkyl, heteroalkyl, aryl, heteroaryl or G₂—(ONH₂)ₙ;
R^{c} and each G₂ are independently organic moieties comprising atoms selected from the group consisting of H, C, N, O, P, Si and S atoms;
(ONH₂) of the formulas above is a terminal aminooxy group; and
wherein the valency platform molecule comprises at least four of said terminal aminooxy groups.

55. The valency platform molecule of claim 54, wherein R^{c} and each G₂ are independently selected from the group consisting of:
hydrocarbyl groups consisting only of H and C atoms and having 1 to 5,000 carbon atoms;
organic groups consisting only of carbon, oxygen, and hydrogen atoms, and having 1 to 5,000 carbon atoms;
organic groups consisting only of carbon, oxygen, nitrogen, and hydrogen atoms, and having from 1 to 5,000 carbon atoms;
organic groups consisting only of carbon, oxygen, sulfur, and hydrogen atoms, and having from 1 to 5,000 carbon atoms; and
organic groups consisting only of carbon, oxygen, sulfur, nitrogen and hydrogen atoms and having from 1 to 5,000 carbon atoms.

56. The valency platform molecule of claim 55, wherein R^{C} and each G₂ are independently selected from the group consisting of:
hydrocarbyl groups consisting only of H and C atoms and having 1 to 500 carbon atoms;
organic groups consisting only of carbon, oxygen, and hydrogen atoms, and having 1 to 500 carbon atoms;
organic groups consisting only of carbon, oxygen, nitrogen, and hydrogen atoms, and having from 1 to 500 carbon atoms;
organic groups consisting only of carbon, oxygen, sulfur, and hydrogen atoms, and having from 1 to 500 carbon atoms; and
organic groups consisting only of carbon, oxygen, sulfur, nitrogen and hydrogen atoms and having from 1 to 500 carbon atoms.

57. The valency platform molecule of claim 54, wherein R^{c} is selected from the group consisting of a C₁₋₂₀₀ hydrocarbon moiety; a C₁₋₂₀₀ alkoxy moiety; and a C₁₋₂₀₀ hydrocarbon moiety comprising an aromatic group.

58. The valency platform molecule of claim 54, wherein R^{c} comprises an oxyalkylene moiety.

59. The valency platform molecule of claim 54, wherein R^{c} comprises an oxyethylene moiety.

60. The valency platform molecule of claim 54, wherein R^{c} comprises oxyethylene units:
—(CH₂CH₂O)ₙ—;
wherein n is 1 to 5,000.

61. The valency platform molecule of claim 54, wherein G₂ comprises a functional group selected from the group consisting of alkyl, heteroalkyl, aryl, and heteroaryl.

62. The valency platform molecule of claim 54, wherein G₂ comprises a functional group selected from the group consisting of a C₁₋₂₀₀ hydrocarbon moiety; a C₁₋₂₀₀ alkoxy moiety; and a C₁₋₂₀₀ hydrocarbon moiety comprising an aromatic group.

63. The valency platform molecule of claim 54, wherein G₂ comprises an oxyalkylene moiety.

64. The valency platform molecule of claim 54, wherein G₂ comprises an oxyethylene moiety.

65. The valency platform molecule of claim 54, wherein G₂ comprises oxyethylene units:
—(CH₂CH₂O)ₙ—;
wherein n is 1 to 500.

66. The valency platform molecule of claim 54, wherein each G₂ independently comprises a functional group selected from the group consisting of amine; amide; ester; ether; ketone; aldehyde; carbamate; thioether; piperazinyl; piperidinyl; alcohol; polyamine; polyether; hydrazide; hydrazine; carboxylic acid; anhydride; halo; sulfonyl; sulfonate; sulfone; imidate; cyanate; isocyanate; isothiocyanate; formate; carbodiimide; thiol; oxime; imine; aminooxy; and maleimide.

67. The valency platform molecule of claim 54 having the formula:
R^{c}[O—C(═O)—NR¹-G₂—(ONH₂)ₙ]_{y}.

68. The valency platform molecule of claim 54 having the formula:
R^{c}[C(═O)—NR¹-G₂—(ONH₂)ₙ]_{y}.

69. The valency platform molecule of claim 54 having the formula:
R^{c}[NR¹—C(═O)-G₂—(ONH₂)ₙ]_{y}.

70. The valency platform molecule of claim 54 having the formula:
R^{c}[NR¹—C(═O)—O-G₂—(ONH₂)ₙ]_{y}.

71. The valency platform molecule of claim 54 having the formula:
R^{c}[R¹C═N—O-G₂—(ONH₂)ₙ]_{y}.

72. The valency platform molecule of claim 54 having the formula:
R^{c}[S-G₂(ONH₂)ₙ]_{y}.

73. The valency platform molecule of claim 54, wherein each G₂—ONH₂ is independently selected from the group consisting of:

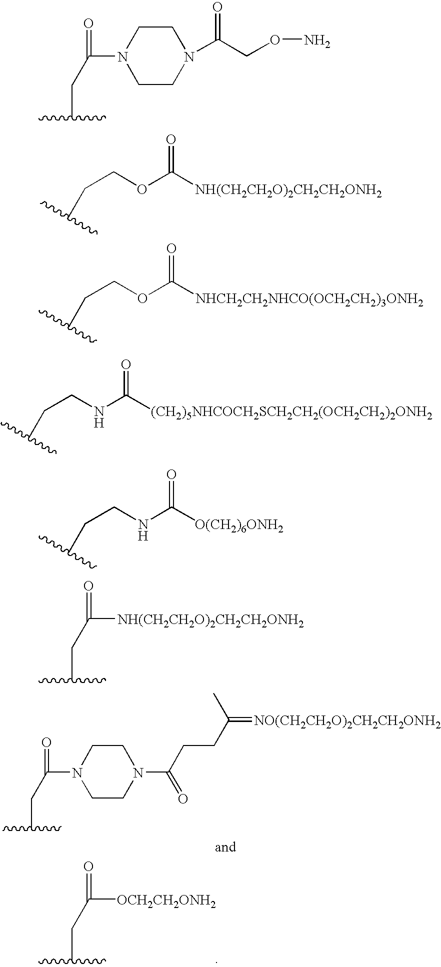

74. The valency platform molecule of claim 54 having a formula selected from the group consisting of: 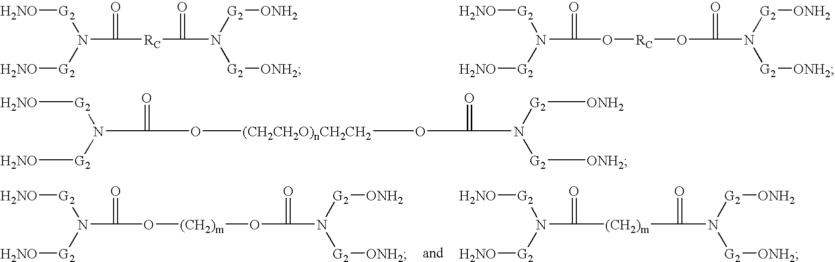
wherein n is 1 to 5,000 and m is 1 to 100.

75. The valency platform molecule of claim 74, wherein G₂ comprises an oxyethylene group.

76. A valency platform molecule of claim 54, having the structure: 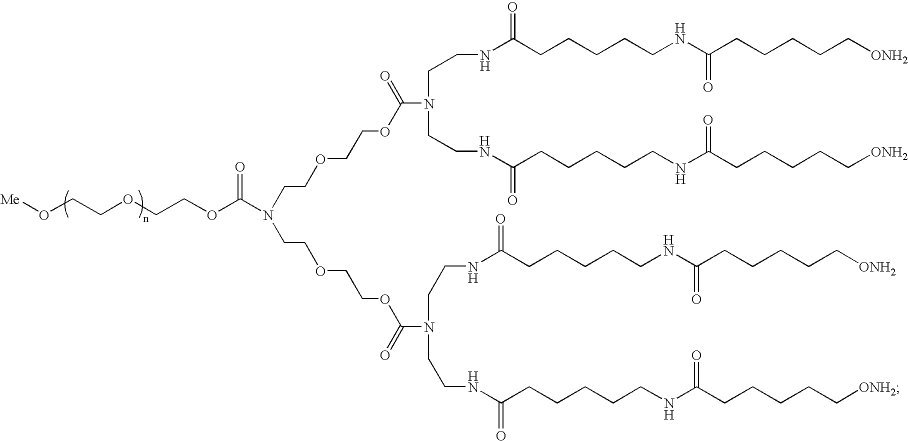
wherein n is about 503.

77. A valency platform molecule of claim 54, having the structure: 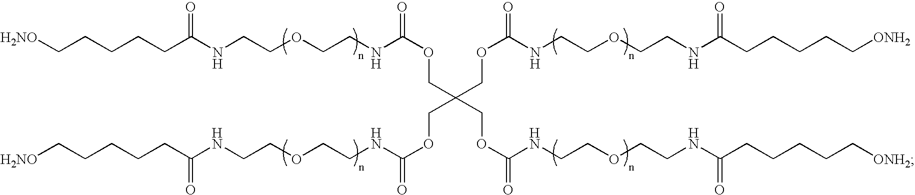
wherein n is about 112.

78. A valency platform molecule of claim 54 having the structure: 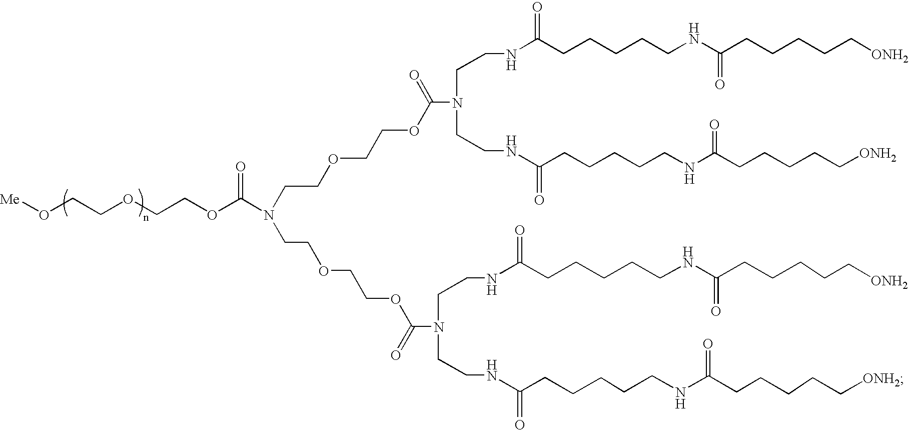
wherein the (CH₂CH₂O)ₙ moiety has a molecular weight of about 20 K g/mol.

79. The valency platform molecule of claim 67, wherein:
R^{c} is C(CH₂—)₄;
R¹ is H;
n is 1;
y is 4;
wherein G₂ comprises —(CH₂CH₂O)ₚ₋₁—CH₂CH₂—, wherein p is from 2 to about 500;
and wherein G₂ further comprises an amide moiety and a terminal aminooxy moiety.

80. The platform molecule of claim 67, wherein:
R^{c} is C(CH₂—)₄;
R¹ is H;
n is 1;
y is 4;
wherein G₂ comprises —(CH₂CH₂O)ₚ—, wherein p is from 200 to 500;
and wherein G₂ further comprises an amide moiety and a terminal aminooxy moiety.

81. The valency platform molecule of claim 54, having the following formula: 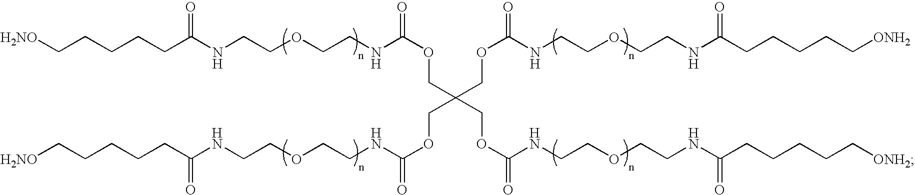
wherein each —(CH₂CH₂O)ₙ— moiety has a molecular weight of about 5 K g/mol.

82. The valency platform molecule of claim 60 or 74, wherein n is 1 to 500.

83. The valency platform molecule of claim 60 or 74, wherein n is 200 to 500.

84. The valency platform molecule of claim 54, wherein G₂ comprises an organic group consisting only of carbon, oxygen, and hydrogen atoms, and having from 1 to 5,000 carbon atoms.

85. The valency platform molecule of claim 84, wherein G₂ comprises oxyethylene units:
—(CH₂CH₂O)ₙ—;
wherein n is 1 to 200.

86. The valency platform molecule of claim 84, wherein G₂ comprises oxyethylene units:
—(CH₂CH₂O)ₙ—;
wherein n is 200 to 500.

87. The valency platform molecule of claim 54, wherein the valency platform molecule is symmetric.

88. The valency platform molecule of claim 54, wherein the valency platform molecule has a valence of four.

89. The valency platform molecule of any of claims 54, 60, 65, 67, 73 and 74, wherein the valency platform molecule further comprises one or more bivalent linker molecules that may be used for linking a biologically active molecule to the valency platform molecule, wherein the linker molecules comprise aminooxy groups that are optionally protected with an aminooxy protecting group, and wherein the bivalent linker molecules are bonded to the valency platform molecule such that a linkage bond is formed between the bivalent linker molecule and the valency platform molecule.

90. The valency platform molecule of claim 89, wherein the linkage bond that is formed is selected from the group consisting of: an amide linkage, a carbamate linkage, a thioether linkage and an oxime linkage.

91. The valency platform molecule of claim 90, wherein the linkage bond is formed by reacting the valency platform molecule with the bivalent linker molecule, wherein the bivalent linker molecule comprises a functional moiety that is selected from the group consisting of: amine, acid carbonate ester, thiol, aminooxy, and carboxylic acid.

92. The valency platform molecule of claim 54, wherein the valency platform molecule is dendritic.

93. The valency platform molecule of claim 67 or 68, wherein the valency platform molecule has a valence of four.

94. The valency platform molecule of claim 74, wherein n is 1 to 200.

95. A composition comprising two or more valency platform molecules according to claim 54, wherein the valency platform molecules have a polydispersity less than about 1.2.

96. A conjugate of a molecule according to any one of claims 54, 55, 60, 65, 67, 68, 73, 74, 79 or 80, and one or more biologically active molecules.

97. The conjugate of claim 96, wherein the biologically active molecules are selected from the group consisting of: oligonucleotides, peptides, polypeptides, proteins, antibodies, saccharides, polysaccharides, epitopes, mimotopes, enzymes, hormones, drugs, nucleic acids, lipids, fatty acids, and mixtures thereof.

98. The conjugate of claim 97, wherein the biologically active molecules comprise a polypeptide.

99. The conjugate of claim 97, wherein the biologically active molecules comprise a nucleic acid.

100. The conjugate of claim 97, wherein the biologically active molecules comprise an oligonucleotide.

101. The conjugate of claim 98, wherein the polypeptide lacks a T cell epitope.

102. The conjugate of claim 98, wherein the biologically active molecules comprise a domain 1 polypeptide of O₂GPI.

103. The conjugate of claim 102, wherein the polypeptide lacks a T cell epitope.

104. The conjugate of claim 102, wherein the conjugate comprises a linker that attaches the domain 1 polypeptide of O₂GPI to the valency platform molecule.

105. The conjugate according to claim 96, wherein the biologically active molecules interact specifically with proteinaceous receptors.

106. The conjugate according to claim 96, wherein the conjugate is a toleragen.

107. The conjugate according to claim 96, wherein the conjugate induces specific B cell anergy to an immunogen.

108. A method of making the conjugate according to claim 96, comprising: covalently bonding biologically active molecules to a valency platform molecule such that an oxime bond, or modified form thereof, is formed.

109. The method of claim 108, wherein the modified oxime bond is a reduced or alkylated oxime bond.

110. The method of claim 108, wherein the valency platform molecule comprises an aminooxy group and the biologically active molecules comprise a reactive functional group such that an oxime bond is formed upon bonding the biologically active molecules to the valency platform molecule.

111. The method of claim 110, wherein the reactive functional group is a carbonyl group of an aldehyde or ketone moiety.

112. The method of claim 111, wherein the biologically active molecules comprise a polypeptide; and, wherein the method comprises modifying the polypeptide prior to bonding with an aminooxy group on the valency platform molecule, such that the polypeptide comprises a terminal aldehyde group.

113. A pharmaceutical composition comprising the conjugate of any of claims 96, and 98-107, and a pharmaceutically acceptable carrier.
